(19) United States
(12) Patent Application Publication
(10) Pub. No.: US 2024/0082118 A1
(43) Pub. Date: Mar. 14, 2024

(54) TOPICAL COMPOSITIONS AND METHODS OF PREPARING THE SAME

(71) Applicant: LANDA LABS (2012) LTD., Rehovot (IL)

(72) Inventors: Sagi ABRAMOVICH, Ra'anana (IL); Gal AVIDOR, Rehovot (IL); Benzion LANDA, Nes Ziona (IL)

(73) Assignee: LANDA LABS (2012) LTD., Rehovot (IL)

(21) Appl. No.: 18/513,764

(22) Filed: Nov. 20, 2023

Related U.S. Application Data
(63) Continuation-in-part of application No. PCT/IB22/54627, filed on May 18, 2022.

(30) Foreign Application Priority Data
May 19, 2021 (GB) 2107130.3

(51) Int. Cl. A61K 8/06; A61K 8/37; A61K 8/46; A61K 8/55; A61K 8/67; A61K 8/85; A61K 9/10; A61K 9/51; A61K 31/355; A61K 31/765; A61K 47/14; A61K 47/20; A61K 47/30; A61Q 19/08

(52) U.S. Cl. CPC A61K 8/068; A61K 8/37; A61K 8/466; A61K 8/55; A61K 8/678; A61K 8/85; A61K 9/10; A61K 9/51; A61K 31/355; A61K 31/765; A61K 47/14; A61K 47/20; A61K 47/30; A61Q 19/08; A61K 2800/21; A61K 2800/413; A61K 2800/596

(57) ABSTRACT

There is disclosed a composition comprising a water-insoluble thermoplastic compound capable of stimulating collagen synthesis (CSSC), the CSSC having a molecular weight of more than 0.6 kDa and being dispersed in a polar carrier as nano-elements having an average diameter of 200 nm or less and a viscosity of 10⁷ mPa·s or less. The nano-elements of CSSC can be capable of stimulating the synthesis of skin proteins and/or enabling the delivery of active agents upon their application to a surface to be treated therewith. Methods for preparing the compositions and uses thereof are also provided.

### CROSS-RELATED APPLICATIONS

The present application is a Continuation-in-Part application of International Application No. PCT/IB2022/054627, filed on May 18, 2022, which claims Paris Convention priority from Great-Britain Patent Application GB 2107130.3, filed on May 19, 2021. The entire contents of the afore-mentioned applications are incorporated herein by reference for all purposes as if fully set forth herein.

### FIELD

The present disclosure relates to topical compositions for the treatment of surfaces, suitable in particular for dermatological purposes, including to achieve cosmetic and non-cosmetic effects. Methods of preparing these compositions are also disclosed.

### BACKGROUND

Skin plays an important role in protecting the body from hazards of the outer environment. It also displays the most visible signs of aging, such as drop in elasticity, tonicity and firmness leading to skin sagging, and such as superficial blemishes, or lesions, spanning from small lines to deep wrinkles.

The skin changes as a result of intrinsic and extrinsic factors. Intrinsic aging factors include genetics, cellular metabolism, hormones and metabolic processes. Such factors can cause diminished production of collagen and elastin, proteins widely present in the skin to ensure its structural integrity, and reduced production of glycosaminoglycans (GAGs), which are water-binding molecules contributing, together with elastin and collagen, to the skin matrix. Diminished functioning of the sweat and oil glands is another intrinsic process, which may also contribute to the skin becoming thinner and more fragile with age. Extrinsic factors include chronic light exposure, smoking, pollution, ionizing radiation, chemicals, toxins etc. They usually lead to thickening of the outermost skin layer (stratum corneum), pre-cancerous changes, likely leading to skin cancer, freckles and sun spots formation, as well as excessive loss of collagen, elastin, and GAGs. Together or alone, these processes give the skin the appearance of deep wrinkles, uneven tone, roughness and thin skin. Collagen, elastin, and GAGs, which can be referred to as structural skin polymers, do not only provide for the mechanical properties of the skin, but also fulfill biological functions, both in healthy and pathological conditions.

There are many approaches to reduce or delay skin aging, ranging from mild topical treatments to more extreme surgical ones. Early signs of aging can be treated with topical application of cosmetic products, including for example retinoids, vitamin C and α-hydroxy acids. Chemical peelings, dermabrasion, micro-needling, ultrasound energy devices, or laser resurfacing may be an option for moderate to severe skin damage. Deeper facial lines may be treated invasively, for instance by injecting botulinum toxin, dermal fillers, or skin polymers being depleted by the aging process, such as collagen itself, the size of these molecules preventing their delivery through the skin. Surgical interventions, such as a face lift, brow lift, or cosmetic surgery on the eyelids, are the more extreme measures taken against wrinkles and skin sagging.

Another approach used in anti-aging treatment of the skin involves increasing collagen synthesis in situ. There are many agents that are known to induce such synthesis, which can be administered orally, topically or parenterally. Orally-administered agents include food supplements such as vitamin C, ginseng, and nutrition-derived antioxidants (such as blueberries, cinnamon, certain herbs etc.), among others. Aloe vera and retinol, are two of the agents known to boost collagen synthesis while applied topically on the skin. Hydroxyapatite, on the other hand, may achieve such an effect only if administered by injection.

Topical compositions are considered more convenient for application, as well as safer, and typically more effective in covering large areas compared to compositions administered parenterally (involving pain and infection risks) or orally (where a first-pass metabolism should be overcome to retain efficacy). Hence, while injectable compositions having improved efficacy are still being sought, topical compositions are more desired, especially for anti-aging treatments, such as described above.

However, to be able to permeate the skin, cosmetically-active agents (i.e., cosmeceuticals) or medically-active agents (i.e., pharmaceuticals) in such topical compositions should be small enough, typically having a molecular weight (MW) of 500 g/mol or less, to be able to penetrate the skin barrier and achieve a satisfactory transdermal delivery. Such agents are preferably in the form of nano-materials, e.g., nano-fibers, nano-emulsions, nano-spheres, nano-capsules, nano-crystals, dendrimers, liposomes, nanotubes, etc., such as described in a review by Souto E. B. et al.; “Nanomaterials for Skin Delivery of Cosmeceuticals and Pharmaceuticals”; Applied Sciences, 2020, Vol. 10(5), 1594.

Besides the above exemplary compounds, known to boost synthesis of structural skin proteins, polymers (including proteins, and their fragmented/shorter versions known as peptides) have also been reported to promote the production of collagen, elastin, GAGs or other such molecules involved in maintaining the skin structural and functional integrity. Other polymers (or the same) may (alternatively or additionally) inhibit processes or enzymes (e.g., proteases) leading to the deterioration of natural skin proteins. For illustration, some peptides have been reported to boost collagen neo-synthesis, while others have been reported to inhibit collagenase, the enzyme responsible for collagen degradation.

Regardless of the type of biological activity, such materials (whether polymers or not) may have—positively stimulating neo-synthesis of structural skin proteins and/or negatively inhibiting down-regulators of such skin proteins—the end-result may range from reducing or delaying the diminution of skin proteins' amount, maintaining their level, or even increasing their presence. Such agents may be referred to herein as collagen-synthesis stimulating compounds (CSSC), or specifically as collagen-synthesis stimulating polymers (CSSP), the activity of such agents with respect to collagen (and/or other structural skin proteins) including not only the stimulation of its neo-synthesis but alternatively or additionally the prevention of its degradation.

Dermal fillers, also referred to as “volumizers”, temporarily “soften” wrinkles by filling the depressions underlying or surrounding wrinkles, hollows or creased lines on the surface of the skin. Dermal fillers may rejuvenate the skin by replacing the naturally disappearing structural skin polymers, restoring their level to an extent delaying the appearance of visible signs of aging. As such skin matrix components are polymers having relatively high molecular weights, their replacement generally requires injections, a method relatively expensive and triggering compliance issues. Taking for illustration hyaluronic acid (HA), it is normally present in the skin in the form of a polymer having a relatively high molecular weight of 500 kiloDalton (kDa) or more. Due to their size, these molecules are typically unable to traverse the skin barrier, so that cosmetic compositions aiming to deliver HA by transdermal application usually refer to a distinct class of polymers having a relatively lower molecular weight. The possible physiological roles of HA, or its relative potency, vary with the size of the polymer, larger ones being more potent for water retention, while smaller ones are considered more effective to boost neo-synthesis of structural skin polymers.

Thus, considering the molecular weight parameter out of the many factors that would additionally affect the potency of a product, there is, simply put, a conflict between compliance (increased for low molecular weight (LMW) HA) and efficacy (increased for high molecular weight (HMW) HA). HA is far from being the sole polymer of interest in the cosmetic realm to face a similar problem of ensuring convenient delivery, as achieved by topical application, while using molecules having a sufficiently high molecular weight to achieve sufficient or enhanced efficacy following transdermal penetration.

Various conventional methods for preparing nano-materials (e.g., polymeric), deemed suitable for skin penetration, are described in the literature. However, solvents typically remain residually trapped within the particles prepared by such methods. When the solvents being used are volatile organic compounds (VOCs), numerous drawbacks are known, since despite their relative volatility, such solvents cannot be fully eliminated beyond a residual level which can be non-negligible. First, the presence of a VOC within particles, even at residual level, may cause toxicity to the skin, regulatory guidelines accordingly requiring very limited amounts of residual VOCs, which might not be feasibly obtained by traditional methods. Secondly, as a VOC may gradually evaporate over time until it reaches its residual level, the particles' structure or properties may be altered during this period, consequently affecting their efficacy. While cumbersome methods have been developed in an attempt to address the residual entrapment of undesired solvents, the approaches reported so far fail to provide nano-particles, or even micro-particles having a narrow size distribution, and/or may lack commercial feasibility in view of their complexity, their failure to form stable dispersions, or their low encapsulation efficiency, when active ingredients are additionally desired.

As dermatological—pharmaceutical or cosmetic—products are constantly required in order to restore or maintain skin integrity (function and/or structure) for as long as possible protecting from environmental factors such as UV or toxic oxygen products, reducing dry skin conditions or fighting the cutaneous signs of ageing, there remains a need to provide dermatological compositions that resolve at least some of the problems described above. Advantageously, the novel compositions would permit a higher loading of CSSC, whether alone or in combination with additional ingredients having a beneficial dermatological activity, and/or the delivery of CSSC (in particular of CSSP) having a relatively high average molecular weight.

### SUMMARY

Aspects of the invention relate to dermatological compositions comprising a water-insoluble collagen-synthesis stimulating compound (CSSC), such as a collagen-synthesis stimulating polymer (CSSP), dispersed as nano-particles or nano-droplets in a polar carrier. Notably, the CSSC or CSSP molecules (which can be modified to enhance any of their desired properties) may have a molecular weight of 0.6 kDa or more. The compositions, which typically include at least one surfactant with the CSSC, the carrier, or both, may optionally further contain, in addition to the CSSCs or CSSPs, at least one active agent, such as specified herein-below.

While these dermatological compositions have been developed in order to overcome, inter alia, at least some of the drawbacks associated with present delivery of CSSC(s) (e.g., CSSP(s)) and/or particular active agent(s) to the skin, transdermal delivery by topical application being preferred, their suitability for parenteral delivery by sub-cutaneous injection in the area to be dermatologically treated (e.g., as dermal fillers) is not ruled out.

As used herein, the transdermal delivery of CSSC(s) and/or active agent(s) refers to the ability of such compounds to penetrate into the skin layers to target sites within the skin or just below it. In some embodiments, this route of administration of dermatological compositions according to the present teachings may further allow the delivery of such compounds across the skin for systemic distribution. Also disclosed are methods for preparing such topical or injectable dermatological compositions as well as uses thereof.

In a first aspect of the disclosure, there is provided a dermatological composition comprising nano-elements (i.e., nano-particles or nano-droplets) composed of at least one water-insoluble collagen-synthesis stimulating compound (CSSC) having a molecular weight of 0.6 kDa or more, the nano-elements being dispersed in a polar carrier, and having an average diameter (e.g., Dv50) of 200 nanometer (nm) or less.

In a second aspect of the disclosure, there is provided a dermatological composition comprising nano-elements composed of at least one water-insoluble CSSC, including (or plasticized by) a non-volatile liquid, the CSSC having an average molecular weight of 0.6 kDa or more, the nano-elements being dispersed in a polar carrier, and having an average diameter (e.g., Dv50) of 200 nm or less.

Nano-elements dispersed as described above are generally characterized by being discrete individual nano-elements, separated from one another, with minimal aggregation or agglomeration, thus maintaining their particle size as herein-described. The dispersibility can be confirmed by microscopic means, analyzing the particle size distribution of the nano-elements by standard methods (such as Dynamic Light Scattering (DLS)).

In some embodiments, each constituent of the nano-elements comprised in the present compositions (and/or obtained by the present methods) has a vapor pressure of 40 Pascal (Pa) or less, as measured at a temperature of about 20° C. As such, all materials forming the nano-elements have been set to be relatively non-volatile. In some embodiments, the vapor pressure of the nano-elements and of each of their constituents is 20 Pa or less, 5 Pa or less, or 1 Pa or less, as measured at a temperature of about 20° C.

In some embodiments, the nano-elements comprised in the present compositions (and/or obtained by the present methods) contain less than 2 wt. %, less than 1.5 wt. %, or less than 1 wt. % of a volatile organic compound (VOC), or a blend thereof, by weight of the nano-elements. In particular embodiments, the nano-elements contain less than 0.2 wt. %, less than 0.1 wt. %, less than 0.05 wt. %, or less than 0.02 wt. %.

In some embodiments, the bulk CSSC having certain characterizing properties, such as a viscosity and/or melting, softening and glass transition temperatures, when plasticized by, e.g., a non-volatile liquid (herein referred to as a “plasticized” or “swelled” CSSC) exhibits “second” characterizing properties, optionally being reduced as compared to the native ones. For instance, adding such a plasticizing agent to a bulk CSSC having a native viscosity of, e.g. 10⁷ millipascal-second (mPa·s), can result in the plasticized CSSC having a lower viscosity of 10³ mPa·s. The second characterizing property relates to the mixture of the CSSC with such agents inducing its plasticizing (including a non-volatile liquid, a surfactant, and even an active agent miscible therewith), referred to herein as a plasticized CSSC for brevity.

In some embodiments, the CSSC (and/or the plasticized CSSC) is characterized by at least one, at least two, or at least three of the following structural properties:
i. the CSSC and/or the plasticized CSSC is insoluble in the polar carrier;
ii. the CSSC and/or the plasticized CSSC is biodegradable and/or biocompatible;
iii. the CSSC and/or the plasticized CSSC has at least one of a melting temperature (Tm), a softening temperature (Ts), or a glass transition temperature (Tg) of at most 300° C., at most 290° C., at most 250° C., at most 200° C., at most 190° C., at most 180° C., at most 170° C., at most 150° C., or at most 120° C., said temperatures being either a first (i.e., native) Tm, Ts or Tg of the CSSC, or a second Tm, Ts or Tg of the CSSC if plasticized, or both;
iv. the CSSC and/or the plasticized CSSC has each respectively a first and/or second Tm of at least 0° C., at least 10° C., at least 20° C., at least 30° C., at least 40° C., at least 50° C., or at least 60° C.;
v. the CSSC and/or the plasticized CSSC has each respectively at least one of a first and/or second Tm between 0° C. and 300° C., between 10° C. and 300° C., between 20° C. and 300° C., between 0° C. and 290° C., between 20° C. and 250° C., between 20° C. and 200° C., between 30° C. and 190° C., between 30° C. and 180° C., between 40° C. and 180° C., between 40° C. and 150° C., between 50° C. and 150° C., or between 50° C. and 120° C.;
vi. The CSSC and/or the plasticized CSSC has each respectively a first and/or second Ts or Tg of −75° C. or more, −50° C. or more, −25° C. or more, 0° C. or more, 10° C. or more, 20° C. or more, 25° C. or more, 30° C. or more, 40° C. or more, 50° C. or more, or 60° C. or more;
vii. the CSSC and/or the plasticized CSSC has each respectively at least one of a first and/or second Ts or Tg between −75° C. and 300° C., between −75° C. and 290° C., between −50° C. and 250° C., between −25° C. and 200° C., between 0° C. and 290° C., between 0° C. and 180° C., between 10° C. and 250° C., between 20° C. and 300° C., between 20° C. and 250° C., between 20° C. and 200° C., between 30° C. and 190° C., between 30° C. and 180° C., between 40° C. and 180° C., between 40° C. and 150° C., between 50° C. and 150° C., or between 50° C. and 120° C.;
viii. the CSSC and/or the plasticized CSSC has each respectively a first and/or a second viscosity of 10⁷ mPa·s or less, 5×10⁶ mPa·s or less, 10⁶ mPa·s or less, 5×10⁵ mPa·s or less, 10⁵ mPa·s or less, 5×10⁴ mPa·s or less, 10⁴ mPa·s or less, 5×10³ mPa·s or less, or 10³ mPa·s or less, as measured at at least one temperature between 20° C. and 80° C., and at a shear rate of 10 sec⁻¹;
ix. the CSSC has a molecular weight of 0.7 kDa or more, 0.8 kDa or more, 0.9 kDa or more, 1 kDa or more, 1.5 kDa or more, 2 kDa or more, 2.5 kDa or more, 3 kDa or more, 3.5 kDa or more, 4 kDa or more, 4.5 kDa or more, 5 kDa or more, 5.5 kDa or more, 6 kDa or more, 6.5 kDa or more, or 7 kDa or more;
x. the CSSC has a molecular weight of 500 kDa or less, 300 kDa or less, 200 kDa or less, 100 kDa or less, 80 kDa or less, 50 kDa or less, 25 kDa or less, or 15 kDa or less; and
xi. the CSSC has a molecular weight between 0.6 kDa and 500 kDa, between 0.7 kDa and 300 kDa, between 0.8 kDa and 200 kDa, between 1 kDa and 100 kDa, between 2 kDa and 80 kDa, between 1.5 kDa and 500 kDa, between 2.5 kDa and 300 kDa, between 3 kDa and 200 kDa, between 3.5 kDa and 500 kDa, between 4 kDa and 500 kDa, between 5 kDa and 300 kDa, between 5.5 kDa and 300 kDa, between 6 kDa and 200 kDa, between 6.5 kDa and 200 kDa, or between 7 kDa and 200 kDa.

In some embodiments, the at least one structural property fulfilled by at least one of the CSSC and the plasticized CSSC is: property i) as above listed; property ii) as above listed; property iii) as above listed; property iv) as above listed; property v) as above listed; property vi) as above listed; property vii) as above listed; or property viii) as above listed; property ix) as above listed; property x) as above listed; or property xi) as above listed.

In some embodiments, the at least two structural properties fulfilled by at least one of the CSSC and the plasticized CSSC are: properties i) and ii); properties i) and v); properties i) and vii); properties i) and viii); properties i) and xi); properties ii) and v); properties ii) and vii); properties ii) and viii); properties ii) and xi); properties v) and viii), properties v) and xi); properties vii) and xi); or properties viii) and xi) of the above-listed properties.

In some embodiments, the at least three structural properties fulfilled by at least one of the CSSC and the plasticized CSSC are: properties i), ii) and iii); properties i), ii) and v); properties i), ii) and vii); properties i), ii) and viii); properties i), ii) and xi); properties i), iii) and vii); properties i), iii) and viii); properties i), iii) and xi); properties i), iv) and viii); properties i), iv) and xi); properties i), v) and vi); properties i), v) and vii); properties i), v) and viii), properties i), v) and xi); properties i), vi) and xi); properties i), vii) and ix); properties i), vii) and x); properties i), vii) and ix); properties i), vii) and xi); or properties i); viii) and xi) of the above-listed properties.

As used herein, a component is deemed to be “insoluble” within the liquid polar carrier (e.g., water) when its solubility within the carrier is less than 5 wt. %, and more typically, less than 4 wt. %, less than 3 wt. %, less than 2 wt. %, less than 1 wt. %, or less than 0.5%, by weight of the polar carrier, at a temperature of 20° C. Furthermore, such components are considered “miscible” within the CSSC by having a solubility within the CSSC of 5 wt. % or more (and more typically, 6 wt. % or more, 7 wt. % or more, 8 wt. % or more, 9 wt. % or more, or 10 wt. % or more) by weight of the CSSC they are mixed with.

In particular embodiments, the CSSC is a CSSP, the chemical compound being a polymer formed of repeating structural units, such monomers being either same (homopolymers) or different (random or block copolymers). In another particular embodiment, the polymer of the CSSP is a thermoplastic polymer. While non polymeric compounds typically have molecular weights of up to 2 kDa, generally not exceeding 1 kDa, CSSPs can be larger molecules of at least a few kDas.

As used herein, the term “nano-elements”, as used with respect to the structures containing inter alia the CSSC (plasticized or not), refers to relatively solid nano-particles or relatively liquid nano-droplets having an average diameter of 200 nm or less, 190 nm or less, 175 nm or less, 150 nm or less, 125 nm or less, 100 nm or less, 90 nm or less, 80 nm or less, 75 nm or less, 70 nm or less, or 50 nm or less, such structures being dispersed (e.g., as a result of nano-sizing) in a homogeneous medium, forming therein a nano-suspension. Such nano-elements have typically an average diameter of 2 nm or more, 5 nm or more, 10 nm or more, 15 nm or more, or 20 nm or more. In some embodiments, the average diameter of the nano-elements of the compositions according to the present teachings is between 2 nm and 200 nm, between 2 nm and 190 nm, between 2 nm and 175 nm, between 5 nm and 150 nm, between 5 nm and 125 nm, between 10 nm and 100 nm, between 10 nm and 90 nm, between 10 nm and 80 nm, between 15 nm and 75 nm, between 15 nm and 70 nm, or between 20 nm and 50 nm. The average diameter of the nano-elements can be determined by any suitable method and may refer to the hydrodynamic diameter of the elements as measured by DLS and established for 50% of the nano-elements by volume (Dv50).

In view of their intended use and/or method of preparation, CSSCs suitable for the present invention are advantageously relatively solid at room temperature (circa 20° C.) and up to body temperatures (e.g., circa 37° C. for human subjects). Such preferences are extended to the plasticized CSSC, which further takes into account the non-volatile liquid and its relative amount, or the presence of any other material affecting the thermal behavior of the product. As can be appreciated by persons skilled in the art, as the CSSCs can be thermoplastic polymers, a “relative solidity” of such materials, or such materials being “relatively solid”, at any particular temperature is referring to the fact that they are not necessarily solid but display a viscoelastic behavior. Without wishing to be bound by any particular theory, such feature of the CSSCs should ensure, to the extent necessary, that the nano-elements made therefrom are relatively non-sticky, facilitating their even distribution in a composition according to the present teachings. From the standpoint of skin penetration as required for transdermal delivery, it is believed that the relatively malleable/deformable nature of the CSSCs (whether intentionally plasticized or not to have a suitable viscosity as herein disclosed) facilitates the transfer of the nano-elements through the skin, the pathways across the stratum corneum, or down hair follicles or sweat glands being often narrow and tortuous.

In some embodiments, the first (native) viscosity of the CSSC is 10⁷ millipascal-second (mPa·s) or less, 10⁶ mPa·s or less, 10⁵ mPa·s or less, 10⁴ mPa·s or less, or 10³ mPa·s or less, as measured at at least one temperature between 20° C. and 80° C., and at a shear rate of 10 sec⁻¹.

In other embodiments, the first (native) viscosity of the CSSC, typically a CSSP, is higher than 10⁷ mPa·s under the aforesaid measuring conditions, being for instance of up to 10¹¹ mPa·s, in which cases the CSSC can be combined with a non-volatile liquid, for the purpose of plasticizing or swelling the CSSC so as to reduce its viscosity and facilitate its processing and incorporation as nano-elements into the present dermatological composition. Hence, in such embodiments, the composition further contains a non-volatile liquid in an amount suitable to at least lower the first (native) viscosity of the CSSC to a second (plasticized) viscosity of no more than 10⁷ mPa·s, as measured at at least one temperature between 20° C. and 80° C., and at a shear rate of 10 sec⁻¹.

In some embodiments, the CSSC having been plasticized by a non-volatile liquid (or any other compound having a plasticizing effect) exhibits a second viscosity being reduced as compared to the first viscosity, the second viscosity of the CSSC being of 10⁶ mPa·s or less, 10⁵ mPa·s or less, 10⁴ mPa·s or less, or 10³ mPa·s or less, as measured at at least one temperature between 20° C. and 80° C., and at a shear rate of 10 sec⁻¹.

The dermatological compositions of the present invention are in the form of a nano-suspension. Depending on the Tm or Ts of the CSSCs (either plasticized or having the desired viscosity in their native form), the composition can be at room temperature in the form of a nano-dispersion (i.e., if the Tm or Ts is above 20° C., e.g., between 25° C. and 80° C.), the nano-elements being relatively solid nano-particles, or in the form of a nano-emulsion (i.e., if the Tm or Ts is below 20° C.), the nano-elements being relatively liquid nano-droplets.

In some embodiments, the CSSC is plasticized and it exhibits (alone or in combination with any material added thereto as a mixture) at least one of a second Tm, Ts, or Tg, lower than the first respective Tm, Ts, or Tg of the unplasticized native CSSC. In some embodiments, the plasticized CSSC has a second Tm of at least 0° C. In other embodiments, the at least one of the second Ts or Tg of the plasticized CSSC is −75° C. or more. In other embodiments, the at least one of a second Tm, Ts, or Tg of the plasticized or swelled CSSC is at most 300° C. In some embodiments, the plasticized or swelled CSSC, and/or the mixture comprising it, has a second Tm being in a range from 0° C. to 290° C. In other embodiments, the second Ts or Tg of the plasticized CSSC and/or the mixture comprising it, is in a range from −75° C. to 290° C.

The CSSC (e.g., CSSP) can be a natural compound, even if artificially synthesized, or a synthetic compound, having no natural occurrence. Some compounds suitable for the present teachings may, under particular circumstances, assemble to form larger molecules considered then as polymers. Such compounds shall be referred to as polymerizable before such connections are made.

In some embodiments, the polymerizable CSSC is a natural compound selected from: resins, gums, gum-resins and combinations thereof. In a particular embodiment, the polymerizable CSSC is shellac or gum rosin.

In other embodiments, the CSSC is non-polymerizable, such as a quinone, in particular ubidecarenone, also called 1,4-benzoquinone or coenzyme Q10 (CoQ10).

In other embodiments, the CSSC is a CSSP, which may also be of synthetic or natural origin. In some embodiments, the thermoplastic polymer is a biodegradable polymer, selected from a group of polymer families comprising: aliphatic polyesters, polyhydroxy-alkanoates, poly(alkene dicarboxylates), polycarbonates, aliphatic-aromatic co-polyesters, isomers thereof, copolymers thereof and combinations thereof. While some of the afore-said polymers have natural counterparts, these exceptions are typically commercially available almost exclusively in artificially prepared form, so that the entire group is often considered as representative of synthetic polymers.

Alternatively, the thermoplastic polymer can be a non-biodegradable synthetic polymer such as a polyamide (PA), a polyethylene (PE), a poly(ethylene-co-acrylic acid) (PEAA), a poly(ethylene-co-methacrylic acid) (PEMAA), a poly(ethylene-co-n-butyl acrylate) (PEBA), a poly(ethylene-co-vinyl acetate) (PEVA), a polymethylmethacrylate (PMMA), a polypropylene (PP), a polysiloxane, a polystyrene (PS), a polytetrafluoroethylene (PTFE), polyurethane (PU), or a polyvinyl chloride (PVC).

In other embodiments, the CSSP is a natural biodegradable polymer selected from polysaccharides, lignin and combinations thereof.

In particular embodiments, the CSSC is selected from:
a) a polymerizable compound; and
b) a CSSP, having a molecular weight of 5 kDa or more.

In further particular embodiments, the CSSC is a biodegradable material such as shellac, gum rosin or a CSSP being an aliphatic polyester. In another further particular embodiment, the aliphatic polyester of the CSSP is selected from polycaprolactone, polylactic acid, poly(lactic-co-glycolic acid), poly(butylene succinate-co-adipate), isomers thereof, copolymers thereof and combinations thereof.

In yet a further particular embodiment, the CSSC is CoQ10.

In some embodiments, the non-volatile liquid that may be added to the CSSC to lower at least one of its first (native) viscosity, Tm, Tg and Ts is selected from a group comprising: monofunctional and polyfunctional aliphatic esters, fatty esters, cyclic organic esters, aromatic esters, fatty acids, terpenes, aromatic alcohols, aromatic ethers, aldehydes and combinations thereof. In some embodiments, the non-volatile liquid is an aliphatic or aromatic fatty ester. In particular embodiments, the non-volatile liquid is selected from: dibutyl adipate, C₁₂-C₁₅ alkyl benzoate, dicaprylyl carbonate, benzyl benzoate and dibutyl sebacate.

In other embodiments, when the polymer is a non-biodegradable synthetic polymer, the non-volatile liquid may additionally be selected from a group comprising mineral oils, natural oils, vegetal oils, essential oils, synthetic oils and combinations thereof, provided that they satisfy the present requirements. Non-limiting examples of synthetic oils include synthetic isoparaffins (e.g., Isopar™ M and Isopar™ V), C₁₂-C₁₅ alkyl ethylhexanoate, C₁₂-C₁₅ alkyl benzoate, or isononlyl isononanoate, to name but a few. Non-limiting examples of suitable vegetal oils include castor oil, corn oil, pomegranate seeds oil, or avocado oil, to name but a few. Non-limiting examples of essential oils include clove leaf oil, lavender oil, or oregano oil, to name but a few.

In some embodiments, the polar carrier in which the nano-elements comprising the CSSC can be dispersed includes water, glycols (e.g., propylene glycol, 1,3-butanediol, 1,4-butanediol, 2-ethyl-1,3-hexanediol and 2-methyl-2-propyl-1,3-propanediol), glycerol, precursors and derivatives thereof, collectively termed herein “glycerols”, (e.g., acrolein, dihydroxyacetone, glyceric acid, tartronic acid, epichlorohydrin, glycerol tertiary butyl ether, polyglycerol, glycerol ester and glycerol carbonate) and combinations thereof. In a particular embodiment, the polar carrier comprises water, consists of water or is water.

In some embodiments, the dermatological (e.g., topical) composition further comprises at least one surfactant, selected from an emulsifier and a hydrotrope. The surfactant(s) may be present in the nano-elements containing the CSSC (e.g., if being CSSC-miscible and polar-carrier-insoluble surfactant(s)), in the liquid phase containing the polar carrier (e.g., if being polar-carrier-soluble surfactant(s)), or in both (e.g., if being intermediate emulsifiers).

In some embodiments, the at least one surfactant is an emulsifier selected from a group comprising alkyl sulfates, sulfosuccinates, alkyl benzene sulfonates, acyl methyl taurates, acyl sarcocinates, isethionates, propyl peptide condensates, monoglyceride sulfates, ether sulfonates, ester carboxylates, fatty acid salts, quaternary ammonium compounds, betaines, alkylampho-propionates, alkyliminopropionates, alkylamphoacetates, fatty alcohols, ethoxylated fatty alcohols, poly (ethylene glycol) block copolymers; ethylene oxide (EO)/propylene oxide (PO) copolymers, alkylphenol ethoxylates, alkyl glucosides and polyglucosides, fatty alkanolamides, ethoxylated alkanolamides, ethoxylated fatty acids, sorbitan derivatives, alkyl carbohydrate esters, amine oxides, ceteareths, oleths, alkyl amines, fatty esters, polyoxylglycerides, natural oil derivatives and ester carboxylate.

In some embodiments, the at least one surfactant is a hydrotrope selected from a group comprising sodium dioctyl sulfosuccinate, urea, sodium tosylate, adenosine triphosphate, cumene sulfonate and salts (e.g., sodium, potassium, calcium, ammonium) of toluene sulfonic acid, xylene sulfonic acid and cumene sulfonic acid.

In some embodiments, the dermatological (e.g., topical) composition further comprises at least one skin-penetration enhancer. Such agents are typically found in the liquid phase in which the nano-elements containing the CSSC are dispersed.

In some embodiments, the dermatological (e.g., topical) composition further comprises at least one active agent within the nano-elements, the active agent being miscible with the CSSC and substantially insoluble in the polar carrier in which the nano-elements are dispersed (hence, referred to as a “carrier-insoluble active agent”). The carrier-insoluble active agent can be a blend of different agents, each having a same or different activity.

In some embodiments, the dermatological (e.g., topical) composition further comprises at least one active agent within the liquid phase including the polar carrier, the active agent being soluble in the polar carrier (hence, referred to as a “carrier-soluble active agent”).

As used herein, a material is deemed to be insoluble in a liquid carrier, being for instance a “polar-carrier-insoluble active agent” (or a “carrier-insoluble active agent”), if having a solubility within the carrier it is immersed in of less than 5 wt. % (and more typically, less than 4 wt. %, less than 3 wt. %, less than 2 wt. %, less than 1 wt. %, or less than 0.5%) by weight of the polar carrier, at a temperature of 20° C.

Conversely, a material is deemed to be soluble in a liquid carrier, being for instance a “polar-carrier-soluble active agent” (or a “carrier-soluble active agent”), if having a solubility within the carrier it is immersed in of 5 wt. % or more (and more typically, 6 wt. % or more, 7 wt. % or more, 8 wt. % or more, 9 wt. % or more, or 10 wt. % or more) by weight of the polar carrier, at a temperature of 20° C. As appreciated by a skilled person some materials having a sought activity can be polar-carrier-insoluble in one chemical form, and polar-carrier-soluble in another, salts of a material typically increasing its solubility.

In some embodiments, the dermatological (e.g., topical) compositions comprise more than one active agent in addition to the CSSC, the active agents being either in same or different phase. For illustration, a first active agent, being carrier-insoluble, can be contained within the nano-elements and a second active agent, being carrier-soluble, can be contained within the polar carrier phase.

The active agents to be included in the present compositions (whether carrier-insoluble or carrier-soluble) can have a cosmetic or a pharmaceutic purpose, having for illustration one or more activities selected from a group comprising: analgesic, anesthetic, anti-acne, anti-aging, anti-arthritic, anti-bacterial, anti-dandruff, anti-fungal, anti-inflammatory, anti-oxidant, anti-parasitic, anti-rheumatic, anti-viral, immunoregulatory, hair growth promoting or inhibiting, skin bleaching or tanning, and any like cosmetic or therapeutic activity beneficial to the skin or the treatment of its disorders, on the surface of the skin, within its layers or underneath it.

As used herein, the term “treatment” and its grammatical variants includes prevention, amelioration, attenuation, delay or arrest of progression and cure of the condition being treated; or may increase the efficacy of co-administered treatments directed to a same condition. For illustration, if a subject is to receive, in order to treat a skin wound, an autologous or heterologous skin graft, an heterograft or a synthetic skin substitute, the present compositions can improve the value of such treatment by promoting the neo-synthesis of skin proteins in the area being grafted and/or by locally providing immunosuppressors so as to facilitate the acceptance of the graft. The extent of treatment or co-treatment, as afore-exemplified, may be assessed, in some embodiments, by a diminution of the symptoms relevant to the condition being treated.

In some embodiments, the at least one carrier-insoluble active agent is selected from a group comprising: albendazole, benzoyl peroxide, bakuchiol, castor oil, ceramide, erythromycin, finasteride, funapide, ibuprofen, ketoconazole, lidocaine, macrolides, mebendazole, neem oil, retinol, salicylic acid, tadalafil, tetracyclines, tretinoin, vitamin A, vitamin D, vitamin E, vitamin K and plant extracts insoluble in the polar carrier, such active agents having inter alia anti-acne, anti-oxidant, anti-inflammatory, and/or anti-aging activity beneficial to the skin. In particular embodiments, the carrier-insoluble active agent that can be incorporated in the nano-elements of CSSC is retinol.

In some embodiments, the at least one carrier-soluble active agent is selected from a group comprising: azelaic acid, biotin, clindamycin, collagen, elastin, famciclovir, folacin, hyaluronic acid (HA), niacin, pantothenic acid riboflavin, thiamin, vitamin B12, vitamin B6, vitamin C and plant extracts soluble in the polar carrier, such active agents having anti-acne, anti-oxidant, anti-inflammatory, and/or anti-aging activity beneficial to the skin. In particular embodiments, the carrier-soluble active agent that can be dissolved in the polar carrier in which the nano-elements of CSSC are dispersed is HA.

Advantageously, the dermatological (e.g., topical) compositions of the present invention can have a relatively high concentration (e.g., 1 wt. % or more) of a CSSC (e.g., a CSSP) and/or of an optional active agent (e.g., retinol within the nano-elements or HA dissolved in the polar carrier), and/or the CSSC(s) and/or optional active agent(s) can have a relatively high molecular weight, as compared to conventional topical compositions comprising such ingredients. Without wishing to be bound by theory, the relatively higher loading of the CSSC(s) and/or optional active agent(s), and/or the relatively higher potency thereof (when MW-dependent), is expected to provide a higher gradient of concentration, favoring transdermal delivery, and ultimately cosmetic or pharmaceutic efficacy.

It is noted that nano-particles or nano-droplets of a CSSC having a particle size within the range of dimensions disclosed herein was found unexpectedly successful by the Inventors, as such forms of the CSSCs, in particular if being plasticized CSSPs, were expected to aggregate in view of their anticipated stickiness.

In a third aspect of the disclosure, there is provided a method for preparing a dermatological composition comprising a water-insoluble CSSC, the method comprising the steps of:
a) providing at least one water-insoluble CSSC, wherein the CSSC or each CSSC:
   i. has a molecular weight of at least 0.6 kDa;
   ii. has at least one of a first Tm, Ts, or Tg of 300° C. or less; and
   iii. has a first viscosity optionally higher than 10⁷ mPa·s, as measured at at least one temperature between 20° C. and 80° C., and at a shear rate of 10 sec⁻¹;
b) mixing the at least one CSSC with a non-volatile liquid miscible therewith, and optionally with at least one surfactant, the mixing being at a mixing temperature equal to or higher than the at least one first Tm, Ts, or Tg of the CSSC, whereby a homogeneous mixture including a plasticized CSSC is formed, the mixture having a second Tm, Ts, or Tg lower than the respective first Tm, Ts, or Tg, and a second viscosity lower than the first viscosity, the second viscosity being of 10⁷ mPa·s or less, as measured at at least one temperature between 20° C. and 80° C., and at a shear rate of 10 sec⁻¹;
c) combining the plasticized CSSC (optionally containing at least one surfactant) with a polar carrier; and
d) nano-sizing the combination of step c) by applying shear at a shearing temperature equal to or higher than at least one of the second Tm, Ts, or Tg of the plasticized CSSC, so as to obtain a nano-suspension, whereby nano-elements of plasticized CSSC are dispersed in the polar carrier;
the nano-elements having an average diameter (e.g., Dv50) of 200 nm or less.

In some embodiments of the third aspect, the second Tm, Ts, or Tg of the plasticized CSSC is lower than the respective first Tm, Ts, or Tg of the CSSC, by at least 5° C., at least 10° C., at least 15° C., at least 20° C., at least 25° C., at least 30° C., at least 35° C., at least 40° C., at least 45° C., or at least 50° C.

In some embodiments of the third aspect, the mixing temperature in step b) is higher than the at least one first Tm, Ts, or Tg of the CSSC by 5° C. or more, 10° C. or more, 20° C. or more, 30° C. or more, or 40° C. or more, as long as the mixing is performed at a temperature at which an insignificant part of the non-volatile liquid is boiled away. Assuming that the non-volatile liquid has a boiling temperature Tbₗ at the pressure of the mixing step, then in some embodiments the mixing temperature can additionally be lower than the boiling temperature Tbₗ of the non-volatile liquid. When the duration of mixing is sufficiently brief and/or the non-volatile liquid in sufficient excess, the mixing temperature can alternatively be at boiling temperature Tbₗ or above.

In a fourth aspect of the disclosure, there is provided a method for preparing a dermatological composition comprising a water-insoluble CSSC, the method comprising the steps of:
a) providing at least one water-insoluble CSSC, wherein the CSSC or each CSSC:
   i. has a molecular weight of at least 0.6 kDa;
   ii. has at least one of a first Tm, Ts, or Tg of 300° C. or less; and
   iii. has a first viscosity of 10⁷ mPa·s or less, as measured at at least one temperature between 20° C. and 80° C., and at a shear rate of 10 sec⁻¹;
b) combining the at least one CSSC with a polar carrier, and optionally with at least one surfactant; and
c) nano-sizing the combination of step b) by applying shear at a shearing temperature equal to or higher than the at least one first Tm, Ts, or Tg of the CSSC, so as to obtain a nano-suspension, whereby nano-elements of CSSC are dispersed in the polar carrier;
the nano-elements having an average diameter (e.g., Dv50) of 200 nm or less.

Advantageously, the afore-said methods do not require the solubilization of a water-insoluble CSSC within a solvent being a VOC, nor the addition of a VOC for any other reason, so that nano-elements obtained by the present methods may in some embodiments have a relatively low wt. % content of a VOC by weight of the nano-elements as detailed for the compositions.

In some embodiments of each of the third and fourth aspects, the CSSC provided in step a) is biodegradable and/or biocompatible.

In some embodiments of each of the third and fourth aspects, the shearing temperature is higher than the second Tm, Ts, or Tg of the mixture including the plasticized CSSC (or higher than the first Tm, Ts, or Tg, in case of an un-plasticized CSSC) by 5° C. or more, 10° C. or more, 20° C. or more, 30° C. or more, or 40° C. or more, as long as the nano-sizing is performed at a temperature or under any other conditions at which an insignificant part of the polar carrier is boiled away. Assuming that the polar carrier has a boiling temperature Tb_{c} at the pressure of the nano-sizing step, then in some embodiments, the shearing temperature can additionally be lower than the boiling point Tbₗ of the non-volatile liquid (if added) and/or lower than the boiling point Tb_{c} of the polar carrier, under the pressure at which the nano-sizing step is performed, by 5° C. or more, 10° C. or more, 20° C. or more, 30° C. or more, or 40° C. or more. When the duration of nano-sizing is sufficiently brief and/or the polar carrier in sufficient excess, the nano-sizing temperature can alternatively be at boiling temperature Tb_{c} or above.

In some embodiments of each of the third and fourth aspects, the obtained nano-suspension is a nano-emulsion, and the method comprises an additional step, wherein the obtained nano-emulsion is cooled to a temperature lower than at least one of the first or second Tm, Ts, or Tg of the CSSC or of the blend containing it. While such cooling may passively occur upon termination of nano-sizing, the temperature of the nano-suspension naturally decreasing over time to room temperature (or a lower temperature of storage, if desired), in some embodiments, the cooling is performed by actively lowering the temperature of the nano-emulsion by any suitable cooling method. Additionally, or alternatively, the cooling is performed under continued shearing or any other method maintaining the agitation of the composition. While the composition may remain a nano-emulsion following its active or passive cooling, in some embodiments, the composition can then be a nano-dispersion.

In some embodiments of the third and fourth aspects, a surfactant is added during step b) and/or during step c), the surfactant being either an emulsifier or a hydrotrope, as herein described.

In some embodiments of the third and fourth aspects, the polar carrier is not water, and the method further comprises a step of replacing at least part of the polar carrier by water.

In some embodiments of each of the third and fourth aspects, the method further comprises combining at least one CSSC-miscible/polar-carrier-insoluble active agent with the CSSC(s) (and the optional non-volatile liquid(s) and/or surfactant(s)) said combination being performed a) whilst mixing the CSSC with a non-volatile liquid and/or at least one surfactant, if applicable; b) by mixing the CSSC-miscible/polar-carrier-insoluble active agent with the plasticized CSSC (when applicable) prior to combining it with the polar carrier; or c) whilst mixing the CSSC (optionally plasticized) with the polar carrier or nano-sizing the compositions components, so as to obtain the nano-elements including the CSSC, the polar-carrier-insoluble active agent(s) being either in the nano-elements (if added as in a) or b)) or separately dispersed in the polar liquid phase (if added as in c)).

In some embodiments of each of the third and fourth aspects, the method further comprises dissolving at least one polar-carrier-soluble active agent within the polar carrier. The dissolution of such active agents can be performed at various steps during the preparation of the dermatological composition, depending on the resistance of the active agent to temperatures, mixing or shearing conditions applied at the envisioned step. Relatively resistant active agents can be added a) whilst combining the CSSC (or the plasticized CSSC) with the polar carrier; or b) whilst nano-sizing the compositions components so as to obtain the nano-elements including the CSSC. Alternatively, the active agent(s) which are soluble in the polar carrier, in particular if shear-sensitive, can be dissolved in the obtained nano-suspension, agents being relatively heat-sensitive being preferably dissolved in the polar carrier of the nano-emulsion or nano-dispersion after cooling.

In some embodiments of each of the third and fourth aspects, the method further comprises combining a first active agent, being miscible with the CSSC and insoluble in the polar carrier, with the CSSC, the combination being performed as aforesaid, and dissolving in the polar carrier a second active agent, being carrier-soluble, as aforesaid, the dermatological composition prepared thereby including a first active agent in the nano-elements containing the CSSC and a second active agent in the polar carrier phase of the nano-suspension in which the nano-elements are dispersed.

In some embodiments of each of the third and fourth aspects, the method further comprises adding a skin-penetration enhancer to the polar carrier phase of the nano-suspension. The addition of such a skin-penetration enhancer can be performed at various steps during the preparation of the dermatological composition, and typically as above described for the sake of incorporating an active agent soluble in the polar carrier.

In some embodiments of each of the third and fourth aspects, the CSSC, the polar carrier, and if desired for the preparation of the dermatological composition, the non-volatile liquid, the surfactant, the skin-penetration enhancer, the carrier-insoluble active agent and the carrier-soluble active agent, are substantially as described above and herein detailed.

It is noted in this context that while compounds have been for simplicity categorized according to their main role in the present invention, in particular with respect to the preparation methods, such functions are not exclusive one of the other. For illustration, a non-volatile liquid typically serving to plasticize the CSSC may also serve as a surfactant for the nano-elements, or as a solubilizing agent, increasing the miscibility of, e.g., a carrier-insoluble active agent within the CSSC; and a polar carrier (e.g., glycols) serving as liquid medium for the dispersed nano-elements or a surfactant (e.g., urea) intended to increase the dispersibility of the nano-elements, may additionally serve as a skin-penetration enhancer once the composition is applied on the skin. The predominance of one role over the other may depend upon a material inherent potency in the respective fields, but also on the relative presence of the material in the composition. For instance, a material deemed a carrier if constituting a significant enough part (e.g., more than 20 wt. %) of the liquid phase, may be considered to fulfil a distinct function if in a relatively lower amount, such amount being more adapted to its secondary roles.

In some embodiments, the dermatological compositions of the present invention can be prepared according to the methods herein disclosed and may further contain any additive conventionally present in such compositions.

While for the sake of illustration, the present compositions and methods are being described with respect to dermatological compositions intended for activities of relevance to the skin, such as being a product improving skin appearance and/or health, this should not be construed as limiting. The dermatological compositions according to the present teachings may additionally serve to transdermally deliver active agents targeting organs other than skin or disorders other than skin-specific, or they may serve to protect the skin from external factors. Such additional pharmaceutical agents can also be incorporated in the present compositions, either in the nano-elements if relatively carrier-insoluble, or in the polar carrier, if relatively carrier-soluble. Thus, the present dermatological compositions can be more broadly considered for the preparation of a veterinary product for the treatment of a non-human animal or a medicament for the treatment of a human subject.

Furthermore, the surfaces upon which the present compositions can be applied are not limited to the skin of a living subject. Considering that, e.g., in some embodiments the present composition can serve to deliver an active agent by releasing it from the nano-elements, the surfaces to be treated therewith can additionally be of inert objects or can be the environment or the surfaces (other than skin) of living subjects. For illustration of the former case, the composition can be applied to surfaces of buildings (or their inert contents, such as furniture or textiles), the compositions for example serving to release a pesticide over time. In such a case, the compositions can be considered industrial compositions.

Alternatively, the active agent to be released from the present compositions can be an agrochemical agent, in which instance the treatable surface can be the outer surface of a plant or a pest, or the soil or the water in which plants are growing or pests are present, and the composition can be considered an agrochemical composition.

All compositions, regardless of end-use, and all surfaces treatable therewith in accordance with the active agent present in the compositions are encompassed.

In a fifth aspect of the disclosure, there are provided uses for the present compositions for the preparation of a cosmetic product, an agrochemical product, an industrial product, a veterinary product, or a medicament for the treatment of a human subject, the composition being applied externally to a surface to be treated therewith, the composition comprising nano-elements dispersed in a polar carrier, according to the present teachings including the aspects and embodiments herein described.

In some embodiment, the product is a cosmetic product for achieving a cosmetic effect, the surface to be treated therewith typically being a skin of a mammalian subject (e.g., a human person) and the cosmetical composition being adapted for improving skin appearance (as inter alia resulting from stimulating the neo-synthesis of skin structural proteins and/or for preventing their degradation provided for by the CSSC). Such appearance improvement can include an anti-aging treatment, a skin protective treatment, a skin filling treatment, a skin smoothing treatment, and the like.

The cosmetic effects may additionally, or alternatively, be derived from the presence of a cosmetically active agent in the nano-elements.

While the present dermatological compositions may be injected where needed under the skin, their main use is for application onto the skin, allowing for the transdermal delivery of the CSSC (and of any other active agent present in the composition).

While for simplicity the effect of the present compositions, when deemed cosmetical, is referred to as “anti-aging”, for delaying, reducing, or preventing skin aging, this should not be construed as limiting, as processes similar to those leading to natural time-dependent skin aging are encountered in additional circumstances, such as degenerative disorders, or benign and malignant neoplasms, to name a few. Therefore, while for brevity the present invention is detailed for its cosmetic role and improvement of skin appearance (or postponement and/or diminution of deterioration of look), the compositions and the methods of preparation disclosed herein can have a broader beneficial impact, at least in the realm of dermatological treatment of conditions wherein one or more of the structural skin proteins that can be restored by the present compositions are pathologically reduced.

Thus, use of a dermatological composition comprising nano-elements of a water-insoluble CSSC dispersed in a polar carrier as herein-disclosed (optionally prepared by the methods of the present teachings) should be broadly understood as use of a cosmetical or a pharmaceutical composition achieving any desirable cosmetic or pharmaceutic improvement of the skin. Such effects, for which the present compositions can be beneficial, are generally manifested by improved skin appearance (use of the composition for, e.g., reducing the number of wrinkles and/or fine lines, improving skin elasticity, improving skin tonicity, combating wizened skin, combating flaccid skin, combating thinned skin, combating skin pigmentation, accelerating wound healing, promoting skin integrity, alleviating pain due to skin lesions, etc.) regardless of the cause of the phenomenon being treated by the composition.

In other embodiments, the dermatological compositions of the present invention may be used as pharmaceutical compositions for the improvement of skin health (and in turn often skin appearance as well), the compositions being considered as veterinary products when the surface to be treated therewith is a skin of a non-human animal or as a medicament when the treatment is applied to a human subject.

In alternative embodiments, the skin is not being treated per se but serves as substrate, the nano-elements including agents protecting the skin from external factors, whether living, physical or chemical. For illustration, the nano-elements may include insect repellents, UV-filters or nerve agents' protectors. Such agents may be considered pharmaceutic agents.

The present pharmaceutical compositions may also be used to treat organs other than the skin, while being delivered through the skin. In such a case, the nano-elements typically further contain an active agent adapted to delay, alleviate, prevent or treat a transdermally treatable disorder, wherein a pharmaceutical effect is achievable according to the active agents present in the compositions.

The skin upon which the cosmetic or pharmaceutical composition can be applied may be found in any relevant part of the body, such as on the face, scalp, trunk, or limbs.

In other embodiments, the product is an agrochemical and/or an industrial product, the surface to be treated therewith is the surface of an inert object, a plant or a pest, or the soil or the water in which plants are growing or pests are present, and the composition is an agrochemical composition or an industrial composition.

Additional objects, features and advantages of the disclosure will be set forth in the detailed description which follows, and in part will be readily apparent to those skilled in the art from the description or recognized by practicing the disclosure as described in the written description and claims hereof, as well as the appended drawings. Various features and sub-combinations of embodiments of the disclosure may be employed without reference to other features and sub-combinations.

### BRIEF DESCRIPTION OF THE DRAWINGS

Some embodiments of the disclosure will now be described further, by way of example, with reference to the accompanying figures, where like reference numerals or characters indicate corresponding or like components. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the disclosure. For the sake of clarity and convenience of presentation, some objects depicted in the figures are not necessarily shown to scale.

In the Figures:

FIG. 1 depicts a simplified schematic diagram of a method for preparing a dermatological composition according to an embodiment of the present teachings;

FIG. 2 shows the particle size distribution of nano-particles of PCL in a nano-dispersion prepared according to an embodiment of the present method, as measured by DLS and presented per volume;

FIG. 3 is a CryoTEM image of nano-particles of PCL in a nano-dispersion prepared according to an embodiment of the present method, the PSD of which was previously shown in FIG. 2;

FIG. 4 is a line chart showing the changes over time in facial wrinkles count in a group treated by a dermatological composition according to an embodiment of the present teachings compared to a group treated by a placebo composition presented in percentage of baseline values;

FIG. 5A is an image showing levels of collagen in the skin of a volunteer before applying a dermatological composition according to the present teachings (referred to as “Baseline”);

FIG. 5B is an image showing levels of collagen in the skin of the same volunteer as in FIG. 5A, as observed after one month of application of a dermatological composition according to the present teachings;

FIG. 6A is a picture of a volunteer's face, showing lines and wrinkles before applying a dermatological composition according to the present teachings (referred to as “Baseline”);

FIG. 6B is a schematic depiction of the lines and wrinkles shown in the picture of FIG. 6A;

FIG. 7A is a picture of the same volunteer's face as shown in FIG. 6A, after 3 months of application of a dermatological composition according to an embodiment of the present teachings, showing diminished lines and wrinkles;

FIG. 7B is a schematic depiction of the diminished lines and wrinkles shown in the picture of FIG. 7A;

FIG. 8A is a histological picture taken with a fluorescence microscope at a magnification of ×10 of a cross-section of pig ear skin treated with a dermatological composition according to the present teachings, the tested sample comprising nano-elements including a fluorescent marker;

FIG. 8B is a histological picture of a cross-section of pig ear skin treated with a control composition lacking a fluorescent marker, the picture being taken similarly as for FIG. 8A;

FIG. 9A is a picture of cortical neuronal cells following the application of a composition according to an embodiment of the present teachings, showing cellular penetration; and

FIG. 9B is a schematic depiction of the cellular penetration image of FIG. 9A.

### DETAILED DESCRIPTION

The present invention relates to dermatological (e.g., topical) compositions comprising nano-elements, e.g., nano-particles or nano-droplets, of a water-insoluble compound (in particular, a polymer) capable of stimulating neo-synthesis of structural skin proteins, such as collagen, elastin or glycosaminoglycans, and/or of inhibiting processes leading to their degradation, the nano-elements including the CSSC or CSSP being dispersed as nano-suspension in a polar carrier. Advantageously, the CSSC may have a molecular weight of 0.6 kDa or more, and more advantageously, a molecular weight of 3.5 kDa or more and can be, if desired, plasticized by or swelled with a non-volatile liquid, which can also be referred to as a plasticizing or swelling agent.

The nano-elements comprising the optionally plasticized CSSC may further include a surfactant and/or an active agent miscible therewith to respectively enable or increase the dispersibility of the nano-elements in the composition and/or to further enhance or modify the biological activity of the composition. Alternatively, or additionally, surfactant(s), active agent(s) and/or skin permeation enhancer(s) can be present in the polar carrier, if soluble therein.

When applied to the skin, the nano-elements comprised in the composition can remain on the skin surface or penetrate the skin barrier to a varying extent and provide, inter alia, an anti-wrinkle effect or any such effect restoring skin look, contributed at least in part thanks to the capacity of the CSSC in the nano-elements to maintain sufficient collagen presence in the skin. If an active agent added to the compositions is selected to be a cosmetic agent having a cosmetic effect similar to the CSSC, then the initial cosmetic effect that the sole CSSC might have provided can be enhanced (e.g., boosting an anti-wrinkle effect). However, a cosmetic agent may provide for a different cosmetic effect, the nano-elements combining the effect of the CSSC and of the cosmetic agent.

Methods for preparing such dermatological compositions and uses thereof (e.g., for cosmetical or pharmaceutical effects) are also disclosed.

Before explaining at least one embodiment in detail, it is to be understood that the disclosure is not necessarily limited in its application to the details of construction and the arrangement of the components and/or methods set forth herein. The disclosure is capable of other embodiments or of being practiced or carried out in various ways. The phraseology and terminology employed herein are for descriptive purpose and should not be regarded as limiting.

It is to be understood that both the foregoing general description and the following detailed description, including the materials, methods and examples, are merely exemplary of the disclosure, and are intended to provide an overview or framework to understanding the nature and character of the invention, and are not intended to be necessarily limiting.

### Biological Activity of CSSCs

The CSSCs that can be used in the present invention are selected for their ability to promote inter alia collagen formation within the skin and/or to prevent its degradation. Without wishing to be bound by any particular theory, it is believed that such compounds, upon their application and penetration into the skin, can trigger biological signals culminating in neo-synthesis of skin structural proteins. Such compounds may be biodegradable, being for instance biodegradable polymers, in which case the CSSCs can be broken down by certain biological mechanisms and cause local inflammation. This process may induce the formation of skin proteins such as collagen, for the purpose of healing the inflamed area, this newly synthesized collagen also contributing to the firmness of the skin. Alternatively, the CSSCs may be non-biodegradable, triggering a similar effect of skin protein (e.g., collagen) formation or protection, and consequent healing of the treated area, without having to undergo compound degradation.

In view of their intended use, the CSSCs are preferably biocompatible and typically biodegradable in a physiological environment, such as found following their application to the skin and their transdermal delivery. Suitable CSSCs can also be termed bioresorbable or bioabsorbable in the general literature, depending on their in vivo fate and prospective elimination from the body, but for simplicity all such compounds will be generally referred to herein as “biodegradable”. A CSSC is said to be biodegradable if breaking down relatively rapidly after fulfilling its purpose (e.g., by a bacterial decomposition process in the environment or by an in vivo enzymatic or metabolic process) to result in natural by-products. Biodegradable CSSCs are known, and new ones are being developed. Their relative biodegradability in various environments can be assessed by a number of methods, which depending on the conditions of interest can be procedures based on or modified from standards such as ASTM F1635.

Despite its propensity to naturally decompose under suitable physiological conditions, a biodegradable CSSC adapted to the present invention should be stable and durable enough for its intended use during storage and application, which can be particularly challenging if this use involves conditions that would enhance biodegradability. For example, spreading topical compositions containing CSSCs as a thin layer on the skin is expected to form a high surface area, which might increase the exposure of the resulting film to factors (e.g., light, chemicals, or micro-organisms) promoting the degradation of the CSSC before it is able to penetrate the skin and reach its target, hence, the selection of suitable CSSCs for the dermatological (e.g., topical) compositions of the present invention should take these factors into consideration.

The CSSCs used in the compositions are also biocompatible, and as such, they operate without eliciting undesirable local or systemic effects in the recipient of the compositions comprising them. Standard methods for assessing biocompatibility are known, such as the ones specified in ASTM F748 and ISO 10993.

As the compounds encompassed by the term CSSCs may additionally serve to deliver or release on surfaces other than the skin active agents of relevance to non-dermatological (e.g., agrochemical or industrial) products, it is appreciated that no actual promotion of synthesis of skin structural proteins are expected in such context. Then the term CSSC should be more generally understood to refer to a thermoplastic compound fulfilling the characteristics herein described in the context of CSSCs more specifically adapted to the skin.

### Insolubility

Besides their possible biodegradability, the CSSCs are preferably substantially non-soluble in the liquid phase of the composition including the polar carrier (e.g., water), in which they are dispersed as nano-elements.

As used herein, the solubility of a material (e.g., a CSSC, a non-volatile liquid, or an active agent) refers to the amount of such component that can be introduced into the liquid (e.g., polar) carrier, while maintaining the clarity of the liquid medium. The solubilities of specific components of the composition within any particular liquid are typically assessed in the sole polar carrier in absence of any other possible components of the compositions but may be alternatively determined with respect to the final composition of the liquid phase including the carrier.

CSSCs (or any other material of interest for the present invention) are deemed insoluble if their solubility in the polar carrier, or in the liquid phase containing it, is 5 wt. % or less, 4 wt. % or less, 3 wt. % or less, 2 wt. % or less, 1 wt. % or less, 0.5 wt. % or less, or 0.1 wt. % or less by weight of the carrier, or of the liquid phase. For illustration, no more than 5 g of a material that is non-soluble in a polar carrier would dissolve in 100 g of the carrier. This substantial insolubility, while typically measured at room temperature, should preferably apply at any temperature at which these ingredients are combined and processed, i.e., even at relatively elevated temperatures, the solubility of these compounds in the polar carrier should remain within the required ranges. A material satisfying these conditions can be referred to as a “polar-carrier-insoluble” material.

Such insolubility of the material is expected to prevent or reduce leaching out into their surrounding media of one or more of the CSSC (or of any other one of the constituents of the nano-elements of a CSSC mixture optionally plasticized or further including a surfactant and/or a carrier-insoluble active agent). Such leaching out, were the material soluble in the polar carrier, may affect the relative proportions of the constituents of the nano-elements, their size, or any other such parameter that may ultimately adversely affect the efficacy of the composition.

Regardless of the composition of the polar liquid phase including the polar carrier in which the CSSC is to be dispersed as nano-elements, the CSSC can first be characterized as being water-insoluble (i.e., having a solubility of less than 5 wt. % in water as typically established at room temperature).

### Molecular Weight

Advantageously, the present invention allows for the delivery of CSSCs having relatively high molecular weights as compared to compounds that may conventionally sufficiently penetrate the skin barrier to display any efficacy. CSSCs suitable for the present compositions, methods, and uses can have a molecular weight (MW) of 0.6 kDa or more, 0.7 kDa or more, 0.8 kDa or more, 0.9 kDa or more, 1 kDa or more, 1.5 kDa or more, CSSPs also displaying MW of 2 kDa or more, 2.5 kDa or more, 3 kDa or more, 3.5 kDa or more, 4 kDa or more, 4.5 kDa or more, 5 kDa or more, 5 kDa or more, 5.5 kDa or more, 6 kDa or more, 6.5 kDa or more, 7 kDa or more, or 10 kDa or more. Typically, their molecular weight does not exceed 2 kDa if the compound is not a polymer, though polymerizable compounds may be larger, CSSPs reaching MWs of up to 500 kDa, and being generally of 300 kDa or less, 200 kDa or less, 100 kDa or less, 80 kDa or less, 50 kDa or less, 25 kDa or less, or of 15 kDa or less. In another embodiment, the molecular weight of the CSSCs is between 0.6 kDa and 500 kDa, between 0.7 kDa and 300 kDa, between 0.8 kDa and 200 kDa, between 1 kDa and 100 kDa, or between 2 kDa and 80 kDa, between 1.5 kDa and 500 kDa, between 2.5 kDa and 300 kDa, between 3 kDa and 200 kDa, between 3.5 kDa and 500 kDa, between 4 kDa and 500 kDa, between 5 kDa and 300 kDa, between 5.5 kDa and 300 kDa, between 6 kDa and 200 kDa, between 6.5 kDa and 200 kDa, or between 7 kDa and 200 kDa.

As used herein, the term “molecular weight” (or “MW”) refers either to the actual molecular weight as can be calculated for a non-polymeric CSSC, which can also be expressed in grams/mole, or to the weight average MW of polymerizable CSSCs or CSSPs, which may be a blend of polymers each containing a slightly different number of repeating units, weight average MW of polymers being typically expressed in Daltons.

The molecular weight of the CSSCs can be provided by their suppliers and can be independently determined by standard methods including for instance gel permeation chromatography, high pressure liquid chromatography (HPLC), size-exclusion chromatography, light scattering or matrix-assisted laser desorption/ionization time-of-flight mass spectroscopy MALDI-TOF MS, some of these methods are described in ASTM D4001 or ISO 16014-3.

Such general rules concerning the molecular weight exemplified with respect to the CSSCs can be applied to any other constituent of the composition, whether polymeric or not, and in some embodiments, active agents added to the nano-elements may also be relatively large molecules having a MW of 0.6 kDa or more, and any other values as specifically recited for the CSSCs.

### Characterizing Temperatures

While the vast majority of non-polymeric compounds can be characterized by a melting temperature (Tm) at which they change from a solid phase to a liquid one, polymeric compounds can additionally or alternatively be defined by a glass transition temperature (Tg) if amorphous, pure amorphous polymers lacking a Tm. Pure crystalline polymers can be characterized by their Tm, semi-crystalline polymers often displaying two characterizing temperatures (e.g., Tg and Tm) reflecting the respective proportion of amorphous and crystalline parts in the molecule. Such polymers may also be defined by their softening temperature (Ts) midway the log step to melting. As the glass transition temperature describes the transition of a glass state into a rubbery state, and the softening temperature an intermediate inflection in the thermal analysis of a material, they typically relate to a range of temperatures or one at which the process will first be observed.

Therefore, depending on the chemical nature of the CSSC, the temperature that may characterize its thermal behavior can be at least one of a Tm, a Ts and a Tg. Hence, when a CSSC or the nano-elements comprising the same are defined as suitably having at least one of a first and/or second Tm, Ts and Tg within a particular range, the temperature considered is as relevant to the material. Some compounds may be identified by two such characterizing temperatures, in which case performing a method step at a temperature above any of the two temperatures could be above the lowest of the two (which would prolong the step) or the highest of the two (which would accelerate the step). Conversely, performing a method step at a temperature below any of the two temperatures could be below the highest of the two or the lowest of the two. Taking for illustration a semi-crystalline polymer that can be characterized by all three temperatures, Tm, Ts, and Tg in order of decreasing values, heating above Tg (i.e., above at least one), might be insufficient to reach Ts or Tm, while heating above Ts (i.e., above at least two), might be insufficient to reach Tm. Only heating above Tm would ensure that the temperature of heating is higher than all three temperatures that may characterize such exemplary polymer.

In some embodiments, the CSSCs suitable for the present compositions are characterized by at least one of a first (native) melting temperature (Tm), softening temperature (Ts) or glass transition temperature (Tg) being of at most 300° C., at most 250° C., at most 200° C., at most 180° C., at most 150° C., or at most 120° C.

In some embodiments, the first Tm of the CSSCs is at least 0° C., at least 10° C., at least 20° C., at least 30° C., at least 40° C., at least 50° C., or at least 60° C. In some embodiments, the first Tm of the CSSCs is between 0° C. and 300° C., between 10° C. and 300° C., between 20° C. and 300° C., between 20° C. and 250° C., between 20° C. and 200° C., between 30° C. and 190° C., between 30° C. and 180° C., between 40° C. and 180° C., between 40° C. and 150° C., between 50° C. and 150° C., or between 50° C. and 120° C.

In some embodiments, the CSSCs are characterized by at least one of a first Ts and a first Tg being of −75° C. or more, −50° C. or more, −25° C. or more, 0° C. or more, 10° C. or more, 20° C. or more, 25° C. or more, 30° C. or more, 40° C. or more, or 50° C. or more. In some embodiments, at least one of the first Ts and Tg of the CSSCs is between −75° C. and 300° C., between −50° C. and 250° C., between −25° C. and 200° C., between 0° C. and 180° C., between 20° C. and 300° C., between 20° C. and 250° C., between 20° C. and 200° C., between 30° C. and 180° C., between 40° C. and 180° C., between 40° C. and 150° C., between 50° C. and 150° C., or between 50° C. and 120° C.

Such thermal characteristics of a CSSC can be provided by its manufacturer or independently determined by standard methods, for instance, thermal analysis methods, e.g., Differential Scanning Calorimetry (DSC), such as described in ASTM 3418, ISO 3146, ASTM D1525, ISO 11357-3, or ASTM E1356.

Such measurements may be performed on the raw materials (e.g., bulk CSSC) or on intermediate (e.g., a mix) or end-products (e.g., the nano-elements) containing the CSSC. For this purpose, the nano-elements containing the CSSC may be isolated from the polar carrier and other agents present therein. The separation of the nano-elements from their liquid medium can be performed according to standards methods, such as by evaporation of the carrier by drying (e.g., in a vacuum oven) or freeze-drying, or by destabilization of the composition (e.g., by changing the pH) allowing the precipitation of the nano-elements. Nano-elements isolated by any suitable method from their samples can be additionally rinsed (e.g., with water) to remove residues that may affect the intended measurements, the nano-elements being then separated from the rinsing liquid. For instance, nano-elements precipitated out of their initial medium can be separated by centrifugation, the precipitate can be rinsed and centrifuged again in cycles until isolated and desirably rinsed nano-elements are obtained.

The characterizing temperatures (Tm, Ts or Tg) of a CSSC may be referred to as a “first” Tm, Ts or Tg, when relating to the native/unmodified compound, and may be referred to as a “second” Tm, Ts or Tg, when relating to the CSSC as modified. In some embodiments, the second Tm, Ts or Tg that can be measured on the blend obtained by mixing of the CSSC with a non-volatile liquid or any other desirable ingredient and/or on the nano-elements containing the same, is similar to the first respective Tm, Ts or Tg of the CSSC. Alternatively, the second Tm, Ts or Tg can be lower than the respective first characterizing temperature(s) of the CSSC, in which case the CSSC is considered to be plasticized or swelled.

### Polymeric and Non-Polymeric CSSCs

In some embodiments, the collagen-synthesis stimulating compounds (CSSCs) used in the present compositions, methods and uses are collagen-synthesis stimulating polymers (CSSPs). As the CSSPs are desirably adapted for biodegradation once delivered into the physiological environment of the skin (e.g., on, within or beneath it), such polymers generally contain hydrolysable or enzymatically cleavable sites. The presence of hydrolysable or otherwise cleavable sites is however non-essential, and polymers considered non-biodegradable may lack them.

In some embodiments, the CSSCs (or CSSPs) may be non-reactive and unable to build up more complex interactions, being only able to biodegrade, such as PCL. In other embodiments, the CSSCs (or CSSPs) may be biodegradable and have reactive moieties enabling interactions with additional different molecules, such as PLA, or additional same molecules, such as polymerizable natural resins having for instance aldehyde moieties.

Additionally, the CSSCs (or CSSPs) may be modified, e.g., by chemically binding functional groups, to provide for, enhance or modulate their properties.

Similarly, when the CSSCs are non-biodegradable thermoplastic compounds, such materials can also be reactive or not. Non-reactive non-biodegradable synthetic thermoplastic polymers can be for illustration polyethylene or polypropylene polymers, while polymers containing reactive groups can be e.g., ethylene-acrylic acid or ethylene-methacrylic acid copolymers.

Suitable CSSPs, which can be of natural or synthetic origin, are thermoplastic in nature, their shapes being capable of reversible modifications upon suitable heating and cooling.

Appropriate CSSPs can also be plasticized with a suitable non-volatile liquid, such optional treatment of the CSSPs facilitating their nano-sizing to an extent expediting transdermal delivery of the dispersed nano-elements.

Synthetic CSSPs can be biodegradable and selected from aliphatic polyesters, polyhydroxy-alkanoates, poly(alkene dicarboxylates), polycarbonates, aliphatic-aromatic co-polyesters, enantiomers thereof, copolymers thereof and combinations thereof.

To the extent that the monomers forming the CSSPs have chiral centers, all enantiomers and stereoisomers are encompassed. For illustration, lactic acid (2-hydroxypropionic acid, LA), exists as two enantiomers, L- and D-lactic acid, so that PLA has stereoisomers, such as poly(L-lactide) (PLLA), poly(D-lactide) (PDLA), and poly(DL-lactide) (PDLLA). A CSSP may therefore be a mixture of isomers of a same molecule or a specific stereoisomer (or a stereo copolymer).

In some embodiments, the CSSP is biodegradable and selected from a group comprising: aliphatic polyesters, such as poly-caprolactone (PCL), polylactic acid (PLA), poly(L-lactide) (PLLA), poly(D-lactide) (PDLA), poly(D,L-lactide) (PDLLA), polyglycolic acid (PGA), poly(lactic-co-glycolic acid) (PLGA), and poly(p-dioxanone) (PPDO); polyhydroxy-alkanoates (PHA), including polyhydroxybutyrate (PHB) (such as poly-3-hydroxy-butyrate (P3HB), poly-4-hydroxy-butyrate (P4HB), poly(3-hydroxybutyrate-co-3-hydroxyvalerate) (PHBV), poly-hydroxyvalerate (PHV), polyhydroxyhexanoate (PHH), and polyhydroxy-octanoate (PHO); poly(alkene dicarboxylates), such as poly(butylene succinate) (PBS), poly(butylene succinate-co-adipate) (PBSA) and poly(ethylene succinate) (PES); polycarbonates, such as poly-(trimethylene carbonate) (PTMC), poly(propylene carbonate) (PPC) and poly-[oligo(tetramethylene succinate)-co(tetramethylene carbonate)]; aliphatic-aromatic co-polyesters, such as poly(ethylene terephtalate) (PET) and poly(butylene adipate-co-terephtalate) (PBAT); their isomers, copolymers and combinations thereof.

In a particular embodiment, the biodegradable CSSP is or includes an aliphatic polyester, isomers, copolymers and combinations thereof. In a further particular embodiment, the CSSP is PCL. In another further particular embodiment, the CSSP is PLA. In a further particular embodiment, the CSSP is PLGA. In another further particular embodiment, the CSSP is PBSA.

Natural biodegradable CSSPs can be selected from polysaccharides (such as: cellulose, starch, chitin and chitosan), lignin and combinations thereof.

In some embodiments, the CSSP is non-biodegradable and selected from polyamide (PA), polyethylene (PE), poly(ethylene-co-acrylic acid) (PEAA), poly(ethylene-co-methacrylic acid) (PEMAA), poly(ethylene-co-n-butyl acrylate) (PEBA), poly(ethylene-co-vinyl acetate) (PEVA), polymethylmethacrylate (PMMA), polypropylene (PP), polysiloxane, polystyrene (PS), polytetrafluoroethylene (PTFE), polyurethane (PU), or polyvinyl chloride (PVC). In a particular embodiment, the non-biodegradable CSSP is selected from PA, PE, PEAA, PEMAA, PEBA, PEVA, substituted or modified versions thereof, ionomers thereof and combinations thereof.

Such above-mentioned polymers may be identified according to their respective characteristic functional groups as detectable by standard methods known to the skilled persons, for instance, by Fourier-transform infrared (FTIR) spectroscopy.

Non-polymeric CSSCs suitable for the compositions, methods and uses of the present invention can be natural or synthetic. Under suitable conditions, some natural CSSCs may react in a polymerization reaction, resulting in CSSPs.

Natural CSSCs that may be polymerizable can be selected from: natural resins (such as: shellac, rosin, damar, copal, sandarac, amber, mastic and manila); natural gums (either originating from gum-yielding trees, such as Acacia nilotica (babul), Acacia catechu (khair), Steruculia urens (kullu), Anogeissus latifolia (dhawra), Butea monosperma (palas), Bauhinia retusa (semal), Lannea coromandelica (lendia) and Azadirachta indica (neem), or originating from seeds of plants such as guar, tamarind and Cassia tora); natural gum-resins (such as asafoetida, myrrh, salai and guggul); and combinations thereof. In a particular embodiment, the natural polymerizable CSSC is shellac or gum rosin.

Non-polymerizable CSSCs include quinones. In a particular embodiment, the non-polymeric CSSC is coenzyme Q10 (CoQ10).

Additionally, a CSSC, can be a blend of different compounds, whether polymeric, polymerizable or not, the properties of the mixture (e.g., a characterizing temperature, a viscosity, etc.) satisfying the ranges set for a suitable individual compound. For instance, a CSSC or CSSP having a Tm, Ts or Tg out of a range previously deemed suitable (e.g., being lower than 20° C. or higher than 300° C.) may be combined with a CSSC or CSSP having a Tm, Ts or Tg adapted to “correct” the characterizing temperature of the obtained mixture to be suitable for the purpose of the present invention. For illustration, a CSSC can be a blend of polymers or a copolymer including at least one of the aforementioned CSSPs, such copolymers may contribute to the biocompatibility, biodegradability and mechanical and optical properties of the nano-elements.

The compositions of the present invention may contain nano-elements each prepared using various types of CSSCs. For instance, a composition may include nano-elements prepared using one type of CSSC combined with nano-elements prepared using a different type of CSSC. Alternatively, or additionally, the present compositions can contain nano-elements which differ in their function, for instance each type of nano-elements including a different optional active agent.

### Viscosity

CSSCs can alternatively (or additionally) be selected for their viscosity to be initially adapted to their shearing in the present methods for preparing the dermatological compositions. CSSCs suitable for the present methods, compositions and uses can typically have a viscosity which does not exceed of 10¹¹ millipascal-second (mPa s, being equivalent to a centipoise), and which is often of 5×10¹⁰ mPa s or less, 10¹⁰ mPa s or less, 5×10⁹ mPa s or less, 10⁹ mPa s or less, 5×10⁸ mPa s or less, 10⁸ mPa s or less, 5×10⁷ mPa s or less, 10⁷ mPa s or less, or of 5×10⁶ mPa s or less, as determined at at least one temperature between 20° C. and 80° C., and at a shear rate of 10 sec⁻¹.

It is to be noted that the temperature at which the viscosity of the CSSCs (or of the nano-elements comprising the same) can be measured to assess suitability may depend on the native characterizing temperatures of the CSSCs (i.e., first Tm, Ts or Tg) or on a second Tm, Ts or Tg characterizing the nano-elements. For instance, polycaprolactone, having a first Tm of about 60° C., may be plasticized to yield a second Tm of 50° C. or less, so that the viscosity of a plasticized PCL can be measured at about 50° C. A CSSC or nano-elements having a relatively higher first or second Tm may require the viscosity measurement to be performed at a temperature higher than 50° C. Conversely, if the CSSC or the nano-elements have a relatively lower first or second Tm their viscosity can be measured at a temperature lower than 50° C. Despite the wide ranges of characterizing temperatures of the present CSSCs (or nano-elements prepared therewith), it is believed that at at least one temperature within 30° C., within 20° C. or within 10° C. from 50° C. (i.e., between 20° C. and 80° C., between 30° C. and 70° C., or between 40° C. and 60° C.) the CSSCs or the nano-elements may display the disclosed dynamic viscosities.

When the viscosity relates to the native property of the isolated unmodified CSSC or a blend of CSSCs, it can be referred to as a “first viscosity”. When the viscosity relates to the CSSC as modified by its mixing with materials miscible therewith, it can be referred to as the “second viscosity” of the mixture containing the CSSC (or the nano-elements). For illustration, the second viscosity can be of a CSSP plasticized with a suitable non-volatile liquid. The viscosity of a material (whether modified or not by the presence of others) at any temperature of interest (or in a range thereof) can be determined by routine thermo-rheological analysis, such as described in ASTM D3835 or ASTM D440.

While a non-volatile liquid can be added to the CSSC or CSSP regardless of their native viscosity, such materials are typically used in the present compositions or methods when the CSSC has a relatively high first viscosity, such as higher than 10⁷ mPa·s, at at least one temperature between 20° C. and 80° C., and at a shear rate of 10 sec⁻¹. The non-volatile liquid (which may also be referred to as a plasticizing or a swelling liquid) is contained in the nano-elements including the plasticized or swelled CSSC, the liquid being typically adsorbed or otherwise retained by the CSSC.

Such “plasticizing” or “swelling” typically results in weight gain and/or a volume gain relative to the CSSC own mass or volume in its native form. Such plasticizing of the CSSC renders the plasticized CSSC softer and more malleable, as demonstrated by its reduced viscosity (i.e., the second viscosity being smaller than the first), facilitating their later nano-sizing to an extent expediting transdermal delivery of the resulting nano-elements.

Advantageously, the reduced viscosity should be adapted to the shearing process (e.g., shearing equipment, shearing temperature, etc.) being elected to nano-size the plasticized CSSC (e.g., a plasticized CSSP, mixed with any other materials miscible in the nano-elements). For instance, the non-volatile liquid and its proportion relative to the CSSC can be selected to lower the viscosity of the CSSC by at least half-a-log, or at least one log, and so on, as might be required. For illustration, if the CSSC has a first viscosity of 10⁸ mPa s, a plasticizing agent and its amount would enable a reduction of half-a-log if the CSSC so plasticized has a second viscosity of 5×10⁷ mPa s, or (if in a higher amount or if alternatively selected to be a more potent agent) would enable a reduction of one log if the CSSC so plasticized has a second viscosity of 10⁷ mPa s, as measured at at least one temperature between 20° C. and 80° C., and at a shear rate of 10 sec⁻¹.

In some embodiments, the first viscosity of the CSSC or the second viscosity of a plasticized CSSC is between 10² mPa·s and 10⁷ mPa·s, between 5×10² mPa·s and 10⁶ mPa·s, between 5×10² mPa·s and 10⁵ mPa·s, between 10³ mPa·s and 5×10⁴ mPa·s, or between 10³ mPa·s and 10⁴ mPa·s, as measured at at least one temperature between 20° C. and 80° C., and at a shear rate of 10 sec⁻¹. Viscosity can be measured with any suitable rheometer equipped with a spindle adapted to the intended range of viscosities at the appropriate shear rate.

Viscosity, however, is not only relevant to the method of preparation of the nano-elements. It is believed that nano-elements displaying the aforesaid viscosities (i.e., not exceeding 10⁷ mPa·s) are relatively malleable, such deformability being expected to expedite transdermal delivery of the nano-elements, when this is their intended use.

### Plasticization of CSSCs

While mentioned above for their effects on the viscosity of the CSSCs, to the extent reducing it would be desired, the non-volatile liquids that may be incorporated with the CSSC in the nano-elements may fulfil additional functions. Plasticizing or swelling, in particular of CSSPs, can be visually observed when the swelled polymer is at a temperature below melting. At higher temperatures, the effect of the non-volatile liquid can be detected via its plasticizing activity, which includes its ability to lower at least one of the temperatures characterizing the native CSSCs.

Decreasing a characterizing temperature of the CSSC, allows accordingly lowering processing temperatures at which the dermatological compositions can be prepared. For illustration, while the CSSC can have a first (native) Tm, Ts or Tg of 200° C. or less in absence of a suitable non-volatile liquid, the addition of such plasticizing agents may yield a plasticized CSSC having a second (modified) Tm, Ts or Tg, lower than the first, the second temperature being for instance of 95° C. or less. The drop in temperature afforded by the presence of the non-volatile liquid need not be as dramatic as illustrated, obviously depending on the value of the first Tm, Ts or Tg of the native CSSC, on the second Tm, Ts or Tg as may be desired to facilitate preparation of the composition and/or later penetration of the nano-elements, preferably on the boiling temperatures (Tb) of liquids which are to remain present in the composition (but not necessarily if the steps are brief enough and/or the liquids in excess in case some are boiled away), and/or on the concentration of the plasticizing agent with respect to the compound being plasticized.

Typically, the second Tm, Ts, or Tg of the plasticized CSSC (together with any other ingredients miscible with the CSSC) is lower than the respective first Tm, Ts, or Tg of the bulk CSSC, by at least 5° C., at least 10° C., at least 15° C., at least 20° C., at least 25° C., at least 30° C., at least 35° C., at least 40° C., at least 45° C., or at least 50° C.

A characterizing temperature of a CSSC may be satisfactorily reduced solely by non-volatile liquid. Alternatively, a CSSC-miscible active agent or surfactant, optionally additionally combined with the CSSCs, may further reduce at least one of their characterizing temperatures. Such agents possibly reducing the characterizing properties (temperatures and/or viscosity) of the CSSCs may be referred to as plasticizing agents.

If the mixture of CSSC(s) and plasticizing agent(s) further comprises ingredients (e.g., rheological modifiers, surfactants, preservatives, or any like material miscible with the CSSC which may have a plasticizing effect) that may impact the softening property of the resulting combination due to form, or be within, the nano-elements, then additionally and alternatively, the thermal characteristics deemed suitable for the present invention would apply to the entire mixture.

Hence, in some embodiments, the plasticized CSSC, or a mixture of components including it, has at least one of a second Tm, Ts and Tg of at most 300° C., at most 290° C., at most 250° C., at most 200° C., at most 190° C., at most 180° C., at most 170° C., at most 150° C., or at most 120° C.

In some embodiments, the nano-elements comprising the CSSC have a second Tm of 0° C. or more, 10° C. or more, 20° C. or more, 25° C. or more, 30° C. or more, 40° C. or more, 50° C. or more, or 60° C. or more. In some embodiments, the second Tm of the nano-elements comprising the CSSC is within a range of 0° C. to 290° C., 10° C. to 290° C., 20° C. to 290° C., 10° C. to 250° C., 20° C. to 250° C., 20° C. to 200° C., 30° C. to 190° C., 30° C. to 180° C., 40° C. to 180° C., 40° C. to 150° C., 50° C. to 170° C., 50° C. to 150° C., or 50° C. to 120° C.

In some embodiments, the nano-elements have at least one of a second Ts and second Tg being −75° C. or more, −50° C. or more, −25° C. of more, 0° C. or more, 10° C. or more, 20° C. or more, 25° C. of more, 30° C. or more, 40° C. or more, 50° C. or more, or 60° C. or more. In other embodiments, the at least one of the second Ts and Tg measured on the nano-elements comprising the CSSC is within a range of −75° C. to 290° C., −50° C. to 250° C., −25° C. and 200° C., 0° C. to 180° C., 0° C. to 290° C., 10° C. to 250° C., 20° C. to 250° C., 20° C. to 200° C., 30° C. to 190° C., 30° C. to 180° C., 40° C. to 180° C., 40° C. to 150° C., 50° C. to 170° C., 50° C. to 150° C., or 50° C. to 120° C.

Such thermal behavior and characterizing temperatures can be assessed while preparing the plasticized CSSC or mixtures including the same, or upon completion of the preparation method of the composition.

### Non-Volatile Plasticizing Liquids

While the role that the presence of non-volatile liquids may have in the efficacy of delivering the nano-elements including the CSSC is not ignored, the selection of such materials is mainly considered with a view of improving the processability of the CSSC, so as to facilitate the preparation and dispersion of the nano-elements within the polar carrier phase. Particularly suitable non-volatile liquids can both lower the viscosity of the CSSC and lower at least one of its Tm, Ts and Tg, as previously separately discussed. Advantageously, suitable non-volatile liquids improve the processability of the CSSC under conditions suitable for its shearing into nano-particles, the shearing temperature causing initially the formation of nano-droplets.

First, as implied by their names, agents adapted to plasticize a CSSC according to the present teachings are liquid at the temperature at which the CSSC is to be processed, namely at least at one of the temperatures of mixing with the CSSC and of shearing. Such liquid agents can also be liquid at room temperature.

To ensure that their effect would perdure, the plasticizing liquids are preferably non-volatile. As used herein, the term “non-volatile”, as can be used with regards to a liquid that may plasticize the CSSC, refers to liquids exhibiting a low vapor pressure, such as less than 40 Pascal (Pa, also Newton per square meter) at a temperature of about 20° C. In some embodiments, a non-volatile liquid (or any other ingredient for which a low volatility is preferred) can have a vapor pressure of 20 Pa or less, 5 Pa or less, 1 Pa or less, 0.1 Pa or less, or 0.01 Pa or less as measured at about 20° C. Such vapor pressure values are typically provided by the manufacturer of the liquid, but can be independently determined by standard methods, such as described in ASTM D2879, E1194, or E1782 according to the range of the vapor pressure. The low or substantially null volatility of the non-volatile liquids that may be used to plasticize a CSSC, if so desired, should preferably be maintained at the highest temperature at which the plasticized CSSC is processed. The use of such non-volatile liquids allows the CSSCs to remain in their plasticized or swelled state, without the risk of evaporation or elimination of the liquids, even at high temperatures of preparing the dermatological compositions according to the present methods.

Suitable non-volatile liquids are also characterized by having a boiling point (Tbi) that is higher than room temperature, higher than body temperature, and higher than an elevated temperature as may be desired for the preparation of the composition, as it is preferred that the liquids selected for plasticizing the CSSCs of the present invention do not substantially evaporate during or after the preparation of the dermatological compositions. That having been said, some boiling away may be tolerated if the mixing step at which the non-volatile liquids plasticize the CSSC is brief enough to ensure a residual presence as desired, and/or if the non-volatile liquids are added in sufficient excess to compensate for any partial boiling away that may take place.

For similar reasons of being desirably maintained with the CSSC to be plasticized therewith, and retained in the nano-elements comprising it, the non-polar liquid should preferably be unable to migrate to the polar carrier phase. Hence, suitable non-volatile liquids are essentially not miscible in such polar carriers (e.g., water), their solubility in the pure polar carrier or in the liquid phase containing it being as previously detailed for the CSSC, namely being of 5 wt. % or less, 4 wt. % or less, 3 wt. % or less, 2 wt. % or less, 1 wt. % or less, of 0.5 wt. % or less, or of 0.1 wt. % or less by weight of the carrier or the phase containing it.

Such non-volatile liquids need to be compatible with the CSSC of the composition (i.e., able to plasticize it: e.g., decreasing its Tm, Ts or Tg, and/or decreasing its viscosity). A non-volatile liquid adapted for a particular CSSC can be selected accordingly by routine experimentation. For instance, given a particular CSSC, various non-volatile liquids can be mixed with it, at one or more relative concentrations, and optionally at an elevated temperature, facilitating the plasticizing, and their effects on the CSSC being plasticized monitored by thermo-rheology (for their ability to decrease viscosity as a function of temperature) and by thermal analysis (e.g., by DSC, for their ability to decrease the Tm, Ts or Tg of the native CSSC). The non-volatile liquids most potent with respect to the particular CSSC can be selected accordingly.

Fundamentally, a material or a chemical composition is compatible with another if it does not prevent its activity or does not reduce it to an extent that would significantly affect the intended purpose. Such compatibility may be from a chemical standpoint, for instance, sharing similar functional chemical groups or each material having respective moieties that may desirably interact with one another. This kind of compatibility can be demonstrated by the combined materials forming a homogeneous mixture, rather than separate into different phases. A material would be incompatible with another if degrading it, and for illustration, a polar liquid phase would be incompatible with the nano-elements if dissolving them, destabilizing them, improperly charging them to have a charge incompatible with their intended use, and so on.

Materials should also be compatible with the methods used for the preparation of the composition, not being adversely affected by any of the steps the material would be subjected to in the process, nor being volatile (or otherwise eliminated) at the temperature(s) they are incorporated in the compositions. Understandingly, the materials need also be compatible with their intended use, which in the present case may include for illustration being biocompatible, non-irritating, non-immunogenic, and having any such characteristic providing for their regulatory approval at a concentration adapted for efficacious cosmetic or pharmaceutic compositions as herein-disclosed.

While compatibility is required between the CSSC and the non-volatile liquid, dissolution of the CSSC within the non-volatile liquid is unwanted, and as such, the non-volatile liquid does not function as a solvent with respect to the CSSC, but as a plasticizing agent intended to remain therewith.

It is believed that a solvent, generally being made of relatively small molecules, is able to enter between molecules of CSSC and distance them one from the other, to such an extent that the CSSC readily dissolves within the solvent, forming a homogeneous solution even at room temperature. As used herein, the term “solvent” refers to a liquid wherein more than about 5 wt. % of the CSSC can dissolve at room temperature.

A non-volatile (e.g., plasticizing) liquid, in contrast, being typically larger in size compared to molecules of solvents, would not readily separate CSSC molecules to form a solution at room temperature, at which it could at best provide for a preliminary swelling. Elevated temperatures are required to move the CSSC molecules sufficiently away from one another to allow enough non-volatile liquid to enter within the formed gaps. Even under such favorable conditions, mixing may be required to homogeneously form the plasticized CSSC. Such elevated temperatures can be equal to or higher than at least one of the first Tm, Ts, and Tg of the CSSC.

Non-volatile liquids suitable for the present invention can be selected from: monofunctional or polyfunctional aliphatic esters (such as dimethyl glutarate, dimethyl maleate, dimethyl methyl glutarate, dipropylene glycol dibenzoate and lactic acid isoamyl ester); fatty esters (such as 2-ethylhexyl lactate, benzyl benzoate, butyl butyryl lactate, C₁₂-C₁₅ alkyl benzoate, a mixture of caprylyl caprate and caprylyl caprylate, decyl oleate, dibutyl adipate, dicaprylyl carbonate, dibutyl maleate, dibutyl sebacate, diethyl succinate, ethyl oleate, glyceryl monooleate, glyceryl monocaprate, glyceryl tricaprylate, glyceryl trioctanoate, isopropyl myristate, isopropyl palmitate, L-menthyl lactate, lauryl lactate, n-pentyl benzoate, PEG-6 caprylic/capric glycerides, propylene glycol monolaurate, propylene glycol mono-caprylate, triacetin, triethyl citrate, triethyl o-acetylcitrate, tris(2-ethylhexyl) o-acetyl-citrate, tributyl o-acetylcitrate and tributyl citrate); cyclic organic esters (such as decanoic lactone, gamma decalactone, menthalactone and undecanoic lactone); aromatic esters (such as diethyl phthalate); fatty acids (such as caprylic acid, cyclohexane carboxylic acid, isostearic acid, lauric acid, linoleic acid, linolenic acid, myristic acid, oleic acid, palmitic acid and stearic acid); terpenes (such as citronellol, eugenol, farnesol, hinokitiol, linalool, menthol, menthone, neridol, terpineol and thymol); aromatic alcohols (such as benzyl alcohol); aromatic ethers (such as phenoxy ethanol); aldehydes (such as cinnamaldehyde); and combinations thereof.

In some embodiments, the non-volatile liquid that may be used to plasticize a CSSC as herein-disclosed is a polyfunctional aliphatic ester (PFAE), being a diester derivative of common dicarboxylic acids: namely adipic (C), azelaic (C₉) and sebacic (C₁₀) acids, the alcohol portion of the diesters generally falling in the C₃-C₂₀ carbon number range, including linear and branched, even and odd numbered alcohols. In particular embodiments, the non-volatile liquid, being a PFAE, is selected from dibutyl adipate (e.g., commercially available as Cetiol® B), C₁₂-C₁₅ alkyl benzoate (e.g., commercially available as Pelemol® 256), dicaprylyl carbonate (e.g., commercially available as Cetiol® CC) and dibutyl sebacate.

When the CSSC is non-biodegradable and/or when the surface to which the composition is to be applied is not skin, additional non-volatile liquids can be used. Such additional liquids can be mineral oils, natural oils, vegetal oils, essential oils, synthetic oils and combinations thereof, provided they preferably fulfill present requirements.

### Polar Medium

The liquid medium forming the continuous phase in which nano-elements including the CSSC are dispersed is polar. In some embodiments, the liquid phase consists essentially of a polar carrier, whereas in other cases additional components can be present within the polar carrier. Such additional components can be, for illustration, surfactants, carrier-soluble active agents, or skin permeation enhancers, as herein-detailed, or any other additives conventionally present in dermatological compositions. A polar carrier suitable for the present invention can be selected from a group comprising water, glycols (e.g., ethylene glycol, propylene glycol, dipropylene glycol, and 1,2-butanediol 1,3-butanediol, 1,4-butanediol, 2-ethyl-1,3-hexanediol and 2-methyl-2-propyl-1,3-propanediol), glycerols including glycerol, precursors and derivatives thereof (e.g., acrolein, dihydroxyacetone, glyceric acid, tartronic acid, epichlorohydrin, glycerol tertiary butyl ether, polyglycerol, glycerol ester and glycerol carbonate) and combinations thereof.

A polar medium may be formed of one or more suitable polar carriers, the resulting liquid being often referred to as an aqueous solution (or an aqueous phase) when water is the preponderant polar carrier. In some cases, a liquid deemed not sufficiently polar by itself (such as a fatty alcohol) can be present in the liquid phase in addition to the polar carrier(s), provided that the liquid insufficiently polar to form the entire liquid polar phase is a) soluble in the main polar carrier (e.g., having a water-solubility of 5 wt. % or more) so as to form a unique liquid phase therewith; and b) the overall polarity of the liquid phase is maintained. The polarity index of the resulting liquid phase may be of 3 or more, 4 or more, or 5 or more, water having for reference a polarity index of 9-10.

As the polarity index of a solvent refers to its relative ability to dissolve in test solutes, a liquid may additionally or alternatively be classified as polar or non-polar in view of its dielectric constant (εᵣ). Liquids having a dielectric constant of less than 15 are generally considered non-polar, while liquids having a higher dielectric constant are considered polar, the relative polarity of a liquid increasing with the value of the dielectric constant. Preferably, the polar carrier suitable for the present compositions has a dielectric constant of 20 or more, 30 or more, 40 or more, 50 or more, or 60 or more, as established at room temperature. For illustration, the dielectric constant of propylene glycol is 32, the dielectric constant of glycerol is 46, and the dielectric constant of water is 80. While for simplicity, this guidance is provided for a neat polar carrier, this in fact should preferably apply to the entire polar liquid phase prepared therefrom (e.g., including additional polar-soluble materials and/or consisting of a mixture of liquid carriers). Noticeably, a liquid polar phase can be constituted of a mix of formally polar solvents (e.g., having εᵣ≥15) with formally non-polar ones (e.g., having εᵣ<15), as long as their respective volume allows for the entire liquid phase to be polar (e.g., having εᵣ≥15). The dielectric constant of a liquid is typically provided by the manufacturer but can be independently determined by any suitable method, such as described in ASTM-D924.

As discussed, the composition of the polar liquid phase should be such that the nano-elements including the CSSC can remain essentially non-soluble and stably dispersed therein, with no significant leaching of the contents of the nano-elements into their surrounding medium, when undesired.

In order to reduce the elimination of the polar carrier during the preparation of the composition of the present invention (or avoid it altogether), polar carriers suitable for the present methods have a boiling temperature (Tbe) between 45° C. and 350° C., between 70° C. and 300° C., between 85° C. and 250° C., or between 90° C. and 250° C.

As the polar medium may comprise additional liquids and/or materials dissolved therein, the polar carrier can constitute at least 50 wt. %, at least 60 wt. %, at least 70 wt. %, at least 80 wt. %, or at least 90 wt. %, by weight of the liquid phase.

In a particular embodiment, the polar carrier comprises water (e.g., 45 wt. % water, 45 wt. % propylene glycol, and 10% of a fatty alcohol), consists of water (e.g., including between 51 wt. % and 80 wt. % of water), consists essentially of water (e.g., including between 81 wt. % and 99 wt. % of water), or is water.

### Surfactants

Some CSSCs may remain nano-dispersed in the dermatological composition in view of their inherent chemical properties, the nano-elements for instance having a charge sufficient to ensure repulsion of the particles, consequently ensuring their stable dispersion. Other CSSCs may alternatively or additionally remain nano-dispersed in view of being plasticized with a non-volatile-liquid additionally serving as a surfactant to a sufficient extent.

In such cases, and regardless of the underlying rationale, the nano-elements can be considered self-emulsifying (e.g., remaining as discrete individual nano-elements in the liquid medium in absence of a surfactant devoted to that purpose). The nano-elements are deemed dispersed, when the particles have reached a desirable size (and/or particle size distribution) and “stably dispersed” when the particles can retain a desirable size (or PSD) over time. The original size and PSD can be as determined upon completion of preparation of the nano-particles, or at any other desired timepoint. As size measurements may vary between repeats at a same time point, a size or PSD at a subsequent time point is considered substantially similar to values obtained at a previous time point, if being within 10% or within 5% of previously determined values. Therefore, the particles are considered stably dispersed if measurements of their sizes and/or size distribution do not vary by more than 10% or 5% for at least 1 day, for at least 2 days, or for at least 3 days. Such stability can be assessed under any desired storage conditions, either at room temperature, at cooler temperatures (e.g., 4-8° C.), or on the contrary at higher temperatures (e.g., 30-40° C.), when accelerated stability tests are desired.

While the present compositions can be stable as a result of the constituents of the nano-particles and/or their environmental conditions (e.g., pH of liquid medium), in some embodiments, the composition may further comprise at least one (dedicated) surfactant, for the nano-elements to remain dispersed (hence also in their intended size range).

Surfactants suitable for the purpose of the present invention lower the surface tension between the nano-elements containing the CSSCs and the environment in which they are immersed. Depending on their chemical formula (and on the CSSC and polar carrier being considered), the surfactants can be miscible with the CSSCs (but able to diffuse to its outer surface) or with the polar carrier wherein the nano-suspension is formed.

Surfactants suitable for the present compositions and methods are generally amphiphilic, containing a polar or hydrophilic part and a non-polar or hydrophobic part. Such surfactants may be characterized by Hydrophilic-Lipophilic Balance (HLB) values within the range of 1 to 35, wherein the HLB values, which generally imply compatibility with water systems, are typically provided on Griffin scale.

When the surfactants are relatively water-soluble, or for simplicity of preparation regardless of solubility, surfactants can be added to the liquid phase in which the nano-elements are present. When the surfactants are relatively water-insoluble, they can alternatively be incorporated together with the CSSCs of the nano-elements.

Suitable surfactants for the purpose of the present invention can be anionic, cationic, amphoteric or non-ionic surfactants.

Anionic surfactants can be selected from the group including: alkyl sulfates (e.g., sodium lauryl sulfate, ammonium lauryl sulfate and ammonium laureth sulfate); sulfosuccinates (e.g., disodium lauryl sulfosuccinate, disodium laureth sulfosuccinate, sodium dioctyl sulfosuccinate and their mixtures with sulfonic acids and lauramidopropyl betaine); alkyl benzene sulfonates (e.g., sodium tosylate, cumene sulfonate, toluene sulfonic acid, xylene sulfonic acid, cumene sulfonic acid and salts (e.g., sodium, potassium, calcium, ammonium) thereof); acyl methyl taurates (e.g., sodium methyl lauroyl taurate and sodium methyl cocoyl taurate); acyl sarcocinates (e.g., sodium lauroyl sarcosinate, sodium cocoyl sarcosinate and sodium myristoyl sarcosinate); isethionates (e.g., sodium butyl isethionate, sodium capryloyl isethionate and sodium lauroyl isethionate); propyl peptide condensates; monoglyceride sulfates; ether sulfonates and fatty acid salts (e.g., sodium stearoyl lactylate).

Cationic surfactants can be selected from the group including: quaternary ammonium compounds (e.g., benzalkonium chloride, stearalkonium chloride, centrimonium chloride, and trimethyl ammonium methyl sulfates).

Amphoteric surfactants can be selected from the group including: betaines (e.g., cocamidopropyl betaine); alkylamphopropionates (e.g., cocoamphopropionate); alkyliminopropionates (e.g., sodium lauraminopropionate); and alkylamphoacetates (e.g., cocoampho-carboxyglycinate.

Non-ionic surfactants can be selected from the group including: fatty alcohols (e.g., cetearyl alcohol); ethoxylated fatty alcohols (e.g., C₈-C₁₈ alcohol polyglycol, polyoxyl 6 stearate and polyoxyl 32 stearate); poly (ethylene glycol) block copolymers (e.g.; poloxamer); ethylene oxide (EO)/propylene oxide (PO) copolymers; alkylphenol ethoxylates (e.g., octylphenol polyglycol ether and nonylphenol polyglycol ether); alkyl glucosides and polyglucosides (e.g., lauryl glucoside); fatty alkanolamides (e.g., lauramide diethanolamine and cocamide diethanolamine); ethoxylated alkanolamides; ethoxylated fatty acids; sorbitan derivatives (e.g., polysorbates, sorbitan laurate, sorbitol, 1,4-sorbitan, iso-sorbide and 1,4-sorbitan triester, PEG-80); alkyl carbohydrate esters (e.g., saccharose fatty acid monoester); amine oxides; ceteareths; oleths; alkyl amines; fatty acid esters (e.g., ascorbyl palmitate, ethylene glycol stearate, polyglyceryl-6 esters, polyglyceryl-6 pentaoleate, polyglyceryl-10 pentaoleate and polyglyceryl-10 pentaisostearate); polyoxylglycerides (e.g., oleoyl polyoxyl-6 glycerides); natural oil derivatives; ester carboxylate (e.g., D-α-tocopherol polyethylene glycol succinate (vitamin E TPGS)); and urea.

These surfactants may be categorized into emulsifiers and hydrotropes, according to their mechanism of action. Emulsifiers readily form micelles (thus being characterized by a critical micelle concentration (CMC) value) and are believed to increase the dispersibility of the CSSC (or plasticized CSSC) when later combined with a polar carrier to yield a nano-suspension. As a rule, emulsifiers typically relate to surfactants ensuring the dispersion of one liquid into another, the liquids having opposite polarity, whereas dispersants relate to surfactants ensuring the dispersion of a solid into a liquid. As the present method may provide for nano-emulsions and nano-dispersions, surfactants referred to as emulsifiers at a step in which the nano-suspension is an emulsion, may in fact become dispersants, to the extent that an initial nano-emulsion later yields a nano-dispersion at a lower temperature. Hence, as used herein, the term “emulsifier(s)” also includes surfactants otherwise known as dispersants.

Emulsifiers that are lipophilic in nature, i.e., include a relatively large hydrophobic part, are more suitable to be combined with the CSSC (and any other material not miscible in the polar carrier, e.g., a non-volatile liquid), and may therefore be referred to as polar-carrier-insoluble emulsifiers (or surfactants, in general). Hence, such relatively hydrophobic emulsifiers are expected to be within the nano-elements of the composition. These relatively hydrophobic emulsifiers generally have HLB values of 9 or less, 8 or less, 7 or less, or 6 or less, on Griffin scale.

Emulsifiers that are more hydrophilic in nature have a relatively large hydrophilic part and would be more compatible with the polar phase of the composition, and may therefore be referred to as polar-carrier-soluble emulsifiers (or surfactants, in general). Such relatively hydrophilic emulsifiers generally have HLB values of 11 or more, 13 or more, 15 or more, 17 or more, or 20 or more.

Emulsifiers having HLB values within the range of 9 and 11 are considered “intermediate”, the hydrophobic and hydrophilic parts of such emulsifiers being fairly well-balanced. Such intermediate emulsifiers can be added in the present methods either to the CSSC or the polar carrier and may accordingly be found in the nano-elements or in their medium, the ability of a portion of such surfactants to migrate between the two phases being also envisioned.

Surfactant serving as emulsifiers are selected from a group comprising alkyl sulfates, sulfosuccinates, alkyl benzene sulfonates, acyl methyl taurates, acyl sarcocinates, isethionates, propyl peptide condensates, monoglyceride sulfates, ether sulfonates, ester carboxylates, fatty acid salts, quaternary ammonium compounds, betaines, alkylampho-propionates, alkyliminopropionates, alkylamphoacetates, fatty alcohols, ethoxylated fatty alcohols, poly (ethylene glycol) block copolymers; ethylene oxide (EO)/propylene oxide (PO) copolymers, alkylphenol ethoxylates, alkyl glucosides and polyglucosides, fatty alkanolamides, ethoxylated alkanolamides, ethoxylated fatty acids, sorbitan derivatives, alkyl carbohydrate esters, amine oxides, ceteareths, oleths, alkyl amines, fatty esters, polyoxyl-glycerides, natural oil derivatives and ester carboxylate.

In a particular embodiment, the emulsifier is selected from: vitamin E TPGS, poly (ethylene glycol) block copolymer, a mixture of polyoxyl 6 stearate type I, ethylene glycol stearates and polyoxyl 32 stearate type I (such as commercially available as Tefose® 63 from Gattefossé, France), mixtures comprising olive oil-derived extracts (such as commercially available under the brand Olivatis® from Medolla Iberia, Spain), ascorbyl palmitate, polyglyceryl-10 pentaoleate, polyglyceryl-10 pentaisostearate, oleoyl polyoxyl-6 glycerides (such as commercially available as Labrafil® M 1944 CS from Gattefossé, France), disodium laureth sulfosuccinate, disodium lauryl sulfosuccinate, a mixture of disodium lauryl sulfosuccinate, sodium C₁₄-C₁₆ olefin sulfonate and lauramidopropyl betaine (such as commercially available as Cola®Det EQ-154 from Colonial Chemical, USA) and a mixture of olive oil and glutamic acid (such as commercially available as Olivoil® glutamate from Kalichem, Italy).

While surfactants acting as emulsifiers are generally sufficient to stabilize nano-elements of the present compositions, the Inventors have found that when CSSCs are present at a relatively high concentration, as enabled by the invention, the addition of another type surfactants, namely hydrotropes, assisted in achieving a satisfactory stability.

Hydrotropes are also amphiphilic molecules, but contrary to emulsifiers, they contain a relatively shorter lipophilic chain. As the lipophilic portion of the hydrotropes is generally too short to allow micelle formation, the hydrotropes alternatively solubilize hydrophobic compounds in the polar carrier and permit co-emulsification, together with the emulsifier. Generally, hydrotropes are miscible mainly in the polar carrier phase (e.g., aqueous phase) of the nano-suspension, and are characterized by having HLB values of 10 or more, 12 or more, 15 or more or 18 or more.

Suitable hydrotropes can be selected from the group including: sodium dioctyl sulfosuccinate, urea, sodium tosylate, adenosine triphosphate, cumene sulfonate, toluene sulfonic acid, xylene sulfonic acid, cumene sulfonic acid and salts thereof.

In a particular embodiment, the hydrotrope is selected from: sodium dioctyl sulfosuccinate, urea and a salt of xylene sulfonic acid, such as ammonium xylenesulfonate.

### Active Agents

While the present dermatological compositions can be biologically active as a result of the presence of the CSSC by itself, their uses can be enhanced and/or modified by inclusion of active agents having similar functions, so as to enhance efficacy, and/or different functions, so as to broaden the scope of efficacy. In such a case, the nano-elements can be considered as nano-carriers for other active agents to be administered topically.

In some embodiments, the dermatological composition further comprises one or more polar-carrier-insoluble active agent(s). Such carrier-insoluble active agent(s) are generally comprised within the nano-elements, as they are miscible with the components included therein, i.e., CSSCs, and optional non-volatile liquid and emulsifier. The constituents of the nano-elements are miscible one with the other when forming a unique phase.

Such active agents which alone, and more so in combination with the CSSC, are expected to improve skin appearance and health are known and are only exemplified below. Information on additional compounds known to have activities favorable to cosmetic treatment of the skin can be found e.g., in the list of cosmetic ingredients that are generally recognized as safe (GRAS), maintained at the FDA's website, in the list provided in the European Cosmetic Ingredient Database (COSING), or in any similar authoritative list issued by relevant professionals in the cosmetic industry.

Active agents suitable for pharmaceutical treatment can be found e.g., in the FDA's Drugs@FDA database, in the European Medicines Agency (EMA)'s European Public Assessment Reports (EPARs) database, or in any similar authoritative list issued by relevant professionals in the pharmaceutic industry.

Active agents suitable for agrochemical or industrial compositions can be found e.g., in the Pesticides Product Information System (PPIS) maintained by the US Environmental Protection Agency (EPA), in the EU Pesticides Database maintained by the European Commission's Directorate-General for Health and Food Safety (DG SANTE), or in any similar authoritative list issued by relevant professionals in the agrochemical industry.

While selected according to the activity they are to provide to the compositions, the CSSC-miscible/polar-carrier-insoluble active agents may have secondary functions, and for illustration optionally act as plasticizers, when capable of decreasing the viscosity of the CSSC.

In some embodiments, and similar to the CSSC and non-volatile liquid described above, the optional CSSC-miscible/carrier-insoluble active agent should have a solubility of 5 wt. % or less, 4 wt. % or less, 3 wt. % or less, 2 wt. % or less, 1 wt. % or less, 0.5 wt. % or less, or 0.1 wt. % or less by weight of the polar carrier or the liquid phase including it.

The CSSC-miscible/carrier-insoluble active agent, incorporated into the nano-elements, may include, by way of non-limiting examples, albendazole, benzoyl peroxide, bakuchiol, castor oil, ceramide, erythromycin, finasteride, funapide, ibuprofen, ketoconazole, lidocaine, macrolides, mebendazole, neem oil, retinol, salicylic acid, tadalafil, tetracyclines, tretinoin, vitamin A, vitamin D, vitamin E and vitamin K. In particular embodiments, the carrier-insoluble active agents are bakuchiol, castor oil or retinol (e.g., for a cosmetic use), benzoyl peroxide, funapide or tadalafil (e.g., for a therapeutic use) and neem oil (e.g., as an insect repellent for humans).

In some embodiments, the dermatological composition further comprises one or more polar-carrier-soluble active agent(s), which would be dissolved within the polar carrier.

Exemplary active agents that are carrier-soluble and may be present in the polar liquid phase of the composition can be selected from: azelaic acid, biotin, clindamycin, collagen, elastin, famciclovir, folacin, hyaluronic acid (HA), niacin, pantothenic acid riboflavin, thiamin, vitamin B12, vitamin B6 and vitamin C. In particular embodiments, the carrier-soluble agent is cosmetically active HA, which is one type of glycosaminoglycan.

In some embodiments, both HA of low molecular weight (LMW), i.e., having a MW of less than 500 kDa, and HA of high molecular weight (HMW), i.e., having a MW of more than 500 kDa can be used. In some embodiments, the HA is a LMW HA having a MW of 400 kDa or less, 300 kDa or less, 200 kDa or less, or 100 kDa or less. In particular embodiments, the LMW HA has a molecular weight not exceeding 50 kDa, not exceeding 25 kDa, or not exceeding 10 kDa.

In some embodiments, the dermatological composition may comprise both a carrier-insoluble active agent and a carrier-soluble active agent.

Plant-based products and extracts, serving as active agents, may also be added to the compositions, and can either be carrier-insoluble or carrier-soluble. As used herein, the term “plant extracts” refers both to natural fractions isolated from any relevant part of any suitable plant (e.g., flowers, fruits, herbs, leaves, peels, roots, seeds, stems, etc.) and to the synthetic version of the active agents of the natural extracts. Plants, the natural products or extracts of which are traditionally used for cosmetic or therapeutic effects when applied to the skin, are known to the skilled persons and too numerous to be comprehensively listed. By way of example, a plant product or extract containing an active agent suitable for the present invention can be isolated or obtained from apple, bergamot, broccoli, cannabis, coffee, corn, curcumin, fennel, garden angelica, ginseng, grapefruit, honeybush, Japanese red pine, kale, orange, paprika, passion fruit, raspberry, rooibos, soybean, spinach, tea and tomatoes. Such plant extracts are known to have inter alia anti-acne, anti-oxidant, anti-inflammatory, and/or anti-aging activity. In a particular embodiment, the plant-based active agent used in the compositions is curcumin.

The carrier-insoluble and/or carrier-soluble active agents optionally added to the present dermatological compositions may have a cosmetic function, for instance, have a dermal filling effect, or may, by themselves, be capable of enhancing collagen synthesis (and/or reducing its degradation). Considering the collagen synthesis stimulating ability of the CSSCs or CSSPs within the nano-elements on their own, adding such active agents, which may serve a similar purpose, can result in a combined activity providing for an even higher collagen formation within the skin. Regardless of the exact cosmetic contribution of the active agents, the resulting dermatological compositions may be regarded as “cosmetically active”.

Alternatively, the active agents (carrier-soluble or -insoluble) may serve pharmaceutical purposes, rendering the compositions “pharmaceutically active”. All active agents, in particular if already used in existing topical formulations serving the therapeutic purposes exemplified herein, are encompassed. For illustration, the pharmaceutic agent capable of treating a skin condition (e.g., having an analgetic, anti-inflammatory and/or anti-arthritic activity) can be any known cortico-steroidal drug (e.g., amciconide, betamethasone, clobetasol, desonide, dexamethasone, fluticasone, hydrocortisone, methylprednisolone, mometasone, etc.) or any known non-steroidal drug (e.g., diclofenac, etodolac, flurbiprofen, ibuprofen, indomethacin, ketoprofen, ketorolac, meloxicam, nabumetone, naproxene, piroxicam, sulindac, etc.).

Hence, the nano-elements, in addition to their collagen-synthesis abilities, may be used as nano-carriers for cosmetic or pharmaceutical agents to be applied to the skin of a subject.

While active agents have been described above mainly for activities of relevance to the skin, this should not be construed as limiting the use of dermatological compositions according to the present teachings. Drugs that can be transdermally delivered by traditional means and targeting other organs or disorders can also be incorporated in the present compositions, either within the nano-elements if relatively water-insoluble, or dissolved in the polar carrier if relatively water-soluble. Such active agents, typically used in medicaments intended for human subjects, can serve various purposes, such as anti-depression, blood pressure regulation, contraception, and pain relief, to name a few.

Non-limiting examples of such active agents include amitriptyline, apomorhine, asenapine, buprenorphine, capsaicin, clonidine, deutetrabenazine, ensam, estrogen, fentanyl, gabapentin, granisetron, ketamine, nicotine, methimazole, methyl phenidate, nitroglycerine, oxybutynin, physostigmine, pramipexole, progestin, riluzole, rivastigmine, rotigotine, scopolamine, selegiline, sumatriptan, and testosterone.

Most of the active agents listed above to exemplify uses of relevance to the treatment of human subjects can also be used in veterinary products for the treatment of non-human animals. Dermatological compositions for veterinary uses can additionally contain different active agents adapted to the physiology of the animal to be treated, to the effect to be obtained (e.g., increasing meat, milk, or wool production), and/or to the animal specific disorder. For illustration, active agents more suited to veterinary products and uses include agents improving the productivity of the livestock or protecting the animals (e.g., various pesticides, such as methoxychlor and carbaryl; anti-infection agents, such as isoflupredone; and anti-bacterial agents, such as thiostrepton; to name a few).

Moreover, the present nano-elements can be used to deliver active agents to surfaces other than the skin of a living animal. In such cases, the active agents can be considered as agrochemicals and/or industrial agents that can be incorporated into the CSSC nano-elements, to be used in these fields. Nano-elements containing such agents can be used at least as acaricides, algicides, animal repellents, anthelmintics, anti-moth agents, avicides, bactericides, chemo-sterilants, fertilizers, fungicides, herbicides, insect pheromones, insect repellents, insecticides, molluscicides, nematicides, nitrification inhibitors, ovicides, plant growth activators, rodenticides, termicides and virucides.

While attempts have been made to classify the active agents according to the composition they may supplement or the particular activity they may contribute for use in cosmetic products, pharmaceutical products for veterinary or human use, agrochemical products or industrial products, it is stressed, as previously mentioned, that overlaps exist. For illustration, a fungicide may be used as a preservative in a cosmetic composition, as an active agent to treat a fungal infection on the skin of a living subject, as a transdermally deliverable active agent to treat a systemic fungal infection in animals, as an active agent to treat plant fungal infections or as an active agent to prevent the growth of molds on inert surfaces.

Regardless of their activity and intended use, active agents can be CSSC-miscible/carrier-insoluble (in which case they are incorporated within the nano-elements) or carrier-soluble (in which case, they can be dissolved within the polar carrier).

While selected according to the activity they are to provide to the present dermatological compositions, active agents may have secondary functions, and for illustration CSSC-miscible/polar-carrier-insoluble active agents may optionally act as plasticizers, when capable of decreasing the viscosity of the CSSC within the core of the nano-elements, or polar-carrier-soluble active agents may optionally act as surfactants, when capable of stabilizing the nano-elements in the liquid phase they are dispersed in.

### Skin-Penetration Enhancers

While the polar carriers may suffice to enable enough delivery of the CSSC nano-elements through the skin, and some surfactants (if present) can promote it, in some embodiments, the topical composition further comprises a skin-penetration enhancer.

Suitable skin-penetration enhancers can be selected from the group comprising: C₁-C₂₂ alcohols (such as short chains alcohols: ethanol, isopropyl alcohol, and hexanol, and fatty alcohols: octanol, decanol, lauryl alcohol, myristyl alcohol, oleyl alcohol and octyl dodecanol); amides such as 1-dodecylazacycloheptan-2-one (also known as laurocapram and commercialized as Azone®) and its analogues, N-alkyl-azacycloheptan-2-ones, where the alkyl has the general formula CₓH₂ₓ₊₁, X being an integer selected from 1, 3-10, and 14, azacycloheptan-2-ones N-substituted by branched and/or unsaturated chains, N-acylazepan-2-ones, substituted 2-(2-oxoazepan-1-yl) alkanoic acid and its esters, N-alkyl-azacycloheptan-2-thiones, N-alkyl-azacycloheptenones, 4-alkyl-1,4-oxazepan-5,7-dione; cyclic amines with alkyl having the general formula CₓH₂ₓ₊₁, X being an integer selected from 10-12, 14, 16, and 18; long-chain N-acylazepanes, dehydrogenated azacycloheptane derivatives, six-membered ring analogues such as: azacycloheptadienes, N-substituted piperidin-2-ones, derivatives of six-membered ring analogues of Azone®, esters of 2-(2-oxopiperidin-1-yl)acetic acid, N-substituted derivatives of 6-oxopiperidine-2-carboxylic acid, N-1-(2-alkylsulfanylethyl)-piperidine-3-carboxylic acids, (thio)morpholines, morpholine-dione derivatives, long-chain N-acyl-morpholines, long-chain N-morpholinylalkenones, five-membered ring analogues: long-chain N-acylmorpholines and morpholinoethanol derivatives, 1-piperazin-1-yl-alkan-1-ones and 1-(4-methylpiperazin-1-yl)-alkan-1-ones; aromatic esters (such as octyl salicylate and 2-ethylhexyl 4-(dimethylamino)benzoate); ether alcohols (such as 2-(2-ethoxy-ethoxy)ethanol); glycols; pyrrolidones (such as 2-pyrrolidone and N-methyl-2-pyrrolidone); and sulphoxides (such as dimethyl sulphoxide (DMSO) and decylmethyl sulphoxide).

Typically, skin-penetration enhancers, if present, are added to the polar liquid phase. To the extent that they would be alternatively provided within the nano-elements, attention should be given to skin penetration enhancers (or any other additives), which may be considered as volatile agents, in which case their presence by weight of the nano-elements should preferably not exceed 2 wt. %.

It can be noted that some materials above defined as skin penetration enhancers may additionally serve as part of the liquid phase, provided that their combination with the main polar carriers does not affect the overall polarity of the liquid and the lack of solubility of the nano-elements therein.

### Compositions

Having reviewed the various components that may be used in the present dermatological compositions, suitable concentrations or respective proportions shall be provided below. It is to be noted that some of the components according to the present teachings can serve in more than one role. For example, some non-volatile liquids, such as aliphatic esters (e.g., dimethyl glutarate); fatty acids (e.g., lauric acid, linoleic acid, linolenic acid, myristic acid, oleic acid, palmitic acid, stearic acid and isostearic acid); fatty acid esters (e.g., ethyl oleate, glyceryl monooleate, glyceryl monocaprate, glyceryl tricaprylate, isopropyl myristate, isopropyl palmitate, propylene glycol monolaurate and propylene glycol monocaprylate); and terpenes (e.g., eugenol, D-limonene, menthol, menthone, farnesol and neridol); may also have skin penetration enhancing properties. In yet another example, some polar carriers, such as water and certain glycols and glycerols, may also assist skin-penetration, or even serve as surfactants. Thus, when referring, for instance, to the concentration of skin-penetration enhancers in the composition, the information refers only to dedicated compounds intentionally added to serve this role, excluding compounds having a different primary role in the composition.

In some embodiments, the concentration of the CSSC (or combination thereof) in the nano-elements is within the range of 0.1 wt. % to 100 wt. % by total weight of the nano-elements, (wherein 100 wt. % refers to nano-elements comprising only CSSC(s) for which the presence of a plasticizing liquid or of a surfactant is not required). In some embodiments, the concentration of the CSSC(s) is within the range of 1 wt. % to 90 wt. %, 5 wt. % to 80 wt. %, 10 wt. % to 50 wt. %, or 15 wt. % to 40 wt. % by total weight of the nano-elements.

In some embodiments, the concentration of the CSSC(s) in the dermatological composition is within the range of 0.1 wt. % to 30 wt. % by total weight of the composition, preferably in the range of 0.5 wt. % to 13 wt. %, 1 wt. % to 10 wt. %, 2 wt. % to 10 wt. %, 3 wt. % to 10 wt. %, 4 wt. % to 10 wt. %, or of 4 wt. % to 8 wt. %. In other embodiments, the CSSC(s) concentration is at least 0.1 wt. %, at least 0.5 wt. %, at least 1 wt. %, at least 2 wt. %, at least 3 wt. %, or at least 4 wt. % by total weight of the composition. In other embodiments, the concentration of the CSSC(s) is at most 30 wt. %, at most 25 wt. %, at most 20 wt. %, at most 15 wt. %, at most 13 wt. %, at most 10 wt. % or at most 8 wt. % by total weight of the composition.

In some embodiments, the polar carrier (e.g., water) is present in the dermatological composition within the range of 30 wt. % to 90 wt. %, 30 wt. % to 80 wt. %, 40 wt. % to 70 wt. %, or 30 wt. % to 60 wt. % by total weight of the composition.

In some embodiments, the concentration of the non-volatile liquid(s), if present in the nano-elements, is at most 99 wt. %, at most 90 wt. %, at most 80 wt. %, at most 70 wt. %, or at most 60 wt. % by total weight of the nano-elements.

In some embodiments, the concentration of the non-volatile liquid(s), if present in the dermatological composition, is within the range of 0.1 wt. % to 50 wt. % by total weight of the composition, preferably in the range of 0.1 wt. % to 45 wt. %, 0.1 wt. % to 40 wt. %, 0.5 wt. % to 35 wt. %, 0.5 wt. % to 30 wt. %, 0.5 wt. % to 25 wt. %, 1 wt. % to 22.5 wt. %, or of 5 wt. % to 20 wt. %. In some embodiments, the concentration of the non-volatile liquid(s) is at least 0.1 wt. %, at least 0.5 wt. %, at least 1 wt. %, or at least 5 wt. % by weight of the dermatological composition. In other embodiments, the concentration of the non-volatile liquid(s) is at most 50 wt. %, at most 45 wt. %, at most 40 wt. %, at most 35 wt. %, at most 30 wt. %, at most 25 wt. %, at most 22.5 wt. %, or at most 20 wt. % by weight of the dermatological composition. The non-volatile liquid(s) (or a combination thereof) can be included for plasticizing at a weight ratio of at least 1:200, at least 1:20, at least 1:10, at least 1:5, or at least 1:3, at least 1:1, at least 2:1, or at least 3:1, with respect to the weight of the CSSC(s) to be plasticized. In some embodiments, the weight ratio of the non-volatile liquid(s) to the CSSC(s) is of at most 100:1, at most 50:1, at most 20:1, at most 10:1, or at most 5:1.

In some embodiments, the concentration of the surfactant(s), if present in the nano-elements, is within the range of 0.1 wt. % to 50 wt. %, within the range of 1 wt. % to 50 wt. %, within the range of 5 wt. % to 50 wt. %, within the range of 10 wt. % to 50 wt. %, within the range of 15 wt. % to 45 wt. %, or within the range of 20 wt. % to 40 wt. % by total weight of the nano-elements.

In some embodiments, the combined concentration of the dedicated surfactants (including, for instance, the emulsifiers and/or hydrotropes), if present in the dermatological composition, is within the range of 0.1 wt. % to 60 wt. %, within the range of 0.5 wt. % to 60 wt. %, within the range of 1 wt. % to 60 wt. %, 5 wt. % to 40 wt. %, 6 wt. % to 30 wt. %, 7 wt. % to 25 wt. %, 8 wt. % to 20 wt. %, or 5 wt. % to 15 wt. % by total weight of the composition. In some embodiments, the combined concentration of the surfactants is at least 5 wt. %, at least 6 wt. %, at least 7 wt. %, or at least 8 wt. % by total weight of the composition. In other embodiments, the combined concentration of the surfactants is at most 40 wt. %, at most 35 wt. %, at most 30 wt. %, at most 25 wt. %, at most 20 wt. % or at most 15 wt. % by total weight of the composition.

In some embodiments, the concentration of the emulsifier(s), if present in the dermatological composition, is within the range of 0.01 wt. % to 60 wt. %, 0.1 wt. % to 50 wt. %, 0.5 wt. % to 40 wt. %, 1 wt. % to 30 wt. %, 3 wt. % to 25 wt. %, or 5 wt. % to 20 wt. % by total weight of the composition. In some embodiments, the concentration of the emulsifier(s) in the composition is at least 0.01 wt. %, at least 0.1 wt. %, at least 0.5 wt. %, at least 1 wt. %, at least 3 wt. %, or at least 5 wt. % by total weight of the composition. In other embodiments, the concentration of the emulsifier(s) in the composition is at most 60 wt. %, at most 50 wt. %, at most 40 wt. %, at most 30 wt. %, at most 25 wt. %, or at most 20 wt. % by total weight of the composition.

In some embodiments, the concentration of the hydrotrope(s), if present in the dermatological composition, is within the range of 0.01 wt. % to 60 wt. %, 0.05 wt. % to 50 wt. %, 0.1 wt. % to 40 wt. %, 0.1 wt. % to 30 wt. %, 0.5 wt. % to 25 wt. %, 1 wt. % to 20 wt. %, or 1 wt. % to 10 wt. % by total weight of the composition. In some embodiments, the hydrotrope(s) concentration in the composition is at least 0.01 wt. %, at least 0.05 wt. %, at least 0.1 wt. %, at least 0.5 wt. %, or at least 1 wt. % by total weight of the composition. In other embodiments, the hydrotrope(s) concentration in the composition is at most 60 wt. %, at most 50 wt. %, at most 40 wt. %, at most 30 wt. %, at most 25 wt. %, at most 20 wt. %, at most 15 wt. %, or at most 10 wt. % by total weight of the composition.

Alternatively, the composition is substantially devoid of a dedicated surfactant, the concentration of one or more surfactants being less than 0.1 wt. % by total weight of the composition.

In some embodiments, the concentration of the carrier-insoluble active agent, if present in the nano-elements, is within the range of 0.1 wt. % to 99.9 wt. %, within the range of 1 wt. % to 85 wt. %, within the range of 2 wt. % to 70 wt. %, within the range of 3 wt. % to 55 wt. %, within the range of 5 wt. % to 45 wt. %, within the range of 5 wt. % to 35 wt. %, within the range of 10 wt. % to 30 wt. %, or within the range of 15 wt. % to 25 wt. % by total weight of the nano-elements.

In some embodiments, the concentration of any one of the active agents, either carrier-soluble or carrier-insoluble, or of all of them if more than one, in the dermatological composition, is within the range of 0.01 wt. % to 30 wt. % by total weight of the composition, preferably in the range of 0.05 wt. % to 25 wt. %, 0.1 wt. % to 20 wt. %, 0.5 wt. % to 15 wt. %, 1 wt. % to 12.5 wt. %, 2 wt. % to 10 wt. %, 3 wt. % to 10 wt. %, or of 5 wt. % to 10 wt. %. In some embodiments, the concentration of any one of the active agents is at least 0.01 wt. %, at least 0.05 wt. %, at least 0.1 wt. %, at least 0.5 wt. %, at least 1 wt. %, at least 2 wt. %, at least 3 wt. %, or at least 5 wt. % by total weight of the composition. In other embodiments, the concentration of all the active agents or the sole one is at most 30 wt. %, at most 25 wt. %, at most 20 wt. %, at most 15 wt. %, at most 12.5 wt. %, or at most 10 wt. % by total weight of the composition.

In some embodiments, the concentration of the skin-penetration enhancer(s), if present in the dermatological composition, is within the range of 0.01 wt. % to 30 wt. %, 0.1 wt. % to 25 wt. %, 1 wt. % to 20 wt. %, 3 wt. % to 15 wt. %, or 5 wt. % to 15 wt. % by total weight of the composition.

Preferably, when the compositions are intended to be dermatological compositions, the aforesaid ingredients are approved for cosmetic or pharmaceutic use at the envisioned concentrations. For instance, they do not irritate the skin, nor lead to allergic reactions, or any other acute or chronic adverse effect. Moreover, all ingredients need be compatible one with another, such compatibility being as described above. As readily understood, this principle of compatibility, which can be affected not only by the chemical identity of the materials, but by their relative proportions according to the intended use, should preferably guide the selection of all materials necessary for the compositions disclosed herein.

Similar principles may be followed for agrochemical or industrial compositions, the ingredients being in such cases preferably of a chemical nature and at concentrations approved by the regulatory authorities governing such products.

### Method of Preparation

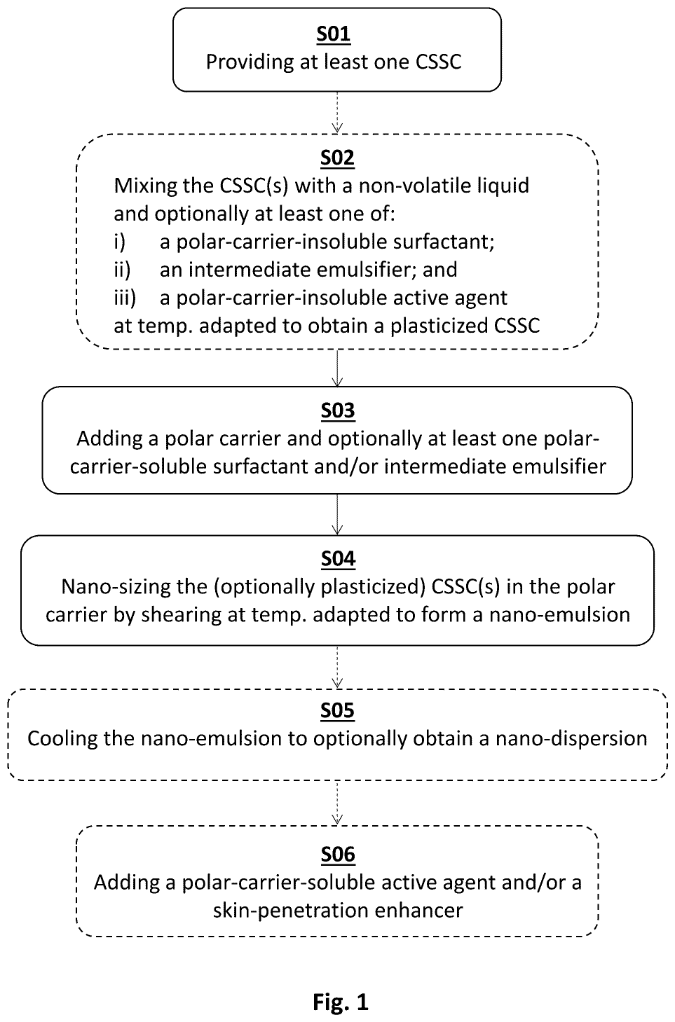

In another aspect of the present invention, there is provided a method for preparing a dermatological composition comprising nano-elements of a water-insoluble collagen-synthesis stimulating compound (CSSC), in particular of a water-insoluble collagen-synthesis stimulating polymer (CSSP), the nano-elements being dispersed as a nano-suspension in a polar liquid. The properties and characteristics of the materials used in the present method are as described above for each of the materials. The steps of the present method are briefly displayed in FIG. 1 and further detailed hereinbelow, a step having a dashed contour being optional.

In a first step (S01) of the method, at least one CSSC (e.g., at least one CSSP) is provided.

In a second step (S02) of the method, the CSSC(s) can be mixed with one or more non-volatile liquid(s), whereby the CSSC(s) undergo(es) plasticizing or swelling by the liquid. This step is optional as the viscosity of the CSSC(s) provided in S01 can be sufficiently low for further processing (e.g., 10⁷ mPa·s or less, as measured at at least one temperature between 20° C. and 80° C., and at a shear rate of 10 sec⁻¹). As the mixing step often achieves at least some plasticizing of the CSSC, it may also be referred to as a plasticizing step.

If plasticizing of the CSSC(s) is desired, the mixing with the non-volatile liquid(s) can be performed at any mixing temperature and/or mixing pressure suitable for such compounding.

The temperature at which the mixing or plasticizing is performed is typically selected according to temperatures characterizing the substances involved in the process, for instance, by taking into account a Ts, Tm and/or Tg characterizing the CSSC(s) and, optionally, a Tb of the non-volatile liquid(s) (referred to as Tbₗ). As previously detailed, a mixing temperature would suitably be higher (e.g., by at least 5° C., at least 10° C., at least 15° C., or at least 20° C.) than at least one of the characterizing temperatures of the CSSC(s) and lower (e.g., by at least 5° C., at least 10° C., at least 15° C., or at least 20° C.) than the boiling temperature of the plasticizing liquid at a pressure the mixing step is performed, though this upper limit is not essential as long as the selected mixing temperature does not significantly boil away the plasticizing liquid. Hence, in some cases, the mixing temperature can even be at Tbₗ if the step is brief enough and/or the non-volatile liquid in sufficient excess and/or the mixing performed in a chamber sufficiently sealed to limit its evaporation/favor its condensation back to the mixture.

One can readily appreciate that a change in the properties of the substance reflected by these temperatures dropping from first to second values can alternatively take place at a lower mixing temperature or a higher mixing temperature, if the pressure in a sealed chamber hosting the mixing process ensuring plasticization of the CSSC(s) were to be accordingly reduced or increased. Therefore, while in the description of a method suitable for the preparation of a composition according to the present teachings, reference can be made to specific temperatures and duration of times assuming the process is carried out under standard atmospheric pressure, such guidance should not be viewed as limiting, and all temperatures and durations achieving a similar outcome with respect to the behavior of the plasticized CSSC(s) are encompassed.

It is noted in this context, when the CSSC is a CSSP, that while the Tm and/or Tg of a polymer may set relatively clear temperatures below and above which a polymer may display a distinct behavior, this typically does not apply to the Ts. In view of their viscoelastic properties, a polymer or a plasticized polymer may remain “sufficiently solid” even at a temperature moderately higher than its formal softening point.

The mixing or plasticizing can be performed under a variety of conditions, such as elevated temperatures (i.e., 30° C. or more, e.g., at 40° C. or more, at 50° C. or more, at 60° C. or more, at 75° C. or more, or at 90° C. or more) and/or elevated pressure (i.e., 100 kPa or more, e.g., at 125 kPa or more, 150 kPa or more, 175 kPa or more, 200 kPa or more, 250 kPa or more, or 300 kPa or more), typically accelerating the plasticizing process (i.e., shortening the duration of the plasticizing period) or enabling a desired modification of a boiling temperature Tbₗ at which the non-volatile liquid might evaporate. As mixing at elevated pressure increases Tbₗ, the range of temperatures at which plasticizing could be performed can be accordingly widened. Conversely, plasticizing the CSSC using the non-volatile liquid under conditions less favorable than arbitrarily set to assess the ability of a CSSC to be plasticized by a specific agent, such as at a temperature of less than 50° C. and/or a reduced pressure of less than 100 kPa, may prolong the plasticizing process, if desired. The ability of a CSSC to be plasticized or swelled by a particular non-volatile liquid may be assessed under any one of the above temperature or pressure conditions.

Mixing of the CSSC(s) with or within the non-volatile liquid(s) by agitating the mixture can also shorten the plasticizing period, such agitating additionally ensuring that all parts of the CSSC(s) are plasticized in a relatively uniform manner, the plasticized CSSC behaving reasonably homogeneously with respect to subsequent steps of the method and results expected therefrom. If excess of the non-volatile liquid is used during the plasticizing process, it can be optionally removed before proceeding to following step(s). When the materials to be plasticized have a relatively high viscosity, the mixing step can also be referred to as compounding, and the mixing equipment can be accordingly selected.

The duration of plasticizing will inter alia depend on the CSSC(s) being plasticized, the non-volatile liquid(s) being used, the plasticizing conditions (e.g., temperature, pressure, and/or agitation), and the desired extent of plasticizing. The plasticizing period can be of at least 1 minute and at most 4 days.

In some embodiments, additional materials may be incorporated within the CSSC(s) being optionally plasticized and added during the mixing step S02. These materials, which are miscible with the CSSC(s) and typically insoluble in the polar carrier, can be at least one polar-carrier-insoluble surfactant, the surfactant(s) serving as an emulsifier, at least one polar-carrier-insoluble active agent, the active agent(s) enhancing or modifying the biological activity of the composition, or any desirable additive. The plasticizing or mixing conditions may be adapted to the presence of such additional constituents.

The mixing may be performed by any method known to the skilled artisan, such as: sonication, using a double jacket planetary mixer or a high shear mixer, etc. When the materials being mixed have a relatively high viscosity, the mixing step can be performed with a two-roll mill, a three-roll mill, an extruder and such type of equipment. In a particular embodiment, the mixing is performed by sonication.

In a third step (S03) of the method, the CSSC (optionally plasticized, and further optionally, containing at least one CSSC-miscible material such as a surfactant and/or at least one carrier-insoluble active agent) is combined with at least one polar carrier. At least one surfactant can be added to the polar carrier, if desired, at this step, the surfactant being a relatively polar emulsifier or a hydrotrope. Additional materials which are soluble in the polar carrier could also be added at this step but may equally be introduced after the following nano-sizing step.

The mixture is nano-sized in a fourth step (S04) to form a nano-suspension, whereby nano-elements of CSSC(s) optionally containing other polar-carrier-insoluble materials are dispersed in a polar liquid including the polar carrier optionally combined with other polar materials.

As the nano-sizing is typically performed by applying shear at a relatively elevated temperature, the nano-elements including the CSSC(s) are generally nano-droplets during that step and the resulting nano-suspension is a nano-emulsion. If the dispersion of the nano-droplets is achieved without adding any dedicated dispersing agent, the nano-suspension so produced may be referred to as being “self-emulsified”.

The nano-emulsion can be obtained by nano-sizing the mixture of desired materials by any method capable of shearing the CSSC (whether plasticized or not, or including additional compounds), the shearing method being selected from the group comprising: sonication, milling, attrition, high pressure homogenization, high shear mixing and high shear microfluidization. In a particular embodiment, the nano-sizing is performed by sonication.

The nano-sizing is performed at a shearing temperature that is at least equal to at least one of the first Ts, Tm and Tg of the CSSC, at least equal to at least one of the second Ts, Tm and Tg of the CSSC if plasticized, and can be, in some embodiments, at least 5° C. higher, at least 10° C. higher, or at least 15° C. higher than the highest characterizing temperature of the CSSC mix being sheared. However, while this is not essential if the shearing step is brief enough and/or the polar liquid in sufficient excess, the shearing temperature should preferably prevent significant amounts of the liquid phase being boiled away. In some embodiments, the nano-sizing temperature at which shearing is performed does not exceed the boiling temperature of the liquid phase in which the shearing is being performed or of any other liquid the evaporation of which should be prevented. Thus, the shearing temperature is generally lower than the lowest of the Tb of the polar carrier(s) (referred to as Tbe) at a pressure the nano-sizing step is performed. For instance, when the polar carrier is water, the shearing temperature can be selected to be lower than 95° C., lower than 90° C., lower than 85° C., or lower than 80° C., assuming the nano-sizing is performed at atmospheric pressure. However, if the nano-sizing were to be performed at an elevated pressure, the Tb_{c} of the polar carrier would be raised and the shearing temperature could be accordingly increased. Still illustrating with water, while its Tb is 100° C. at about 100 kPa, this boiling temperature rises to 120° C. at about 200 kPa, in which case the nanosizing temperature not to be exceeded could be of up to 115° C. As mentioned, these upper limits while preferred are not essential, as a part of the polar carrier being boiled away could be prevented at even higher temperatures if the step is brief enough, and/or the polar carrier in sufficient excess and/or the nano-sizing is performed in a chamber sufficiently sealed to limit its evaporation/favor its condensation back to the nano-suspension.

At shearing temperatures in this range of higher than Ts, Tm or Tg and optionally lower than Tbe, the CSSC(s), and in particular the CSSP(s), can completely melt and the nano-sizing process can be considered as “melt nano-emulsification”.

In some embodiments, at least 50% of the total number (D_{N}50) or volume (Dv50) of the nano-elements (e.g., nano-droplets or nano-particles) formed in this nano-sizing step have a hydrodynamic diameter of up to 200 nm, up to 190 nm, up to 175 nm, up to 150 nm, up to 125 nm, up to 100 nm, up to 90 nm, up to 80 nm, up to 70 nm, or up to 50 nm. In some embodiments, the median diameter of the nano-elements is at least 5 nm, at least 10 nm, at least 15 nm, or at least 20 nm. Advantageously, such values are applicable as determined by the volume of the nano-elements, the values determined by number being typically lower, and generally measured at room temperature.

As readily appreciated, depending on the temperatures characterizing the materials of the nano-elements and/or on the temperature at which measurements may be performed, the nano-elements can either be relatively liquid nano-droplets or relatively solid nano-particles, as the temperature is reduced. The size of the nano-particles at room temperature is commensurate with the size of the nano-droplets or slightly more compact, their median diameter not exceeding 200 nm.

In some embodiments, the size of the nano-particles or nano-droplets is determined by microscopy techniques, as known in the art (e.g., by Cryo TEM). In some embodiments, the size of the nano-elements is determined by Dynamic Light Scattering (DLS). In DLS techniques the particles are approximated to spheres of equivalent behavior and the size can be provided in term of hydrodynamic diameter. DLS also allows assessing the size distribution of a population of nano-elements.

Distribution results can be expressed in terms of the hydrodynamic diameter for a given percentage of the cumulative particle size distribution, either in terms of numbers of particles or volumes, and are typically provided for 10%, 50% and 90% of the cumulative particle size distribution. For instance, D50 refers to the maximum hydrodynamic diameter below which 50% of the sample volume or number of particles, as the case may be, exists and is interchangeably termed the median diameter per volume (Dv50) or per number (D_{N}50), respectively, and often more simply the average diameter.

In some embodiments, the nano-elements of the disclosure have a cumulative particle size distribution of D90 of 500 nm or less, or a D95 of 500 nm or less, or a D97.5 of 500 nm or less or a D99 of 500 nm or less, i.e., 90%, 95%, 97.5% or 99% of the sample volume or number of particles respectively, have a hydrodynamic diameter of no greater than 500 nm.

In some embodiments, the cumulative particle size distribution of the population of nano-elements (e.g., nano-particles) is assessed in term of number of particles (denoted D_{N}) or in term of volume of the sample (denoted Dv) comprising particles having a given hydrodynamic diameter.

Any hydrodynamic diameter having a cumulative particle size distribution of 90% or 95% or 97.5% or 99% of the particles population, whether in terms of number of particles or volume of sample, may be referred to hereinafter as the “maximum diameter”, i.e., the maximum hydrodynamic diameter of particles present in the population at the respective cumulative size distribution.

It is to be understood that the term “maximum diameter” is not intended to limit the scope of the present teachings to nano-particles having a perfect spherical shape. This term as used herein encompasses any representative dimension of the particles at cumulative particle size distribution of at least 90%, e.g., 90%, 95%, 97.5% or 99%, or any other intermediate value, of the distribution of the population.

The nano-particles or nano-droplets may, in some embodiments, be uniformly shaped and/or within a symmetrical distribution relative to a median value of the population and/or within a relatively narrow size distribution.

A particle size distribution is said to be relatively narrow if at least one of the following conditions applies:
A) the difference between the hydrodynamic diameter of 90% of the nano-elements and the hydrodynamic diameter of 10% of the nano-elements is equal to or less than 200 nm, equal to or less than 150 nm, or equal to or less than 100 nm, or equal to or less than 50 nm, which can be mathematically expressed by: (D90-D10) 200 nm and so on;
B) the ratio between a) the difference between the hydrodynamic diameter of 90% of the nano-elements and the hydrodynamic diameter of 10% of the nano-elements; and b) the hydrodynamic diameter of 50% of the nano-elements, is no more than 2.0, or no more than 1.5, or even no more than 1.0, which can be mathematically expressed by: (D90−D10)/D50≤2.0 and so on; and
C) the polydispersity index of the nano-elements is equal to or less than 0.4, or equal to or less than 0.3, or equal to or less than 0.2, which can be mathematically expressed by: PDI=σ²/d²≤0.4 and so on, wherein σ is the standard deviation of the particles distribution and d is the mean size of the particles, the PDI optionally being equal to 0.01 or more, 0.05 or more, or 0.1 or more.

The PDI information is generally readily obtained from the instrument used to measure the hydrodynamic diameter of the nano-particles.

In a fifth step (S05) of the method, the nano-emulsion may be optionally actively cooled down to a temperature below the Tm, Ts or Tg of the CSSC (or plasticized CSSC), to accelerate the relative solidification of the nano-elements, if desired in manufacturing. Such active cooling can be achieved by refrigerating the nano-suspension (e.g., placing in a coolant having a desired low temperature), by subjecting the nano-suspension to ongoing agitation to accelerate heat dissipation (and incidentally maintain proper dispersion of the nano-droplets as they cool down), or by combining both approaches. This cooling step is optional, as the nano-emulsions may be allowed to passively cool down without any agitation upon termination of nano-sizing.

In a further optional sixth step (S06) of the method, a polar-carrier-soluble active agent and/or a skin-penetration enhancer can be added and dissolved by stirring in the polar carrier. While depicted in the figure as a separate step following cooling of the nano-suspension whether active (S05) or passive, the addition of any polar-carrier-soluble material might alternatively be performed prior to or during cooling.

While in the method detailed above, some ingredients have been described as being introduced (or optionally introduced) in the composition at a particular step, this should not be construed as limiting. Some ingredients can be introduced at more than one step and can in practice be introduced step-wisely during the preparation at two separate steps and/or gradually during the performance of a step, the material being added as the step proceeds. For illustration, one or more polar-carrier soluble surfactants can be added during the shearing step, optionally different ones at the beginning and at the end of the step. Likewise, skin-penetration enhancers may, depending on the material they are selected from, be added to the non-volatile liquid during step S02, if performed, or to the polar carrier during step S03, or to the liquid polar phase of the nano-emulsion as currently described in step S06. Alternatively, such agents may be omitted, provided that the nano-elements formed by the CSSC can be transdermally delivered in an amount sufficiently effective for the sought effect.

Thus, the above-described steps can be modified, omitted (e.g., S02, S05 or S06) and additional steps may be included. For instance, the dermatological composition may comprise any additive customary to cosmetical or pharmaceutical compositions, such as moisturizers, emollients, humectants, UV-protective agents, thickeners, preservatives, antioxidants, bactericides, fungicides, chelating agents, vitamins and fragrances, or any other material adapted for their formulation in a form suiting their application to the skin. The nature and concentration of which need not be further detailed herein. The additives may be added during steps of the method already described or via new steps. Furthermore, the composition may be further treated (e.g., sterilized, filtered, etc.) in accordance with health regulations, to make it suitable for dermatological uses, in particular on human skin.

Advantageously, the present method does not seek to chemically modify its active ingredients, as might have been required for instance to jointly attach them when preparing implants. The absence of such modifications in the present compositions is expected to prevent formation of large particles that would be unable to pass the skin barrier, and/or believed to prevent an undesirable decrease in the biological activity these ingredients might provide in their native (unmodified) form, assuming they successfully penetrated the skin.

In some embodiments, the nano-elements obtained by the above methods contain less than 2 wt. %, less than 1.5 wt. %, less than 1 wt. % of a volatile organic compound (VOC) or a blend thereof by weight of the nano-elements. In particular embodiments, the nano-elements contain less than 0.5 wt. %, less than 0.4 wt. %, less than 0.3 wt. %, less than 0.2 wt. %, or less than 0.1 wt. % of a VOC (or a blend thereof) by weight of the nano-elements. In further particular embodiments, the nano-elements contain less than 0.09 wt. %, less than 0.08 wt. %, less than 0.07 wt. %, less than 0.06 wt. %, less than 0.05 wt. %, less than 0.04 wt. %, less than 0.03 wt. %, or less than 0.02 wt. % of a VOC (or a blend thereof) by weight of the nano-elements. In some embodiments, the nano-elements may contain up to 0.001 wt. % (which corresponds to 10 parts per million—ppm), up to 0.002 wt. % (20 ppm), up to 0.003 wt. % (30 ppm), up to 0.004 wt. % (40 ppm), up to 0.005 wt. % (50 ppm), up to 0.006 wt. % (60 ppm), up to 0.007 wt. % (70 ppm), up to 0.008 wt. % (80 ppm), up to 0.009 wt. % (90 ppm), or up to 0.01 wt. % (100 ppm) of a VOC (or a blend thereof) by weight of the nano-elements.

In particular embodiments, the nano-elements contain between 0 wt. % and 0.5 wt. %, between 0.0001 wt. % and 0.5 wt. %, between 0.0002 wt. % and 0.4 wt. %, between 0.0003 wt. % and 0.3 wt. %, between 0.0004 wt. % and 0.2 wt. %, between 0.0005 wt. % and 0.1 wt. %, between 0.0001 wt. % and 0.09 wt. %, between 0.0002 wt. % and 0.08 wt. %, between 0.0003 wt. % and 0.07 wt. %, between 0.0004 wt. % and 0.06 wt. %, or between 0.0005 wt. % and 0.05 wt. % of a VOC (or a blend thereof) by weight of the nano-elements.

As used herein, the term “volatile organic compound” (“VOC”) refers to an organic compound having a vapor pressure of 0.1 kPa or more, as measured at a temperature of about 20° C.

Methods for preparing nano-materials are abundantly described in the literature, the main ones suitable for hydrophobic compounds, such as the materials used in the present invention, involve solvent evaporation or solvent displacement (e.g., performed by solvent diffusion or salting-out). These methods are cumbersome and time-consuming, some to an extent being commercially unfeasible. More importantly, they typically result in solvents remaining residually (but not negligibly) trapped within the nano-particles prepared by such methods, which can be particularly critical when the solvent being used is a VOC having a high vapor pressure and low boiling point at room temperature.

As opposed to methods commonly used for preparing particles, the present invention does not requires addition of a solvent (such as a VOC) and dissolution of the CSSC (specifically CSSP) therein, requiring the subsequent removal of the solvent. When volatile solvents are later removed from particles conventionally prepared (presumably by lengthy and burdensome methods), few phenomena may be observed. The elimination of the solvent may yield porous and friable particles, moreover, as the elimination of the solvent may concurrently require the elimination of the medium in which the particles may be dispersed, this process inherently causes the collapse of the dispersion and the drying of the particles, which may then turn into aggregates or agglomerates. Once dried, in an attempt to eliminate the solvent having served for their preparation, the particles are believed to have typically a poor dispersibility as individual particles and furthermore to have a poor malleability, if any. Such particles, prepared according to methods of the art, are believed to be ineffective for the purposes of the present invention.

Thus, VOCs such as conventionally used acetone, acetonitrile, aniline, benzene, carbon tetrachloride, chloroform, cyclohexanone, dichloromethane, dioxane, dimethylesulfoxide, ethyl acetate, hexafluoroisopropyl alcohol, methylene chloride, N,N-dimethylformamide, 2-nitro-propane, 1,1,2,2-tetrachloroethane, tetrahydofuran, 1,1,2-trichloroethane and toluene are essentially absent from nano-elements according to the present teachings, either as individual solvents or as blends of two or more VOCs.

It is stressed that the afore-said low levels of VOCs are provided with respect to the nano-elements, as the liquid phase in which they are dispersed may tolerate higher amounts of such materials, as may be allowed by the resistance of the nano-elements to solubilization and/or as may be authorized by relevant regulatory authorities governing the use of compositions for any of the purposes for which the present compositions are suitable.

The nature and amount of hypothetical VOCs in nano-elements can be determined by routine analysis, e.g., by eliminating the polar carrier, and analyzing the nano-elements isolated therefrom by, e.g., Gas Chromatography. Exemplary standard methods for determining VOCs are described in ASTM D4526 or VDA 277.

In other aspects, there are provided cosmetic, therapeutic, agrochemical or industrial uses of the present compositions, enabled by the delivery to a target surface of efficacious amounts of the nano-elements comprising the CSSCs and optional active agents, according to the effect being achieved.

Cosmetic effects for improving skin appearance, achievable by the present compositions once applied onto a skin to be treated therewith, include combating collagen elastin or glycosaminoglycan breakdown, promoting collagen, elastin or glycosaminoglycan neo-synthesis, treating signs of ageing of the skin, combating wrinkles and fine lines, combating wizened skin, combating flaccid skin, combating thinned skin, combating dull, lifeless skin, combatting uneven skin pigmentation, combating lack of elasticity and/or tonicity of the skin, as well as skin bleaching or tanning.

Pharmaceutical effects, achievable by the present compositions once applied onto a skin to be treated therewith, include improving skin appearance and/or health by treating chronic skin disorders, skin lesions, restoring skin integrity, promoting wounds or surgical incisions healing, facilitating skin grafting, alleviating or treating local skin inflammation (e.g., acne), skin irritation, skin infection, skin rashes, skin pain, skin burns, skin swelling, skin allergy, as well as promoting or inhibiting hair growth, or treating dandruff.

Alternatively, the non-cosmetic effects of the present compositions are not directed to the treatment of the skin per se but to the protection of the skin to which they are applied from external factors.

The present dermatological compositions may further be used for delivery of active agents (whether CSSC-miscible and present within the nano-elements, or water-soluble and dissolved in the polar carrier where the nano-elements are dispersed), to transdermally exert their pharmaceutical effects on organs other than the skin, by delaying, alleviating, preventing or treating a transdermally treatable disorder. The additional pharmaceutical effects that can be achieved by the present compositions once delivered through the skin to the bloodstream or an organ be treated therewith, include all activities such active agents are known, or will be found, to have when delivered via the skin. Such active agents include analgesics, anesthetics, anti-addiction agents, anti-bacterials, anti-convulsants, anti-dementia agents, anti-depressants, anti-emetics, anti-fungals, anti-gout agents, anti-inflammatory agents, anti-migraine agents, anti-myasthenic agents, anti-mycobacterials, anti-neoplastics, anti-obesity agents, anti-parasitics, anti-parkinson agents, anti-psychotics, anti-spasticity agents, anti-virals, anxiolytics, bipolar agents, blood glucose regulators, cardiovascular agents, central nervous system agents, contraceptives, dental and oral agents, gastrointestinal agents, genetic/enzyme/protein disorder agents, genitourinary agents, hormonal agents (including: adrenal, pituitary, prostaglandins, sex hormones, and thyroid), hormone suppressants (including: adrenal, pituitary, and thyroid), immunological agents, infertility agents, inflammatory bowel disease agents, metabolic bone disease agents, ophthalmic agents, otic agents, respiratory tract agents, sexual disorder agents, skeletal muscle relaxants, sleep disorder agents and dietary supplements (such as: electrolytes, minerals, metals, vitamins).

These cosmetic and pharmaceutical uses encompass all activities such CSSCs are known or will be found to have when delivered to the skin, included by injection, the present invention advantageously allowing such uses to be additionally implemented by topical application of the compositions.

Agrochemical and/or industrial effects can also be achieved by the present compositions once applied onto a surface of an inert object, a plant or a pest, or the soil or the water in which plants are growing or pests are present. Such an agrochemical and/or industrial product can be used as an acaricide, an algicide, an anthelmintic, an anti-moth, an avicide, a bactericide, a chemo-sterilant, a fertilizer, a fungicide, a herbicide, an insecticide including an ovicide, an insect repellent, an insect pheromone, a molluscicide, a nematicide, a nitrification inhibitor, a pesticide, a pest repellent, a plant growth promotor, a rodenticide, a soil fertilizer, a termicide, a virucide, and a plant wound protectant.

Active agents, when added to the present nano-elements, are generally present in an amount effective to treat the intended condition by one or more application to the skin of a subject or the external surface of an object in need thereof. Considering for illustration active agents intended to treat living subjects, in other words capable of achieving a therapeutic response without significant adverse effects, then the quantity or concentration of active agent(s) is often referred to as a therapeutically effective amount. The amount deemed effective depends inter alia on the nature of the active agents, the concentration to be achieved across the skin layers, beneath the skin or at any other targeted site if the active agent is systemically delivered, the severity of the condition, the length of treatment, the regimen of administration, the absorption and excretion rates of the active agents, and like factors known to one of ordinary skill in the art of pharmacology. Appropriate amounts of active agents can consequently be in a range of values which for each active agent are known to one of ordinary skill in the art or can be optimized by conducting pertinent trials.

Preparation of the compositions for the above uses and their mode of application can be conventionally conducted and implemented, and need not be detailed herein.

### EXAMPLES

### Materials

The materials used in the following examples are listed in Table 1 below. The reported properties were retrieved from the product data sheets provided by the respective suppliers or estimated by standard methods. Unless otherwise stated, all materials were purchased at highest available purity level. N/A means that a particular information is not available.

TABLE 1

| | | | | |
|---|---|---|---|---|
| Chemical name | Commercial name | MW | Supplier | CAS No. |

Collagen-synthesis stimulating compound (CSSC)

| | | | | |
|---|---|---|---|---|
| Polycaprolactone | Polycaprolactone | 14 | Sigma- | 24980-41-4 |
| (PCL) | (PCL-14) | kDa | Aldrich ®, USA | |
| | Polycaprolactone | 25 | Polysciences, | |
| | (PCL-25) | kDa | USA | |
| | Polycaprolactone | 37 | | |
| | (PCL-37) | kDa | | |
| | Polycaprolactone | 45 | Sigma- | |
| | (PCL-45) | kDa | Aldrich ®, USA | |
| | Polycaprolactone | 80 | | |
| | (PCL-80) | kDa | | |
| Polylactic acid | Ecosoft ® 608XF | N/A | Micro | 9051-89-2 |
| (PLA) | | | Powders, USA | |
| Poly(butylene | Poly(butylene | N/A | Shaanxi Dideu | 67423-06-7 |
| succinate-co- | succinate-co-adipate) | | Medichem, | |
| adipate) (PBSA) | (PBSA) | | China | |
| Poly(lactic-co- | Poly(lactic-co- | 5-20 | Nomisma | 26780-50-7 |
| glycolic acid) | glycolic acid) (PLGA) | kDa | Healthcare | |
| (PLGA) | | | Private, India | |
| Dewaxed shellac | Dewaxed shellac type | N/A | Worlée, | 9000-59-3 |
| | 4621 | | Germany | |
| Gum rosin | Gum rosin | N/A | Guangxi | 8050-09-7 |
| | | | Wuzhou | |
| | | | Foreign Trade, | |
| | | | China | |
| Coenzyme Q10 | Coenzyme Q10 | 863.3 | Thermo Fisher | 303-98-0 |
| | | | Scientific, | |
| | | | USA | |
| Poly(ethylene-co- | Primacor ® 5990i | N/A | Dow, USA | 9010-77-9 |
| acrylic acid) | (PEAA) | | | |
| Poly(ethylene-co- | Nucrel ™ 599 | N/A | DuPont, USA | 25053-53-6 |
| methacrylic acid) | (PEMAA) | | | |

Non-volatile liquids

| | | | | |
|---|---|---|---|---|
| Dibutyl adipate | Cetiol ® B | 258.36 | BASF ®, | 105-99-7 |
| | | g/mol | Germany | |
| C₁₂-C₁₅ alkyl | Pelemol ® 256 | N/A | Phoenix | 68411-27-8 |
| benzoate | | | Chemical, Inc., | |
| | | | USA | |
| Dicaprylyl carbonate | Cetiol ® CC | 286.4 | BASF ®, | 1680-31-5 |
| | | g/mol | Germany | |
| Dibutyl sebacate | Dibutyl sebacate | 314.5 | Sigma | 109-43-3 |
| | | g/mol | Aldrich ®, USA | |
| Benzyl alcohol | Benzyl alcohol | 108.1 | Ziv Chemicals, | 100-51-6 |
| | | g/mol | Israel | |

Surfactants (emulsifiers)

| | | | | |
|---|---|---|---|---|
| D-α-tocopherol | Vitamin E TPGS | ~1,513 | Antares Health | 9002-96-4 |
| polyethylene glycol | | g/mol | Products, USA | |
| 1000 succinate | | | | |
| Poloxamer | Kolliphor ® P 407 | 9,840 to | BASF ®, | 691397-13-4 |
| (poly(ethylene | | 14,600 | Germany | |
| glycol)-block- | | g/mol | | |
| poly(propylene | | | | |
| glycol)-block- | | | | |
| poly(ethylene | | | | |
| glycol)) | | | | |
| Mixture of: | Tefose ® 63 | N/A | Gattefossé, | 9004-99-3 |
| polyoxyl 6 stearate | | | France | 111-60-4 |
| type I, | | | | |
| ethylene glycol | | | | |
| stearates and | | | | |
| polyoxyl 32 stearate | | | | |
| type I | | | | |
| Mixture of: | Olivatis ® 12C | N/A | Medolla Iberia, | N/A |
| olive oil | | | Spain | |
| polyglyceryl-6 esters | | | | |
| and polyglyceryl-6 | | | | |
| pentaoleate | | | | |
| Olive oil PEG-8 | Olivatis ® 15C | | | 103819-46-1 |
| esters | | | | |
| Mixture of: | Olivatis ® 18C | | | 85711-62-2 |
| olive oil | | | | 25383-99 |
| polyglyceryl-6 | | | | 67762-27-0 |
| esters, | | | | |
| sodium stearoyl | | | | |
| lactylate and | | | | |
| cetearyl alcohol | | | | |
| Mixture of: | Olivatis ® 20C | | | 85711-62-2 |
| olive oil | | | | 110615-47-9 |
| polyglyceryl-6 esters | | | | |
| and | | | | |
| lauryl glucoside | | | | |
| Mixture of: | Olivatis ® 21C | | | N/A |
| olive oil PEG-6 | | | | |
| esters and | | | | |
| olive oil | | | | |
| polyglyceryl-6 esters | | | | |
| Ascorbyl palmitate | Ascorbyl palmitate | 414.5 | Spec-Chem | 137-66-6 |
| | | g/mol | Industry, China | |
| Polyglyceryl-10 | Nikkol Decaglyn | N/A | Nikko | 86637-84-5 |
| pentaoleate | 5-OV | | Chemicals, | |
| Polyglyceryl-10 | Nikkol Decaglyn | N/A | Japan | 126449-40-9 |
| pentaisostearate | 5-ISV | | | |
| Oleoyl polyoxy1-6 | Labrafil ® M 1944 CS | N/A | Gattefossé, | 69071-70-1 |
| glycerides | | | France | |
| Disodium laureth | Texapon ® SB 3 KC | 410.4 | BASF ®, | 68815-56-5 |
| sulfosuccinate | | g/mol | Germany | |
| | Miconate LES(B) | N/A | Miwon | 39354-45-5 |
| Disodium lauryl | Miconate DLS(P) | 454.5 | Specialty | 13192-12-6 |
| sulfosuccinate | | g/mol | Chemicals, | |
| | | | Korea | |
| Mixture of: | Cola ®Det EQ-154 | N/A | Colonial | 26838-05-1 |
| disodium lauryl | | | Chemical, | 68439-57-6 |
| sulfosuccinate, | | | USA | 4292-10-8 |
| sodium C₁₄-C₁₆ | | | | |
| olefin sulfonate and | | | | |
| lauramidopropyl | | | | |
| betaine | | | | |
| Mixture of: | Olivoil ® glutamate | N/A | Kalichem, Italy | 67762-27-0 |
| olive oil and | | | | 8005-44-5 |
| glutamic acid | | | | 31566-31-1 |
| Polysorbate 80 | Tween ® 80 | 1,310 | Sigma- | 9005-65-6 |
| | | g/mol | Aldrich ®, USA | |
| EO/PO Block | Pluronic ® F-127 | N/A | BASF ®, | 9003-11-6 |
| Copolymer | | | Germany | |

Surfactants (hydrotropes)

| | | | | |
|---|---|---|---|---|
| Sodium dioctyl | Sodium dioctyl | 444.56 | Sigma- | 577-11-7 |
| sulfosuccinate | sulfosuccinate (AOT) | g/mol | Aldrich ®, USA | |
| Adenosine | Adenosine | 507.18 | Antai Fine | 56-65-5 |
| triphosphate (ATP) | triphosphate | g/mol | Chemical | |
| | | | Technology, | |
| | | | China | |
| Ammonium | Ammonium | 203.36 | Stepan, USA | 26447-10-9 |
| xylenesulfonate | xylenesulfonate | g/mol | | |
| Carbamide | Urea | 60.06 | Chen Shmuel | 57-13-6 |
| | | g/mol | Chemicals, | |
| | | | Israel | |

Skin-penetration enhancer

| | | | | |
|---|---|---|---|---|
| Propylene glycol | Propylene glycol | 76.09 | Chen Shmuel | 57-55-6 |
| | | g/mol | Chemicals, | |
| | | | Israel | |

Carrier-insoluble (CSSC-miscible) active agents

| | | | | |
|---|---|---|---|---|
| Retinol palmitate | Retinol palmitate | 524.86 | Xiamen | 79-81-2 |
| | | g/mol | Kingdomway | |
| | | | Vitamin, China | |
| Tadalafil | Tadalafil | 389.4 | Teva | 171596-29-5 |
| | | g/mol | Pharmaceutical | |
| Funapide | Funapide | 429.4 | Industries, | 1259933-16-8 |
| | | g/mol | Israel | |
| Neem oil | Neem oil | 720 | Manorama | 84696-25-3 |
| | | g/mol | Industries, | |
| | | | India | |
| Castor oil | Castor oil | 927 | Sigma- | 8001-79-4 |
| | | g/mol | Aldrich ®, USA | |
| DL-alpha-tocopherol | DL-alpha-Tocopherol | 430.7 | BOC | 10191-41-0 |
| | | g/mol | Sciences ®, | |
| | | | USA | |

Carrier-soluble active agent

| | | | | |
|---|---|---|---|---|
| Low molecular | LMW HA | 3 | Xi'an Lyphar | 9067-32-7 |
| weight sodium | | kDa | Biotech, China | |
| hyaluronate | | | | |

Fluorescent marker

| | | | | |
|---|---|---|---|---|
| 9-(diethylamino)- | Nile red fluorescent | 318.37 | GIHI | 7385-67-3 |
| 5H-benzo[a] | dye | | Chemicals, | |
| phenoxazin-5-one | | | China | |

Fixation fluid

| | | | | |
|---|---|---|---|---|
| Paraformaldehyde | Paraformaldehyde | N/A | N/A | 30525-89-4 |
| | (PFA) | | | |

### Equipment

Cryo-TEM: Transmission Electron Microscope (TEM), Talos 200C by Thermo Fisher Scientific™, USA, with a Lacey grid
DSC: Differential Scanning Calorimeter DSC Q2000 (TA Instruments, USA) Fluorescence microscope: BX60 microscope, equipped with a digital camera model DP73 (Olympus, Japan)
Oven: DFO-240, by MRC, Israel
Particle Size Analyzer (Dynamic Light Scattering): Zen 3600 Zetasizer (by Malvern Instruments®, United Kingdom)
Sonicator: VCX 750, by Sonics & Materials, USA
Thermo-rheometer: Thermo Scientific (Germany) Haake Mars III, with a C20/1° spindle, a gap of 0.052 mm, and a shear rate of 10 sec⁻¹.

### Example 1: Screening of Non-Volatile Liquids Adapted to Plasticize Polycaprolactone

In the present study, various candidates for non-volatile liquids (also referred to as plasticizers or swelling agents) were tested for their suitability to plasticize a collagen-synthesis stimulating compound (CSSC), and specifically, a collagen-synthesis stimulating polymer (CSSP).

Each one of the various liquids was incubated for 1 hour at 80° C. at a weight per weight ratio of 1:1 with PCL having a molecular weight of 14 kDa (PCL-14), namely 2 g of a non-volatile liquid were added to 2 g of PCL-14 in a glass vial, and the sealed vials were placed in an oven, pre-heated to the plasticizing temperature. Following incubation, the contents of the vials were mixed by hand for about 30 seconds, until clear solutions were obtained. The samples of plasticized polymers were allowed to cool down overnight (i.e., at least 12 hours) at room temperature so as to solidify. None of the liquids so tested displayed leaching out of the plasticized PCL-14, suggesting that they might be used satisfactorily at even higher weight per weight ratio.

Solid samples were then transferred to a rheometer where their viscosity was measured as a function of temperature between 20° C. and 80° C. at a ramping up temperature of 10° C./min. A reference made of unswollen PCL-14 was included in the study, this control displaying a viscosity gradually decreasing with raising temperature from about 2×10⁵ mPa·s (as measured at 50° C.) to about 2×10⁴ mPa·s (as measured at 80° C.). For comparison, unplasticized PCL having higher molecular weights, PCL-37, PCL-45 and PCL-80 to be later detailed, provided viscosities of up to about 6.2×10⁶ mPa·s, as measured at 50° C. within the range of ramping up temperatures.

In additional measurements of viscosities in this range of temperatures for samples similarly prepared at a 1:1 weight ratio, the following non-volatile liquids were found to decrease viscosity. In the case of PCL-14, all provided for a second viscosity of less than 10⁴ mPa·s (as measured at 50° C.) as compared to the first viscosity of 2×10⁵ mPa·s for this CSSC, hence affording a decrease of at least 1.5 log. These non-volatile liquids included caprylic acid, dicaprylyl carbonate, C₁₂-C₁₅ alkyl benzoate, triethyl citrate, citronellol, cyclohexane-carboxylic acid, dibutyl adipate, hinokitiol, linalool, menthol, propylene carbonate, terpinol, tert-butyl acetate, and thymol, available for instance from Sigma-Aldrich, BASF®, or Phoenix Chemical.

Based on the above-screening results, a first pair of CSSP and non-volatile liquid, namely a PCL having a molecular weight of about 14 kDa (PCL-14) and dibutyl adipate, was selected. Additional combinations of CSSCs and non-volatile liquids were similarly tested and found adapted for the preparation of dermatological compositions, as detailed in Examples 2-4.

### Example 2: Nano-Suspensions of CSSCs in an Aqueous Polar Phase

An aqueous solution containing a surfactant mixture (comprising an emulsifier and hydrotropes) was prepared as follows: 6.6 g of distilled water, 0.3 g of ammonium xylenesulfonate, 0.1 g of adenosine triphosphate and 1 g of vitamin E TPGS were placed in a 20 ml glass vial and sonicated for 10 minutes (at 40% power, operated in pulses of 7 seconds, followed by 1 second breaks), until a clear aqueous solution intended to serve as liquid polar phase for the nano-elements of CSSC was obtained.

A CSSC premix was prepared as follows: in a separate 20 ml glass vial, 3 g of PCL-14 having a native melting temperature of about 62° C. (as determined by DSC), were combined with 7 g of Cetiol® B, and the vial was placed in an oven at a temperature of 70° C.-80° C. for 1 hour until the PCL-14 was completely melted. The vial was then mixed by hand for about 30 seconds, until a clear, homogenized solution of 30 wt. % melted PCL plasticized by 70 wt. % Cetiol® B was obtained. The melting temperature of the plasticized polymer was then determined by DSC, and was found to be about 50° C., plasticizing with Cetiol® B having effectively reduced the Tm of the polymer by more than 10° C.

2 g of the CSSC premix containing the melted solution of plasticized polymer were added to the vial containing the 8 g of aqueous solution including the surfactants, and sonicated for 20 minutes (as previously described), at a shearing temperature of about 70° C., whereby a nano-emulsion containing nano-droplets of liquid polymer in an aqueous solution was obtained.

This composition is reported in Table 2A as Composition 2.1. Additional compositions were prepared according to similar procedures, each composition containing different components in different amounts, and prepared under different conditions, as specified in Tables 2A-2E. Sonication, when performed, was done as described above. The values reported in the table correspond to the concentration of each component in weight percent (wt. %) by total weight of the composition, except for the values in the CSSC premix section, which correspond to the weight percentage of each component in that particular premix. The nano-emulsions so produced were allowed to passively cool down to room temperature for 1 hour, allowing the relative solidification of the nano-droplets and. The extent of solidification, so that at room temperature the nano-droplets are sufficiently solid to constitute a nano-dispersion or conversely sufficiently liquid to constitute a nano-emulsion, can be assessed by sampling the pre-mix for a volume of the mixed materials, the isolated samples being similarly allowed to cool down without having been sheared into a liquid phase. The size of the nano-particles so produced was measured by Dynamic Light Scattering (DLS) on samples of the compositions, diluted to 1:100 in water, and the measured median diameter per volume (Dv50) and per number (D_{N}50), as well as polydispersity indices (PDI), are also presented in the tables below.

TABLE 2A

| | Composition | | | | | | | |
|---|---|---|---|---|---|---|---|---|
| Component | 2.1 | 2.2 | 2.3 | 2.4 | 2.5 | 2.6 | 2.7 | 2.8 |

CSSC premix

| | | | | | | | | |
|---|---|---|---|---|---|---|---|---|
| PCL-14 | 30 | 30 | 30 | 20 | 20 | 20 | 20 | 20 |
| Cetiol ® B | 70 | 70 | 50 | 60 | 60 | 60 | 60 | 60 |
| Tefose ® 63 | | | 20 | | | | | |
| Olivatis ® 12C | | | | 20 | 20 | | | |
| Olivatis ® 18C | | | | | | 20 | | |
| Olivatis ® 20C | | | | | | | 20 | |
| Olivatis ® 21C | | | | | | | | 20 |

| Premix | 1 hr @80° C. | 2 min @80° C. by sonication |
|---|---|---|
| preparation | in oven | |

Nano-dispersion

| PCL-14 | 6 | 6 | 6 | 4 | 4 | 4 | 4 | 4 |
|---|---|---|---|---|---|---|---|---|
| Cetiol ® B | 14 | 14 | 10 | 12 | 12 | 12 | 12 | 12 |
| Vitamin E TPGS | 10 | 15 | | | | | | |
| Ammonium | 3 | 3.6 | | | | | | |
| xylenesulfonate | | | | | | | | |
| ATP | 1 | | | | | | | |
| Tefose ® 63 | | | 4 | | | | | |
| Sodium dioctyl | | | 4 | 5 | | 5 | 5 | 5 |
| sulfosuccinate | | | | | | | | |
| Olivatis ® 12C | | | | 4 | 4 | | | |
| Olivatis ® 15C | | | | | 20 | | | |
| Olivatis ® 18C | | | | | | 4 | | |
| Olivatis ® 20C | | | | | | | 4 | |
| Olivatis ® 21C | | | | | | | | 4 |
| Propylene glycol | | 10 | | | | | | |
| Water | 66 | 51.4 | 76 | 75 | 60 | 75 | 75 | 75 |
| Nano-sizing | 20 min | | 1 min @80° C. | | | | | |
| | @70° C. | | | | | | | |
| D_{V}50 [nm] | 125.0 | 39.0 | 44.3 | 125.0 | 109.0 | 108.0 | 130.0 | 136.0 |
| D_{N}50 [nm] | 54.0 | 54.1 | 23.5 | 51.5 | 68.1 | 59.0 | 43.2 | 62.3 |
| PDI | 0.403 | 0.196 | 0.340 | N/A | 0.259 | N/A | 0.309 | N/A |

TABLE 2B

| | Composition | | | | | |
|---|---|---|---|---|---|---|
| Component | 2.9 | 2.10 | 2.11 | 2.12 | 2.13 | 2.14 |

CSSC premix

| | | | | | | |
|---|---|---|---|---|---|---|
| PCL-14 | 20 | 30 | 30 | 20 | 20 | 20 |
| Cetiol ® B | 60 | 50 | 50 | 60 | 60 | 60 |
| Tefose ® 63 | | | | 20 | 20 | 20 |
| Ascorbyl | 20 | | | | | |
| plamitate | | | | | | |
| Nikkol Decaglyn | | 20 | | | | |
| 5-OV | | | | | | |
| Nikkol Decaglyn | | | 20 | | | |
| 5-ISV | | | | | | |

| Premix | 2 min @80° C. | 4 min @80° C. by sonication |
|---|---|---|
| preparation | by sonication | |

Nano-dispersion

| PCL-14 | 4 | 6 | 6 | 4 | 4 | 4 |
|---|---|---|---|---|---|---|
| Cetiol ® B | 12 | 10 | 10 | 12 | 12 | 12 |
| Tefose ® 63 | | | | 4 | 4 | 4 |
| Sodium dioctyl | 20 | 4 | 4 | | | |
| sulfosuccinate | | | | | | |
| Ascorbyl | 4 | | | | | |
| plamitate | | | | | | |
| Nikkol Decaglyn | | 4 | | | | |
| 5-OV | | | | | | |
| Nikkol Decaglyn | | | 4 | | | |
| 5-ISV | | | | | | |
| Texapon ® SB 3 | | | | 5 | | |
| KC | | | | | | |
| Miconate DLS(P) | | | | | 5 | |
| Miconate LES(B) | | | | | | 5 |
| Olivatis ® 21C | | | | | | |
| Water | 60 | 76 | 76 | 75 | 75 | 75 |
| Nano-sizing | 1 min @80° C. | | | | | |
| D_{V}50 [nm] | 116.0 | 75.1 | 64.0 | 105.0 | 164.0 | 85.9 |
| D_{N}50 [nm] | 53.6 | 42.2 | 36.1 | 32.1 | 33.7 | 22.3 |
| PDI | 0.273 | 0.350 | 0.242 | 0.389 | 0.368 | 0.311 |

TABLE 2C

| | Composition | | | | | | | |
|---|---|---|---|---|---|---|---|---|
| Component | 2.15 | 2.16 | 2.18 | 2.19 | 2.20 | 2.21 | 2.22 | 2.23 |

CSSC premix

| PCL-14 | 20 | 20 | 20 | 20 | 20 | 30 | 20 | 70 |
|---|---|---|---|---|---|---|---|---|
| Cetiol ® B | 60 | 60 | 60 | 60 | 60 | 40 | 60 | 30 |
| Tefose ® 63 | 10 | 20 | 20 | 20 | 13.3 | 20 | 13.3 | |
| Olivatis ® 21C | 10 | | | | | | | |
| Labrafil ® M 1944 | | | | | 6.7 | 10 | 6.7 | |
| CS | | | | | | | | |
| Premix | 1 min @80° C. by sonication | | | | | | | 4 min |
| preparation | | | | | | | | @80° C. by |
| | | | | | | | | sonication |

Nano-dispersion

| PCL-14 | 4 | 4 | 4 | 4 | 4 | 6 | 4 | 28 |
|---|---|---|---|---|---|---|---|---|
| Cetiol ® B | 12 | 12 | 12 | 12 | 12 | 8 | 12 | 12 |
| Tefose ® 63 | 2 | 4 | 4 | 4 | 2.7 | 4 | 2.7 | |
| Sodium dioctyl | 1 | 1.3 | | 1 | 1.3 | 1.3 | 1 | |
| sulfosuccinate | | | | | | | | |
| Olivatis ® 15C | 19 | 18.7 | | | | | | |
| Olivatis ® 21C | 2 | | | | | | | |
| Olivoil ® | | | 20 | 19 | 18.7 | 18.7 | 18 | |
| glutamate | | | | | | | | |
| Labrafil ® M 1944 | | | | | 1.3 | 2 | 1.3 | |
| CS | | | | | | | | |
| Urea | | | | | | | 1 | |
| Vitamin E TPGS | | | | | | | | 15 |
| Ammonium | | | | | | | | 25 |
| xylenesulfonate | | | | | | | | |
| Water | 60 | 60 | 60 | 60 | 60 | 60 | 60 | 20 |
| Nano-sizing | 1 min @80° C. | | | | | | | 2 min |
| | | | | | | | | @80° C. |
| D_{V}50 [nm] | 118.0 | 86.9 | 136.0 | 89.6 | 67.7 | 37.7 | 77.1 | N/A |
| D_{N}50 [nm] | 71.5 | 50.9 | 41.2 | 55.6 | 43.3 | 34.2 | 46.9 | 109 |
| PDI | 0.176 | 0.277 | 0.429 | 0.249 | 0.190 | 0.220 | 0.232 | N/A |

Additional compositions were similarly prepared, in which PCL-14 was replaced by various CSSCs to form the core of the nano-elements. Compositions prepared using polycaprolactone of higher molecular weights, specifically, 25 kDa, 37 kDa, 45 kDa and 80 kDa, are reported in Table 2D as previously described. Compositions made using the natural CSSCs, shellac and gum rosin as core constituents are reported in Table 2E, as well as compositions prepared using the CSSPs: poly(butylene succinate-co-adipate) (PBSA) and poly(lactic-co-glycolic acid) (PLGA), and the non-polymeric CSSC, namely, Coenzyme Q10, the latter two prepared without any dedicated plasticizing non-volatile liquid. Notably, the polar carrier-insoluble active agent DL-alpha-tocopherol (vitamin E derivative), added to the CSSC premix of compositions 2.31 and 2.32, may also serve as a plasticizing agent.

TABLE 2D

| | Composition | | | | | |
|---|---|---|---|---|---|---|
| Component | 2.24 | 2.25 | 2.26 | 2.27 | 2.28 | 2.29 |

CSSC premix

| PCL-25 | | 20 | 20 | | | |
|---|---|---|---|---|---|---|
| PCL-37 | | | | | 30 | |
| PCL-45 | 10 | | | | | |
| PCL-80 | | | | 20 | | 30 |
| Cetiol ® B | 70 | 60 | 60 | 60 | 40 | 40 |
| Tefose ® 63 | 20 | 20 | 20 | 20 | 20 | 20 |
| Labrafil ® M | | | | | 10 | 10 |
| 1944 CS | | | | | | |
| Premix | 4 min | 1 min @80° C. by sonication | | | | |
| preparation | @80° C. by | | | | | |
| | sonication | | | | | |

Nano-dispersion

| PCL-25 | | 4 | 4 | | | |
|---|---|---|---|---|---|---|
| PCL-37 | | | | | 6 | |
| PCL-45 | 2 | | | | | |
| PCL-80 | | | | 4 | | 6 |
| Cetiol ® B | 14 | 12 | 12 | 12 | 8 | 8 |
| Tefose ® 63 | 4 | 4 | 4 | 4 | 4 | 4 |
| Sodium dioctyl | 5 | | 1.3 | | 1.3 | 1.3 |
| sulfosuccinate | | | | | | |
| Cola ® Det EQ- | | 5 | | | | |
| 154 | | | | | | |
| Olivatis ® 15C | | | 18.7 | | | |
| Olivoil ® | | | | 20 | 18.7 | 18.7 |
| glutamate | | | | | | |
| Labrafil ® M | | | | | 2 | 2 |
| 1944 CS | | | | | | |
| Water | 75 | 75 | 75 | 60 | 60 | 60 |
| Nano-sizing | 1 min @80° C. | | | | | |
| D_{V}50 [nm] | 157.0 | 106.0 | 124.0 | 74.2 | 35.1 | 64.8 |
| D_{N}50 [nm] | 34.5 | 33.6 | 101.0 | 35.8 | 18.2 | 49.1 |
| PDI | 0.262 | 0.371 | 0.048 | 0.239 | 0.214 | 0.156 |

TABLE 2E

| | Composition | | | | | | |
|---|---|---|---|---|---|---|---|
| Component | 2.30 | 2.31 | 2.32 | 2.33 | 2.34 | 2.35 | 2.36 |

CSSC premix

| | | | | | | | |
|---|---|---|---|---|---|---|---|
| PLA | 10 | | | | | | |
| PBSA | | 5 | 5 | | | | |
| PLGA | | | | 6 | | | |
| Shellac | | | | | 20 | | |
| Gum rosin | | | | | | 10 | |
| Coenzyme | | | | | | | 50 |
| Q10 | | | | | | | |
| Cetiol ® B | 70 | | | | | 70 | |
| Benzyl alcohol | | | | | 80 | | |
| Tefose ® 63 | 20 | 13 | 13 | | | 13 | 50 |
| Labrafil ® M | | 7 | 7 | | | 7 | |
| 1944 CS | | | | | | | |
| DL-alpha- | | 75 | 75 | | | | |
| tocopherol | | | | | | | |
| Olivatis ® 21C | | | | 94 | | | |

| Premix | 4 min | 2 min @80° C. | 1 min | 12 min |
|---|---|---|---|---|
| preparation | @80° C. by | by sonication | @80° C. by | @70° C. |
| | sonication | | sonication | in oven |

Nano-dispersion

| PLA | 1 | | | | | | |
|---|---|---|---|---|---|---|---|
| PBSA | | 0.5 | 0.5 | | | | |
| PLGA | | | | 0.6 | | | |
| Shellac | | | | | 2 | | |
| Gum rosin | | | | | | 1 | |
| Coenzyme | | | | | | | 2.5 |
| Q10 | | | | | | | |
| Cetiol ® B | 7 | | | | | 7 | |
| Benzyl alcohol | | | | | 8 | | |
| Tefose ® 63 | 2 | 1.3 | 1.3 | | | 1 | 2.5 |
| Sodium dioctyl | 5 | 1.3 | | 1.3 | 1 | 1.3 | |
| sulfosuccinate | | | | | | | |
| Cola ® Det | | | | | | | |
| EQ-154 | | | | | | | |
| Olivatis ® 15C | | | | | | | |
| Olivoil ® | | 18.7 | | 18.7 | | 18.7 | 20 |
| glutamate | | | | | | | |
| Labrafil ® M | | 0.7 | 0.7 | | | 1 | |
| 1944 CS | | | | | | | |
| Tween ® 80 | | | | | 10 | | |
| DL-alpha- | | 7.5 | 7.5 | | | | |
| tocopherol | | | | | | | |
| Pluronic ® | | | 5 | | | | |
| F-127 | | | | | | | |
| Olivatis ® 21C | | | | 9.4 | | | |
| Water | 85 | 70 | 85 | 70 | 79 | 70 | 75 |
| Nano-sizing | 1 min @80° C. | | | | | | |
| D_{V}50 [nm] | 121.0 | 68.1 | 72.9 | 49.2 | 104 | 71.3 | 61.3 |
| D_{N}50 [nm] | 38.2 | 46.1 | 51.3 | 40.5 | 89.1 | 60.5 | 45.9 |
| PDI | 0.224 | 0.155 | 0.325 | 0.114 | 0.022 | 0.086 | 0.595 |

More compositions were prepared using other non-volatile liquids instead of Cetiol® B, namely, Pelemol® 256, Cetiol® CC and dibutyl sebacate. These compositions are reported in Table 2F, as previously described.

TABLE 2F

| | Composition | | |
|---|---|---|---|
| Component | 2.37 | 2.38 | 2.39 |

CSSC premix

| | | | |
|---|---|---|---|
| PCL-14 | 10 | 10 | 30 |
| Pelemol ® 256 | 60 | | |
| Cetiol ® CC | | 60 | |
| Dibutyl sebacate | | | 40 |
| Olivatis ® 21C | 30 | | |
| Vitamin E TPGS | | 30 | |
| Tefose ® 63 | | | 20 |
| Labrafil ® M 1944 CS | | | 10 |

| | | |
|---|---|---|
| Premix preparation | 1 min @80° C. | 2 min @80° C. |
| | by sonication | by sonication |

| Nano-dispersion | | | | |
|---|---|---|---|---|
| | PCL-14 | 1 | 1 | 6 |
| | Pelemol ® 256 | 6 | | |
| | Cetiol ® CC | | 6 | |
| | Dibutyl sebacate | | | 8 |
| | Olivatis ® 21C | 3 | | |
| | Vitamin E TPGS | | 3 | |
| | Tefose ® 63 | | | 4 |
| | Labrafil ® M 1944 CS | | | 2 |
| | Olivoil ® glutamate | 20 | 20 | |
| | Sodium dioctyl | | | 2 |
| | sulfosuccinate | | | |
| | Water | 70 | 70 | 78 |
| | Nano-sizing | 1 min @80° C. | | |
| | D_{V}50 [nm] | 86.4 | 125.0 | 73.0 |
| | D_{N}50 [nm] | 54.8 | 55.5 | 37.3 |
| | PDI | 0.245 | 0.358 | 0.228 |

Compositions using non-biodegradable polymers for the formation of the cores of the nano-elements were also prepared. These compositions are reported in Table 2G, as previously described.

TABLE 2G

Composition

| | | |
|---|---|---|
| Component | 2.40 | 2.41 |

CSSC premix

| | | |
|---|---|---|
| PEAA | 10 | |
| PEMMA | | 10 |
| Cetiol ® CC | 70 | 70 |
| Tefose ® 63 | 13 | 13 |
| Labrafil ® M 1944 CS | 7 | 7 |

| | |
|---|---|
| Premix preparation | 2 min @80° C. by sonication |

Nano-dispersion

| | | |
|---|---|---|
| PEAA | 1 | |
| PEMMA | | 1 |
| Cetiol ® CC | 7 | 7 |
| Tefose ® 63 | 1.3 | 1.3 |
| Labrafil ® M 1944 CS | 0.7 | 0.7 |
| Olivoil ® glutamate | 18.7 | 18.7 |
| Sodium dioctyl | 1.3 | 1.3 |
| sulfosuccinate | | |
| Water | 70 | 70 |

| | |
|---|---|
| Nano-sizing | 1 min @80° C. |

| | | |
|---|---|---|
| D_{V}50 [nm] | 41.6 | 83.1 |
| D_{N}50 [nm] | 27.2 | 64.3 |
| PDI | 0.202 | 0.232 |

As can be seen in Tables 2A-2G, the present method is suitable to prepare nano-suspensions of nano-elements containing a CSSC, the nano-elements having Dv50 and D_{N}50 not exceeding 200 nm, these values being even lower than 100 nm for some of the compositions above reported. The PDI of the populations of nano-particles was at most about 0.4.

Samples corresponding to the above-described premixes were additionally tested for their viscosity at the end of the mixing step, when the CSSCs were at least homogeneously blended with the polar-carrier-insoluble materials, and in most case plasticized by the non-volatile liquids if present. Viscosity was determined as previously described at a shear rate of 10 sec⁻¹ over a range of temperatures between 20° C. and 80° C., and for all samples so tested the viscosity as measured at 50° C. was typically found to be of less than 10⁶ mPa·s, being generally between 10³ mPa·s and 10⁵ mPa·s, often not exceeding 5×10⁴ mPa·s, and many samples even having a viscosity of less 10⁴ mPa·s.

### Example 3: Nano-Suspensions of CSSC Including Polar-Carrier-Insoluble Active Agents

In the present example, an active agent was added to the CSSC. The water-insoluble active agents: retinol palmitate, tadalafil, funapide, neem oil and castor oil were used to exemplify the incorporation of polar-carrier-insoluble and CSSC-miscible active agents into nano-elements including the CSSC.

A CSSC/active agent premix was prepared in a 20 ml glass vial by combining 2 g of PCL-14, 1 g of retinol palmitate, 1 g of Olivatis® 12C as a surfactant and 6 g of Cetiol® B as a plasticizing non-volatile liquid. The vial was sonicated for 2 minutes (as previously described) at a temperature of about 80° C., to obtain a clear homogenized solution of plasticized CSSC. The CSSC/active agent premix was maintained in an oven at a temperature of 80° C. until mixed with the aqueous phase.

In a separate 20 ml glass vial, 7.5 g of distilled water and 0.5 g of sodium dioctyl sulfosuccinate, as an additional surfactant being a hydrotrope, were placed and sonicated for 1 minute at a temperature of about 60° C., until a clear aqueous solution intended to serve as liquid polar phase was obtained.

2 g of the hot CSSC/active agent premix were then added to the vial containing the 8 g of aqueous solution and sonicated for 1 minute at a shearing temperature of about 70-80° C., whereby a nano-emulsion containing nano-droplets of liquid PCL pre-mixed with retinol palmitate dispersed in an aqueous polar phase was obtained.

This composition is reported in Table 3 as Composition 3.1. Other compositions were prepared according to similar procedures, containing different components in different amounts, as specified in the table. Tadalafil and funapide were each first combined with the non-volatile liquid and sonicated for 2 minutes at 80° C. to obtain a clear solution containing the active agents, and then combined with the CSSC and other components as described above.

The values reported in the table correspond to the concentration of each component in weight percent (wt. %) by total weight of the composition, except for the values in the CSSC/active agent premix section, which correspond to the weight percentage of each component in that particular premix. The nano-emulsions so produced were allowed to passively cool down to room temperature for 1 hour, allowing the relative solidification of the nano-droplets and, when applicable, the formation of a nano-dispersion. Alternatively, the nano-droplets remained sufficiently liquid in the nano-emulsion at room temperature. The size and PDI values of the nano-particles so produced, measured by DLS as previously described, are also presented in Table 3.

TABLE 3

| Composition | | | | | | | | |
|---|---|---|---|---|---|---|---|---|
| 3.1 | 3.2 | 3.3 | 3.4 | 3.5 | 3.6 | 3.7 | 3.8 | 3.9 |

CSSC/active agent premix

| | | | | | | | | | |
|---|---|---|---|---|---|---|---|---|---|
| PCL | 20 | 33 | 20 | 20 | 20 | 20 | 20 | 20 | 20 |
| Cetiol v® B | 60 | 47 | 60 | 60 | 59.4 | 57 | 58 | 58 | 60 |
| Tefose ® 63 | | 13.45 | | | 13 | 13 | 13 | 13 | 10 |
| Olivatis ® 12C | | | | 10 | | | | | |
| Olivatis ® 21C | | | | | | | | | 5 |
| Labrafil ® M | | | | | 7 | 7 | 7 | 7 | |
| 1944 CS | | | | | | | | | |
| Retinol | 20 | 6.55 | 20 | 10 | | | | | 5 |
| palmitate | | | | | | | | | |
| Tadalafil | | | | | 0.6 | | | | |
| Funapide | | | | | | 3 | | | |
| Neem oil | | | | | | | 2 | | |
| Castor oil | | | | | | | | 2 | |

| Premix | 2 min @80° C. by sonication | 1 min @80° C. |
|---|---|---|
| preparation | | by sonication |

Nano-dispersion

| PCL | 6 | 9.9 | 4 | 4 | 4 | 4 | 2 | 2 | 4 |
|---|---|---|---|---|---|---|---|---|---|
| Cetiol ® B | 18 | 14.1 | 12 | 12 | 11.9 | 11.4 | 5.8 | 5.8 | 12 |
| Vitamin E | 10 | | | | | | | | |
| TPGS | | | | | | | | | |
| Ammonium | 20 | | | | | | | | |
| xylenesulfonate | | | | | | | | | |
| Tefose ® 63 | | 4 | | | 2.6 | 2.6 | 1.3 | 1.3 | 2 |
| Sodium dioctyl | | 5 | 5 | 5 | 1.3 | 1.3 | 1.3 | 1.3 | 2 |
| sulfosuccinate | | | | | | | | | |
| Olivatis ® 12C | | | | 2 | | | | | |
| Olivatis ® 15C | | | | | | | | | 18 |
| Olivatis ® 20C | | | | | | | | | 1 |
| Labrafil ® M | | | | | 1.4 | 1.4 | 0.7 | 0.7 | |
| 1944 CS | | | | | | | | | |
| Olivoil ® | | | | | 18.7 | 18.7 | 18.7 | 18.7 | |
| glutamate | | | | | | | | | |
| Retinol | 6 | 2 | 4 | 2 | | | | | 1 |
| palmitate | | | | | | | | | |
| Tadalafil | | | | | 0.1 | | | | |
| Funapide | | | | | | 0.6 | | | |
| Neem oil | | | | | | | 0.2 | | |
| Castor oil | | | | | | | | 0.2 | |
| Water | 40 | 65 | 75 | 75 | 60 | 60 | 70 | 70 | 60 |
| Nano-sizing | 2 min | 1 min @80° C. | | | | | | | |
| | @80° C. | | | | | | | | |
| D_{V}50 [nm] | 171.0 | 58.6 | 138.0 | 82.1 | 78.3 | 88.6 | 32.1 | 46.4 | 96.3 |
| D_{N}50 [nm] | 78.5 | 21.0 | 68.9 | 34.1 | 49.2 | 52.3 | 25.8 | 31.1 | 60.4 |
| PDI | 0.270 | 0.179 | 0.259 | 0.314 | 0.174 | 0.170 | 0.408 | 0.238 | 0.184 |

As can be seen in Table 3, the present method is suitable to prepare nano-suspensions of nano-elements containing a CSSC and a carrier-insoluble active agent, the nano-elements having Dv50 and D_{N}50 not exceeding 200 nm, these values being even lower than 100 nm for some of the compositions above reported. The PDI of the populations of nano-particles was at most about 0.3.

### Example 4: Nano-Suspensions of CSSC in a Liquid Phase Including a Polar-Carrier-Soluble Active Agent

An aqueous solution containing a surfactant mixture (comprising an emulsifier and a hydrotrope) was prepared as follows: 4.4 g of distilled water, 0.6 g of ammonium xylenesulfonate and 1 g of vitamin E TPGS were placed in a 20 ml glass vial and sonicated for 10 minutes (as previously described), until a clear aqueous solution including the surfactants and intended to serve as liquid polar phase was obtained.

In a separate 20 ml glass vial, a CSSC premix was prepared as follows: 3 g of PCL-14 and 7 g of Cetiol® B were combined, and the vial was placed in an oven at a temperature of 80° C. for 1 hour until the PCL was plasticized and completely melted. The vial was then mixed by hand for about 30 seconds, until a clear, homogenized solution of 30 wt. % melted PCL, swelled with 70 wt. % Cetiol® B was obtained.

2 g of the melted solution of plasticized polymer were added to the vial containing 6 g of the aqueous solution with the surfactants and sonicated for 20 minutes (as described above) at a shearing temperature of about 70° C., whereby a nano-emulsion containing nano-droplets of liquid polymer in an aqueous polar phase was obtained.

The nano-emulsion was allowed to passively cool down to room temperature for a cooling period of 1 hour, at which time 1 g of propylene glycol was added, and the contents of the vial were mixed by hand for 10 seconds. While propylene glycol was added in a relatively low amount to serve as skin permeation enhancer, its presence also contributed to the polarity of the liquid phase. Subsequently, 1 g of LMW hyaluronic acid, as the polar-carrier-soluble active agent, was added, and the vial contents were again mixed by hand for about 10 seconds until complete dissolution of the HA in the liquid polar phase.

This composition is reported in Table 4 as Composition 4.1. Additional compositions were similarly prepared, containing different components in different amounts. The values reported in the table correspond to the concentration of each ingredient in weight percent (wt. %) by total weight of the composition, except for the values in the CSSC premix section, which correspond to the weight percentage of each component in that particular premix. The size and PDI values of the nano-particles so produced, as measured by DLS as previously described, are also presented in Table 4.

TABLE 4

| | Composition | | |
|---|---|---|---|
| Component | 4.1 | 4.2 | 4.3 |

CSSC premix

| | | | |
|---|---|---|---|
| PCL-14 | 30 | 30 | 30 |
| Cetiol ® B | 70 | 70 | 40 |
| Tefose ® 63 | | | 20 |
| Labrafil ® M 1944 CS | | | 10 |

| | | |
|---|---|---|
| Premix preparation | 10 min @80° C. | 5 min @80° C. |
| | by sonication | by sonication |

Nano-dispersion

| | | | |
|---|---|---|---|
| PCL-14 | 6 | 5 | 6 |
| Cetiol ® B | 14 | 11.7 | 8 |
| Vitamin E TPGS | 10 | | |
| Kolliphor ® P 407 | | 8.3 | |
| Ammonium | 6 | 16.7 | |
| xylenesulfonate | | | |
| ATP | | 8.3 | |
| Tefose ® 63 | | | 4 |
| Labrafil ® M 1944 CS | | | 2 |
| Sodium dioctyl | | | 1.3 |
| sulfosuccinate | | | |
| Olivoil ® glutamate | | | 18.7 |
| Propylene glycol | 10 | 4.2 | |
| Water | 44 | 37.5 | |

| | | |
|---|---|---|
| Nano-sizing | 20 min @70° C. | 1 min @80° C. |
| | by sonication | by sonication |
| Cooling period | 1 hour | 12 hours |

| | | | |
|---|---|---|---|
| LMW HA | 10 | 8.3 | 0.1 |
| D_{V}50 [nm] | 56.3 | 141.0 | 74.9 |
| D_{N}50 [nm] | 41.0 | 96.7 | 52.3 |
| PDI | 0.195 | 0.212 | 0.210 |

As can be seen in Table 4, the present method is suitable to prepare nano-suspensions of nano-elements containing a CSSC and a carrier-soluble active agent, the nano-elements having Dv50 and D_{N}50 not exceeding 200 nm, the PDI of the populations of nano-particles being of at most about 0.2.

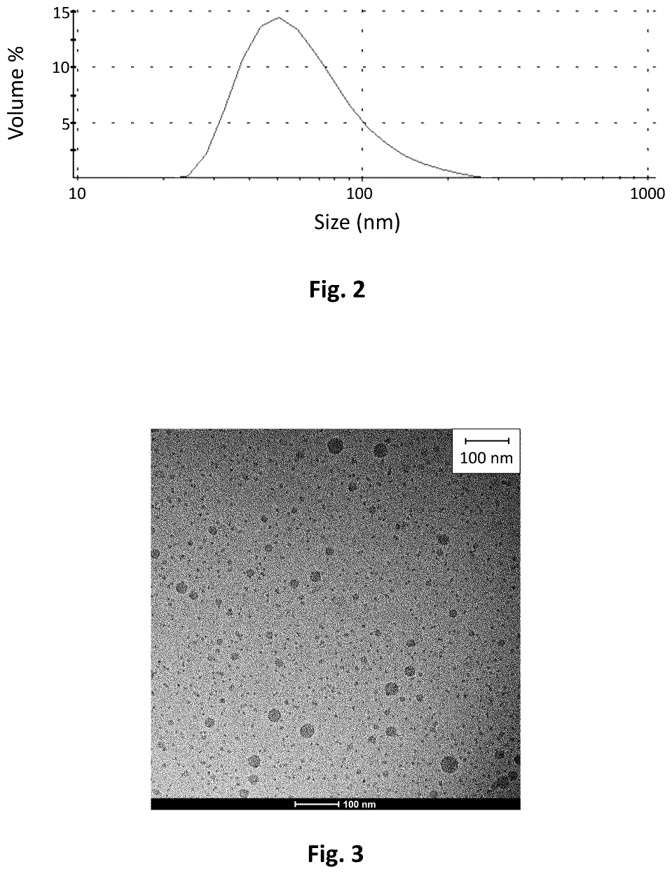

Representative results of particle size distribution in a sample of Composition 4.1, showing the percentage (per volume) of nano-particles having hydrodynamic diameters in the range of 10-1,000 nm, are presented in FIG. 2.

The size of the nano-particles of Composition 4.1 was further confirmed by microscopic TEM measurement of an image taken on a cryogenic cut of the nano-dispersion, the frozen nano-particles observed in the image having sizes in agreement with the measurements obtained by DLS. An exemplary image is shown in FIG. 3 where the nano-particles appear on the background as darker greyish globules.

### Example 5: Patch Test Protocol for Skin Irritation Analysis

The irritating effect, if any, of topically compositions according to the present teachings, such as prepared in Examples 2, 3 and 4, can be tested on skin of human volunteers by application of the formulations to be tested via a patch.

Each volunteer applies a predetermined volume of a tested composition (e.g., 0.02 ml) in a small plastic cavity (e.g., of 0.64 cm²) of an occlusive patch with a filter tissue coming in contact with the skin of the volunteer in a predetermined body are (e.g., on the back). The patch is attached to the skin area by a hypoallergenic non-woven adhesive tape and the test formulation is kept in contact with the skin for 48 hours.

The appearance of the treated area is assessed before the application of the topical compositions and 30 minutes after patch removal. Empty patches, lacking any composition, can serve as negative controls.

Skin reactions (erythema, dryness and oedema) are scored throughout the test according to the following pre-defined scoring scale:

### Erythema

0=no evidence of erythema; 0.5=minimal or doubtful erythema; 1=slight redness, spotty and diffuse; 2=moderate, uniform redness; 3=strong uniform redness; 4=fiery redness Dryness (Scaling)
0=no evidence of scaling; 0.5=dry without scaling; appears smooth and taut; 1=fine/mild scaling; 2=moderate scaling; 3=severe scaling with large flakes Oedema
−=absence of oedema; +=presence of oedema

The results obtained with any test composition are compared to those obtained on the control zone (naïve skin surface under the empty patch) and the compositions classified as: non-irritant, very slightly irritant, slightly irritant, moderately irritant, irritant, or very irritant, according to the combined effect a composition has with respect to the aforesaid prospective skin reactions.

### Example 6: Effect of the Composition on Facial Skin Appearance

The cosmetic effect of dermatological compositions according to the present teachings was tested on skin of healthy human volunteers by application of the formulations to be tested on facial skin. The parameter monitored in the present study was the number of wrinkles and small lines counted on the skin in response to various treatments. The volunteers were free of dermatological problems, irritated skin, blemishes, or such marks on test site(s) that may have impaired the study. The tested samples included topical compositions containing a predetermined concentration of CSSP and corresponding placebo compositions (prepared by the same processes) but lacking at least the CSSC (Placebo I), or also devoid of the surfactant(s) used to prepare the premix (Placebo II). The tested compositions are summarized in Table 5.

TABLE 5

| CSSC compositions | Corresponding placebo compositions |
|---|---|
| Composition 2.2, | Placebo I: |
| containing 6 wt. % PCL-14 | Ammonium xylenesulfonate: 3.6 wt. % |
| | Vitamin E TPGS: 15 wt. % |
| | Propylene glycol: 10 wt. % |
| | Distilled water: 71.4 wt. % |
| Composition 2.21 (PCL-14) | Placebo II: |
| Composition 2.28 (PCL-37) | Sodium dioctyl sulfosuccinate: 1.3 wt. % |
| Composition 2.29 (PCL-80) | Olivoil ® glutamate: 18.7 wt. % |
| each containing 6 wt. % of the | Distilled water: 71.4 wt. % |
| respective CSSCs | |

The clinical study was conducted in a double-blind manner, wherein twenty volunteers were randomly assigned to each arm of the study (i.e., twenty for each one of the CSSC compositions and twenty for each one of the respective placebo compositions). All groups applied 1 ml of their respective compositions twice-daily (morning and evening) by gently rubbing on facial skin.

The effect of the various compositions on facial skin's wrinkles was determined using Canlfield's VISIA system (by Canfield Scientific, USA), consisting of the VISIA imaging booth and VISIA software, for capturing and storing facial images using standard lighting, cross-polarized flash, and UV flash.

Measurements were taken from the targeted areas before the first application (baseline), and after one (T₁), two (T₂) and three (T₃) months of twice-daily applications of the topical compositions being tested. The results of the T₁, T₂ and T₃ timepoints were compared to the baseline values initially obtained for each volunteer, as well as to the results of the placebo arm at the same timepoints.

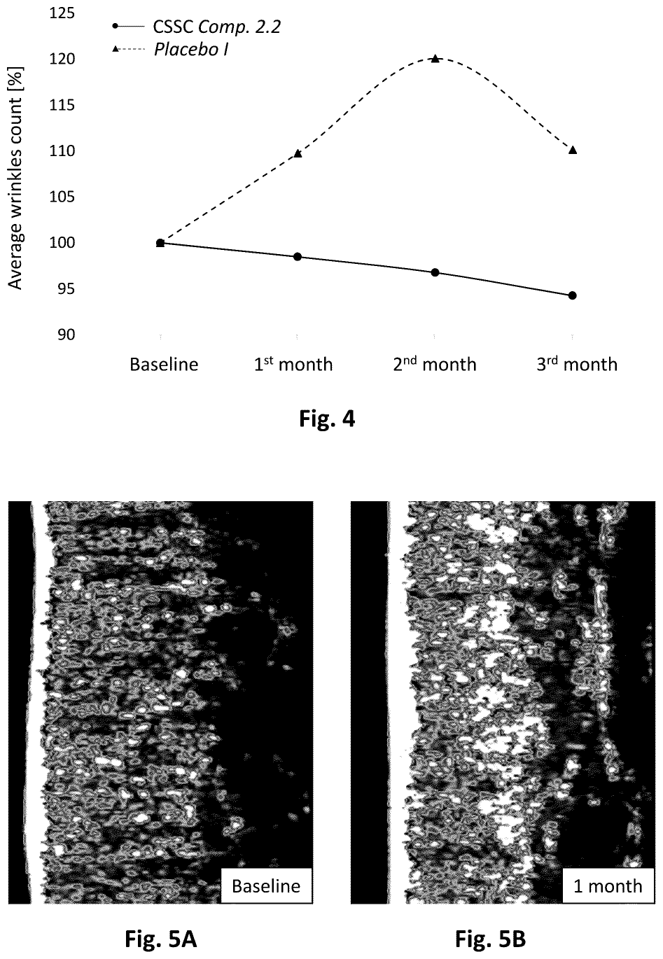

The software automatically isolated or “masked” specific areas of the face, as captured in the images, and then performed an extensive analysis of these areas to evaluate skin features such as wrinkles. The data provided by the VISIA system was displayed as “Feature Counts”, which provides a count of the number of discrete instances of the feature being evaluated (e.g., wrinkles and lines), regardless of the size or intensity of each instance. The values can be presented as the counted wrinkles on both the left and right sides of the face (“all wrinkles”), or as average values of the wrinkles counted on each of the two facial sides (“average wrinkles”). The results of all volunteers in a same group were averaged and the results of the different groups at the different time points are presented in FIG. 4.

FIG. 4 presents the changes over time in average wrinkles count in facial skin of the group treated by Composition 2.2, compared to the group treated by the corresponding placebo composition Placebo I, calculated as percentage of the average wrinkles count measured at baseline which was of about 80. As can be seen, while the group which applied the placebo composition surprisingly displayed an increase in average wrinkle count of up to 20% as normalized to the baseline, the group which applied Composition 2.2 displayed a gradually decreasing average number of wrinkles over the 3 months of the study, with a decrease of 1.5% after 1 month as compared to baseline, a decrease of 3.2% after 2 months, and a decrease of 5.7% after 3 months. This trend is reasonable, taking into account that it may take close to 3 months for CSSCs to trigger neo-synthesis of collagen to an extent that would be detectable to the naked eye.

The rapid increase in the average number of wrinkles in the control group treated with a placebo lacking the CSSC is believed to result from the presence of free surfactants in the placebo composition, the surfactant having no nano-elements to disperse. Without wishing to be bound by theory, the presence of free surfactants might adversely modify the lipid structure of skin layers, increasing accordingly trans-epidermal water loss. Such modifications may be responsible for the changes in skin appearance, its relative dryness being detected in this study by an increased number of wrinkles.

In contrast, in Composition 2.2 the surfactants are mostly bound to the CSSC (PCL), so that the amount of the free surfactants is expected to be much lower as compared to the placebo composition. When comparing the effect of Composition 2.2 to the effect of the corresponding placebo composition, it can be seen in FIG. 4 that not only did the CSSC in Composition 2.2 reduced the number of wrinkles naturally occurring in the volunteers' facial skin, but also overcame the drying effect of any free surfactants that may be present in the composition applied to the skin. Hence, the true decreasing effect of the nano-elements of CSSC on the number of wrinkles as measured for instance after 3 months might be larger than 5.7%, were the composition substantially devoid of free surfactants that could have an opposite effect on the number of wrinkles.

To confirm that the above apparently moderate effect of Composition 2.2 could result from the presence of free surfactants in the liquid phase, Compositions 2.21, 2.28 and 2.29 were similarly tested on new groups of volunteers, and compared with Placebo II lacking surfactants, applied by a corresponding control group. The results obtained after one month of application of the compositions, in terms of percent decrease as compared to respective baseline values of each group, are summarized in Table 6.

TABLE 6

| CSSC compositions | Average wrinkle count decrease |
|---|---|
| Composition 2.21 (PCL-14) | 7.8% |
| Composition 2.28 (PCL-37) | 12.1% |
| Composition 2.29 (PCL-80) | 10.7% |
| Placebo II | 0.06% |

As can be seen, after one month, the group applying the tested treatment compositions displayed a decrease in the average wrinkles count of at least 7.8%, as opposed to the group applying corresponding Placebo II, which showed an insignificant decrease of only 0.06%. Noticeably, this study established that CSSCs having a molecular weight as high as 80 kDa decreased the average wrinkle count as compared to the placebo. This supports the ability of such molecules to be transdermally delivered in a sufficiently effective amount. This trend in decreasing the number of wrinkles is expected to continue over time with ongoing application of the treating compositions, since, as known and shown with Composition 2.2, CSSCs may display a lag between the time they are applied on the skin and the time a visible effect, such as on wrinkles, can be detected following sufficient neo-synthesis of skin structural proteins. Such improvements in the efficacy of the compositions are expected at least until the stimulating activity of the CSSC allows the collagen to reach plateau levels (according to the dose of the CSSC in the composition).

A decrease in wrinkles of at least 5%, at least 10%, at least 15%, or at least 20% in the Feature Counts of a group applying the compositions of the present invention at a given timepoint as compared to the group having received the placebo composition at the same timepoint, or as compared to the respective baseline before the compositions are applied, is deemed satisfactory.

### Example 7: Effect of the Composition on Facial Skin Elasticity

The effect of the compositions of the present invention on skin elasticity was tested using Dermal Torque Meter® (DTM310) (by Dia-Stron, United Kingdom), whereby a mechanical probe exerts a predetermined torque for a predetermined length of time (“torque on”) onto the selected area of the surface of the subject's facial skin, followed by a “torque off” period, in which the force is rapidly released and the skin attempts to restore the distortion created by the torsional forces. The angular rotation of the torque disk is measured throughout this process and provided as the ratio of the “torque on” to “torque off” periods. Such measurements were made on the volunteers of the clinical study conducted as described in Example 6, using Composition 2.21 as the tested composition and Placebo II as control.

After only one month of treatment, the group applying Composition 2.21 demonstrated a statistically significant increase of 23% in elasticity as compared to baseline, whereas the volunteers that applied Placebo II showed no change to the initial elasticity of their skin. As shown in Example 6, Composition 2.21 was the relatively less effective sample in terms of reduction in the average number of wrinkles in the group compared to Placebo II. Nonetheless, this composition provided for a significant increase in skin elasticity. This trend in increasing elasticity over time is expected to continue with ongoing application of the treating composition at least until the activity of the CSSC on replenishment of skin proteins reaches a plateau.

### Example 8: Effect of the Composition on Facial Skin Hydration

The effect of the compositions of the present invention on skin hydration was measured to confirm that the changes in wrinkle count and/or elasticity, reported in previous examples, can indeed be attributed to a specific collagen-stimulating activity of the compositions and not to a change in hydration level of the skin surface.

Skin hydration analysis was performed using Corneometer® CM 825 (by Courage+ Khazaka Electronic GmbH, Germany), which measures the capacitance of the stratum corneum, using a probe capacitor effecting an electric scatter field penetrating the first layers of the stratum corneum (10-20 m). The results are reported in arbitrary units.

The capacitance variation due to skin surface hydration was measured before application of the composition (baseline) and after one month of application, as described in Example 6, with Composition 2.21 being the CSSC-containing tested composition, and Placebo II being used as control. Volunteers who applied Composition 2.21 showed a minor decrease of about 5% in hydration levels, while volunteers who applied Placebo II showed a small increase of about 3.9%.

These results indicate that neither the CSSC-containing composition nor the control composition significantly affect the skin hydration level. Hydration levels are not expected to change after continued application of the present compositions, such values reaching saturation relatively rapidly and only fluctuating as a result of climatic conditions. More importantly, these results support that the effect of the present compositions on reducing the number of wrinkles and/or increasing skin elasticity can be specifically attributed to the nano-elements containing the CSSC and do not result from an effect of the liquid phase on skin properties.

### Example 9: Effect of the Composition on In Vivo Collagen Production within Facial Skin

To confirm that the efficacy of the present compositions stems inter alia from the nano-elements of CSSCs, their ability to be transdermally delivered, and their ability to fulfil their biological role of stimulating neo-synthesis of skin proteins, collagen levels in facial skin were measured before and after application of the compositions. Measurements were made using DermaLab Combo (by Cortex Technology, Denmark), a skin analysis device whereby an ultrasound probe capable of high frequency and resolution analysis is used by passing the probe on the targeted area in the face. The output included an image of varying colors and intensities showing inter alia spots of collagen presence, the intensity of the spots corresponding to the collagen being recorded in arbitrary units.

The assessment was performed before and following application by volunteers for one month of Composition 2.21, as compared to the corresponding placebo composition Placebo II according to the methodology described in Example 6.

An increase of up to 37% in collagen formation was observed after one month of application of Composition 2.21 in the volunteer group, as compared to a minor decrease of 1.6% after a month of applying the corresponding Placebo II composition on the control volunteer group.

The changes in collagen levels may be also visually observed in the images produced following the above measurements. FIG. 5A represents the output of the measuring instrument, showing the collagen levels of the skin of an exemplary subject before application of the tested composition. The existing collagen reservoirs within the skin are visible as the small white rounded areas in FIG. 5A. FIG. 5B represents the output of the measuring instrument, showing the collagen levels of the same subject measured after one month of applying Composition 2.21. It can easily be seen that the collagen levels of this subject have dramatically increased, as evidenced by the increased number and surface occupied by the white areas showing collagen presence (as resulting from collagen-synthesis stimulating activity of the CSSC) in FIG. 5B.

### Example 10: Appearance of the Facial Skin Following Application of the Composition

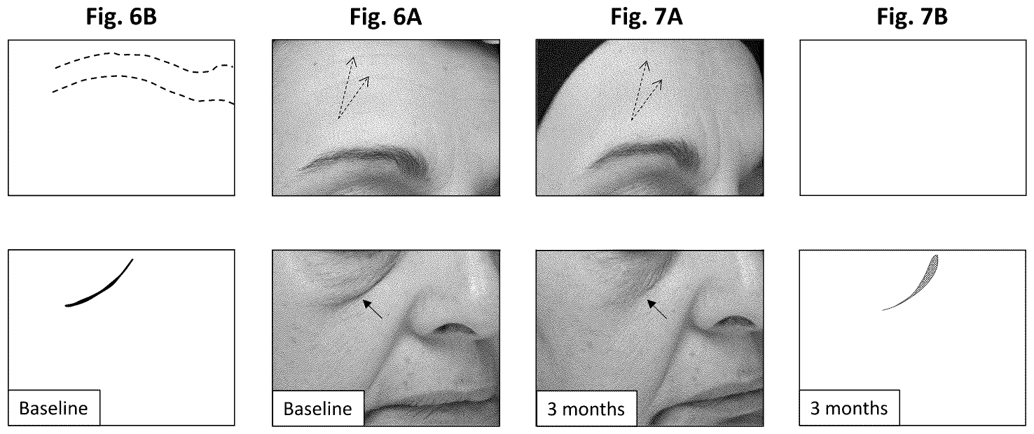

Pictures taken of a selected volunteer to visually present the effect of Composition 2.2, compared to the baseline, are shown in FIGS. 6A and 7A, and schematically illustrated in FIGS. 6B and 7B. Each of these figures is provided in two parts corresponding respectively to an upper and lower portion of the face, above and below the eyes of the photographed volunteer.

FIG. 6A shows a facial image of the volunteer, prior to application of any composition (“baseline”), where wrinkles are clearly visible in the volunteer's forehead (indicated by dashed arrows in FIG. 6A, and schematically drawn as dashed lines in the upper part of FIG. 6B), and a deep wrinkle bordering a wizened area below the eye (indicated by a full arrow in the bottom part of FIG. 6A, and schematically drawn as a full line in the bottom part of FIG. 6B).

The facial image of the same volunteer taken after 3 months of applying Composition 2.2 twice a day, as described above, is shown in FIG. 7A. The forehead wrinkles are no longer visible after 3 months, and the wrinkle bordering the wizened area under the eye became less deep and flatter (schematically drawn in the bottom part of FIG. 7B).

### Example 11: Penetration of Nano-Elements into Biological Tissue

The distribution of nano-elements of the present compositions in various skin layers was tested ex vivo by applying onto the skin of pigs' ears nano-elements including a fluorescent dye (Nile red) allowing histological monitoring of their presence within the layers of the tissue onto which they were applied.

### Tested Compositions:

The following test and control compositions were prepared and used as set in Table 7.

TABLE 7

| Tested composition | No. of repeats |
|---|---|
| (1) Saline, with no fluorescent marker | 2 |
| (2) Composition 2.21, modified to contain Nile red | 6 |
| in PCL core | |

Tested composition (2) was prepared by adding 2 mg of Nile red fluorescent marker during the preparation of the CSSC premix, as described in Example 2.

The viscosity of tested composition (2), containing a plasticizing agent, was 2×10⁵ mPa·s, as measured at a temperature of 32-37° C., indicating a lower viscosity as compared to unplasticized CSSC (2×10⁵ mPa·s, measured at a temperature of 50° C., as reported in Example 1).

Both tested compositions were stored in closed containers at room temperature until they were used.

### Testing Procedure:

The compositions were tested on two freshly harvested pig ears, collected on the same day, and kept fresh and cooled till use. The ears were washed with excess saline solution and wiped dry. They were then immobilized for the duration of the study by taping them onto a glass surface, their inner side facing down. Each ear was marked on its outer side with 6 circles having a diameter of about 1 cm.

25 microliters of each one of the samples were applied onto the skin near the center of the marked circle, each tested composition being applied in two or more separate circles for repeats. The ears with the applied test samples were then placed in an oven for an incubation time of 20 hours at a temperature of 32° C., to simulate the pigs' body temperature.

Following this incubation, the ears were punched with a metal punch having a diameter of about 8 mm to extract tissue samples of each circle onto which the different compositions were applied. Each punched-out skin sample was placed in a 50 ml plastic cup and immediately topped with 5 ml of a 4% paraformaldehyde solution, used as a fixation fluid to preserve the tissue sample and reduce leaching out of the dye. The skin samples were kept in the paraformaldehyde solution for at least 48 hours at 4° C. for complete fixation.

### Tissue Collection, Fixation and Slide Preparation:

The tissue samples were trimmed, placed in an embedding cassette and processed for paraffin embedding by immersing each sample in various reagents (formaldehyde, then water, then ethanol at various grades and finally xylene) for periods of 2-10 minutes at temperatures ranging from 37-45° C., to remove traces of the paraformaldehyde and any other contaminants. Subsequently, the samples were immersed in paraffin 3 times for 5-10 minutes each time, at a temperature of 65° C.

Sections of each processed sample were prepared using a frozen section microtome and 4 μm thick sections were placed on glass slides.

### Analysis and Results:

The tissue sections were evaluated by an expert for the presence and intensity of the marker using a fluorescence microscope at objective magnification of ×10.

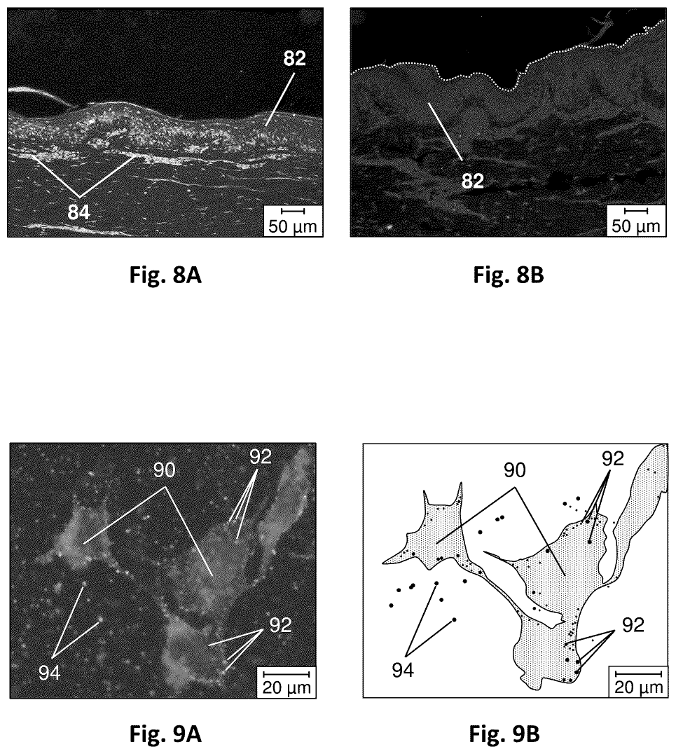

The group of samples treated with fluorescently-marked Composition 2.21 showed positive cells, i.e., being labelled with the fluorescent marker, in all of the six tested tissue samples. This is shown in FIG. 8A, presenting a histological picture of a cross-section of one of the tissue samples, taken by the microscope camera at a magnification of ×10. In FIG. 8A, the penetration of the composition into the epidermis 82 is demonstrated as an area with bright dots, indicating the presence of the fluorescent marker in this skin layer. Four of the samples also showed penetration into the dermis 84, as indicated by fluorescence in this inner part of the tissue.

FIG. 8B, showing a sample image of a cross-section from the control group treated with saline displays very low fluorescence of the skin layers, believed to be attributed to auto-fluorescence expressed by the skin tissue itself. This phenomenon is expressed in lower fluorescence levels compared to FIG. 8A, as can be expected. For convenience, the skin border, poorly-discernable in the control, is marked with a dotted white line.

It is stressed that these findings can be unambiguously attributed to the behavior of the nano-elements of the tested composition, and do not result from the independent penetration of the fluorescent Nile red marker. This was established in a control experiment in which samples containing: i) the marker in water; or ii) the marker in a solution containing water and the surfactants of Composition 2.21; were separately applied to marked circles on pig ear skin. The study was performed as described above for the tested compositions. No penetration of the fluorescent marker was observed with any of the control compositions lacking the nano-elements, indicating that the marker on its own, or even surrounded by surfactants, cannot penetrate into the skin.

Hence, this ex vivo experiment demonstrates the transdermal penetration of CSSC materials having a molecular weight significantly greater than the generally acknowledged size limit of about 0.5 kDa, the CSSC used in the tested Composition 2.21 being a polycaprolactone polymer having an average MW of about 14 kDa. Moreover, the reduced viscosity of the plasticized nano-elements contributed to their malleability, presumably aiding their penetration into the skin.

### Example 12: Cellular Penetration of Nano-Elements

The cellular penetration of nano-elements of the present compositions was tested in vitro by incubating a composition including a fluorescent dye within its nano-elements in a cell culture, allowing monitoring by fluorescence microscopy. The penetration of the nano-elements was assessed in cortical neuronal cells isolated from newborn rodents.

### Materials

The materials used in the following study are listed in Table 8 below.

TABLE 8

| | | Catalogue/ |
|---|---|---|
| Commercial name/acronym | Supplier | CAS No. |
| B-27 ™ Supplement (50X), serum free | Gibco ™, USA | 17504044 |
| GlutaMAX ™ | Gibco ™, USA | 35050038 |
| Neurobasal ™ Medium (NB) | Gibco ™, USA | 21103049 |
| TrypLE ™ Express Enzyme | Gibco ™, USA | 12604013 |
| Gentamicin sulfate | Alfa Aesar, USA | J62834.1 |
| HyClone ™ Hank's 1X Balanced | Cytiva, USA | SH30030.02 |
| Salt Solutions (HBSS) | | |
| Hyclone ™ Defined Fetal Bovine | Cytiva, USA | SH30070.03 |
| Serum (FBS) | | |
| (4-(2-hydroxyethyl)-1- | Biological | 7365-45-9 |
| piperazineethanesulfonic acid) | Industries, | |
| (HEPES) | Israel | |
| Poly-L-lysine (PLL) | Sigma Aldrich, | 25988-63-0 |
| | USA | |
| Dulbecco Phosphate-Buffered | Sartorius, | 02-023-1A |
| Saline (DPBS) | Germany | |

### Study System

All solutions and equipment used in the cell culture study were sterile, all manipulations being performed in a laminar flow cabinet. All incubations were performed in a tissue culture incubator maintained at 37° C., with CO₂ at a concentration of 5% and 95% relative humidity. The following solutions were used during the study: Brain neuronal culture (BNC) medium containing Neurobasal™ Medium including 5 vol. % of FBS, 2 vol. % of B-27 Supplement, 1 vol. % of GlutaMAX™ and 30 ppm of gentamicin sulfate; a supplemented NB (SNB) medium corresponding to the BNC medium excluding FBS; and a Dissociation solution containing 2 vol. % of HEPES in HBSS.

A 24-wells tissue culture plate was pre-treated as follows to receive the cells to be cultured therein. A round glass microscope cover slip was placed at the bottom of each well. 0.3 ml of a poly-L-lysine (PLL) solution at a concentration of 0.1 mg/mL was added to each well and the plate incubated at 37° C. for 3 hours while gently shaking to ensure full coating of the cover slips by the solution. The PLL was then aspirated, and the wells rinsed with NB medium to eliminate residual PLL. 0.5 ml of the BNC medium were then added to each rinsed well and the plate including the cover slips pre-treated to promote the adhesion of the cells to be cultivated therewith maintained in the incubator until used.

### Cell Culture Preparation

The cells were prepared according to the following procedure:
1. Tissue collection: two newborn C57 black mice were terminated by cervical cuts, and their brains were rapidly dissected using disinfected standard scissors. The brain cuts were collected in a 35 mm petri dish filled with 5 ml of the Dissociation solution and kept on ice.
2. Dissociation: the contents of the petri dish were then transferred into a 15 ml centrifugation tube, the brain cuts were allowed to settle, followed by the removal and discarding of the supernatant. Dissociation solution was added to the isolated brain pieces to a total volume of 4 ml, to which were added 1 ml of TrypLE™ Express Enzyme so as to induce the separation of the neuronal cells from other tissues. The tube and its contents were maintained for 20 minutes at 37° C. in an incubator while being gently shaken. The supernatant was then carefully removed and replaced with 1.2 ml NB medium.
3. Trituration: the tissues were subsequently triturated to yield single cells by pipetting up and down through glass Pasteur pipettes having decreasing opening size to yield a homogeneous solution containing single cells. The obtained cell suspension was centrifuged for 5 minutes at 500 RPM, the supernatant was discarded and the pellet resuspended in 2 ml of BNC medium. The cells resuspended for a rinsing cycle were again precipitated by centrifugation for 5 minutes at 500 RPM.
4. Resuspension: the supernatant was carefully removed, and the rinsed cells were resuspended in 1.5 ml BNC medium and the cell concentration determined using a counting chamber and light stereo microscope at suitable magnification (Stemi 200-C by Olympus).
5. Culture: the suspended cells were added to wells of the pre-treated 24-well plate, including poly-L-lysine coated coverslips at the bottom of the wells, at a volume ensuring the seeding of about 80,000 cells per well. The plate comprising in each well 0.5 ml of the BNC medium and 80,000 neuronal cells was maintained in the incubator for 3 days. One day before the addition of the tested composition, the medium was aspired and replaced by 1 ml per well of SNB medium.

### Tested Composition

The composition used for the present assay was prepared as described in Example 2, with the addition of 2 mg of the fluorescent marker Nile red during the preparation of the CSSC premix. The final composition contained: 4 wt. % of PCL-14, 1.3 wt. % of Labrafil® M 1944 CS, 2.7 wt. % of Tefose® 63, 12 wt. % of Cetiol® B, 0.004 wt. % of Nile red, 1.3 wt. % of sodium dioctyl sulfosuccinate, 18.7 wt. % of Olivoil® glutamate and 60 wt. % of water.

The nano-dispersion was then sterilized by passing through a syringe equipped with a 100 nm filter and was stored in closed containers at room temperature until used.

### In-Vitro Study

After 3 days of incubation of the neuronal cells in BNC medium and 1 day of incubation in SNB medium, the tested composition was added to the wells including the pre-treated cover slips to which the cells adhered during the incubation period. Samples were added to the wells in two repeats, 1 μl being added to yield a concentration of 0.1 vol. % and 10 μl being added to yield a concentration of 1 vol. %. The plate was then incubated at 37° C. for 20 minutes to allow penetration of the nano-elements into the neuronal cells.

At the end of the incubation with the tested composition, the cover slips were retrieved from their respective wells, rinsed with DPBS and placed in a 35 mm petri dish with 2 ml of DPBS, so that the cells could be studied under a microscope while still alive (e.g., within one hour of being transferred to the DPBS). The presence of the dye within the cells was determined by fluorescence microscopy (using BX43 Olympus microscope equipped with a fluorescence filter and measured at a wavelength of 594 nm using cellSens software).

### Results

FIG. 9A shows cortical neuronal cells 90, and nano-elements containing the dye 92 can be seen within the neuronal cells, thus confirming the ability of the present nano-elements to penetrate into cells. Nano-elements 94 are also visible outside the cells, as can be expected considering that the composition containing them was applied externally onto the cell culture. FIG. 9B schematically depicts the cells of FIG. 9A, to better illustrate the cells, their outer membranes and the disposition of the nano-elements with respect to such biological barriers.

### Example 13: Effect of the Compositions on Wound Healing

The following study was conducted to determine the efficacy of a composition according to the present teachings on wound healing in pigs, following its topical application to wounds on the pigs' skin for 15 days during which the wounds were observed and assessed for healing.

### Study System

The study followed the Guidance for Industry “Chronic Cutaneous Ulcer and Burn Wounds—Developing Products for Treatment” with regards to the pre-clinical model and ISO10993-6 for histology evaluation.

Three young adult healthy male pigs (Danish Landrace X-Large White Crossbred), each initially weighing between 13 and 17 kg were used in this study. They were individually ear marked and housed in stainless steel walled pens, in a controlled environment at a temperature of 17-24° C., with a relative humidity (RH) of 30-70%, a 12:12 hour light: dark cycle and 15 air changes/h in the study room. They were provided a commercial pig diet and had free access to fresh drinking water during acclimation and throughout the entire study duration. The study was initiated following 5 days of acclimatation.

### Wound Formation Surgery

The acclimated pigs were anesthetized by an isoflurane/oxygen mixture, which was delivered through a facemask, and the animals were kept under anesthesia for the duration of the surgery. Six full skin and fascia incisions of 2 cm×2 cm were made, each using a separate sterile set of surgery tools, in 6 different locations on the back of each pig (half on each side). Each incision was given a unique identification number, treated as detailed in the following section and was then covered with a sterile transparent film dressing, 3M™ Tegaderm.

Following the surgery, and the application of the first treatment dose, the pigs were returned to their pens for recovery and observation. For 4 consecutive days, they received 0.5 ml of antibiotic (Marbofloxacin 10%, commercially available as Marbocyl® by Vetoquinol, USA) and 0.5 ml of painkiller (Metamizole, commercially available as Calmagine® by Vetoquinol, USA), administered by intramuscular (IM) route at the animal neck.

Tested Compositions and their Application

The application of test compositions was initiated immediately after the surgical formation of the wounds, which was defined as the first dose of the first study day. The experimental groups comprised in the study and the regimen of administration are presented in Table 9. Before use in the present study, all tested compositions were sterilized, G1-G2 by exposure to 25-40 KGy Gamma radiation and G3 by autoclave.

TABLE 9

| Group | | Volume of | |
|---|---|---|---|
| No. | Tested composition | treatment | Regimen |
| G1 | Composition 2.39, as listed in | 300-700 μl | Twice a day |
| | Table 2F | | with 10 hours |
| G2 | Placebo: a composition comprising | | apart in all |
| | the polar carrier and surfactant | | study days |
| | external to the nano-elements | | |
| | of Composition 2.39 (i.e., 99 | | |
| | wt. % water and 1 wt. % sodium | | |
| | dioctyl sulfosuccinate) | | |
| G3 | Un-treated: saline | | |

Before the application of the tested compositions, the pigs were anesthetized with a 3% isoflurane/97% oxygen mixture as described for the initial surgery and the first dose. Whilst connected to the anesthesia, each pig was placed on its belly (prone position), the dressing was removed, and the entire wound area was cleaned with saline. Each one of the three tested compositions was dripped into a separate wound, one on each side of the back of the animal, so as to have two applications of each composition per animal. Each wound was thereafter covered with a new transparent sterile dressing. The same dermal test site was used for the same composition in each application per animal throughout the entire study. The dermal test sites varied between animals, for instance, if tested composition of group G1 was applied onto an incision near the neck of a first pig, its application on a second pig occurred on a farther away incision closer to the pig's tail. The variation in the dermal test sites across different pigs allowed to obtain wound healing results of the same composition at various dermal sites.

### Observations and Examinations

General clinical signs were recorded once daily during the acclimation period and during the treatment period. Animal body weight was measured at the beginning of acclimation, on study day 1 (prior to surgery), on study day 7, on study day 10 and at the end of the study (day 15). The animals naturally gained weight during the duration of the study, suggesting that none of the wounds and their treatment affected their appetite.

Once a day, while the animals were under anesthesia and the dressing removed, a representative photo of the wound was taken using a specialized camera and 3D scanner adapted for wound and skin assessment (SilhouetteStar by Aranz Medical, New Zealand). Wound inspection and recording of changes were also performed at that time. The wounds were then treated as planned, covered with a new sterile dressing, and the animals returned to their cages for recovery.

The wounds were evaluated prior to the daily administration of the tested compositions, using the score detailed in Table 10.

TABLE 10

| Observation | Definition/Gradation |
|---|---|
| Erythema | 0 = Absent |
| (redness) | 1 = Mild |
| | 2 = Moderate |
| | 3 = Severe |
| Edema (swelling) | 0 = Absent |
| | 1 = Mild |
| | 2 = Moderate |
| | 3 = Severe |
| Exudate/Drainage | 0 = Absent |
| | 1 = Serous (clear to yellow-tinged) |
| | 2 = Sanguineous (blood-like) |
| | 3 = Serous-Sanguineous (mixture of clear fluid |
| | and blood-like fluid) |
| | 4 = Purulent (ex. White-tan, creamy, thick) |
| Exudate/Drainage | 0 = Absent |
| Amount | 1 = Mild |
| | 2 = Moderate |
| | 3 = Heavy |
| Granulation tissue | 0 = 0 to 33% |
| filling of the | 1 = 34 to 66% |
| wound bed | 2 = 67 to 90% |
| | 3 = 91 to 100% |
| Infection (based on | Yes |
| above findings) | No |
| Scab | 0 = 0 to 33% |
| | 1 = 34 to 66% |
| | 2 = 67 to 90% |
| | 3 = 91 to 100% |
| Odor recording | Yes |
| | No |

First, it should be generally noted that no bad odor was perceived, nor infection of the wounds observed, during the entire duration of the study with any of the treatment groups. Such observations were reinforced by a lack of exudate from wounds of all groups.

With respect to erythema, mild redness was observed in the area surrounding the wounds from day 3 to about day 7 of the study, with a peak average value of about 1.0 and 1.2 on day 6, for the G2 and G3 control groups respectively. On the same day, wounds of the G1 group, treated with the present composition, displayed a lower average erythema score of 0.83, the erythema completely disappearing from day 8 onward for all three groups. Mild swelling was observed between day 4 and day 6, but totally disappeared in all treated groups from day 7 and on. Wounds of the G1 group displayed their highest swelling on day 4 and even then, their average edema score was only 0.5. Granulation tissue filling of the wound bed started to be observed from day 8 of the study, while scabs were first observed on day 10. Finally, the wound area was averaged on each day and for each group, and the measurements made from day 2 onward were normalized to day 1, set to display 100% of wound area or 0% of wound closing. It is to be noted, that while the incisions made in the skin were intended to form wounds of about 4 cm² with a perimeter of about 8 cm, the natural stretching of the skin surrounding the excised area resulted in larger wounds of about 8 cm² initial area with a perimeter of about 11 cm. Wounds of the G1 group steadily closed over time, with an average daily decrease in wound area of about 5.5% so that at the end of the study the average area of G1 wounds was about 22% of their original size. In parallel, the perimeter of the wound also decreased by about 53%, the residual perimeter of the wound at the end of the study being on average about 47% of their original size. Thus, wounds treated with a composition according to the present teachings achieved a closing of about 78% over 15 days of study duration for full-thickness excision wounds having an initial size of about 8 cm² and depth between 3 mm and 6 mm, depending on the localization of the wound.

### Termination

At the end of the study, the animals were euthanized by pentobarbitone sodium (>100 mg/kg intraperitoneally).

### Example 14: Use of the Compositions for the Transdermal Delivery of Active Agents

The following pharmacokinetic study can be conducted to determine the effect of a composition according to the present teachings on the delivery of an active agent to the blood stream of a living animal following its topical administration to the skin of the animal.

The active agent can be CSSC-miscible and disposed within the present nano-elements, such as done in compositions of Example 3. The active agent used in the present study should be unambiguously detectable in blood samples of the study animals (e.g., rodents or larger mammals). Tadalafil, such as found in the nano-elements of Composition 3.5, can be measured, e.g., by liquid chromatography with tandem mass spectrometry (LC-MS-MS) and can therefore be suitable.

In the present study, a single effective dose of the composition containing the active agent can be applied onto the shaven backs of the study animal (e.g., rats or pigs), under anesthesia. The animals, which can be divided into groups, are bled at pre-defined time points (e.g., 15 min, 30 min, 1 hr, 2 hrs, 4 hrs, 8 hrs, 12 hrs, 24 hrs and 48 hrs after application), each group (typically of 3 animals each) participating in no more than 3 samplings. The blood samples can be further manipulated as needed to enable measuring the level of the active agent, such as centrifuging and collecting the separated plasma.

A pharmacokinetic analysis can be done on the obtained plasma samples, in order to quantify the amount of active agent in the blood samples, or serum or plasma obtained therefrom, so as to determine the efficacy of the present compositions for systemic delivery.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the present disclosure has been described with respect to various specific embodiments presented thereof for the sake of illustration only, such specifically disclosed embodiments should not be considered limiting. Many other alternatives, modifications and variations of such embodiments will occur to those skilled in the art based upon Applicant's disclosure herein. Accordingly, it is intended to embrace all such alternatives, modifications and variations and to be bound only by the spirit and scope of the disclosure and any change which come within their meaning and range of equivalency.

In the description and claims of the present disclosure, each of the verbs “comprise”, “include” and “have”, and conjugates thereof, are used to indicate that the object or objects of the verb are not necessarily a complete listing of features, members, steps, components, elements or parts of the subject or subjects of the verb. Yet, it is contemplated that the compositions of the present teachings also consist essentially of, or consist of, the recited components, and that the methods of the present teachings also consist essentially of, or consist of, the recited process steps.

As used herein, the singular form “a”, “an” and “the” include plural references and mean “at least one” or “one or more” unless the context clearly dictates otherwise. At least one of A and B is intended to mean either A or B, and may mean, in some embodiments, A and B. A “material” that may be present in the composition alone or in combination with other materials of the same type can be referred to as “material(s)”; CSSC(s), CSSP(s), polar carrier(s), non-volatile liquid(s), surfactant(s), active agent(s) and the like, respectively indicating that at least one CSSC, at least one CSSP, at least one polar carrier, at least one non-volatile liquid, at least one surfactant, at least one active agent, and so on, can be used in the present methods or be included in the composition or satisfy the recited parameter or suitable range thereof.

Unless otherwise stated, the use of the expression “and/or” between the last two members of a list of options for selection indicates that a selection of one or more of the listed options is appropriate and may be made.

Unless otherwise stated, when the outer bounds of a range with respect to a feature of an embodiment of the present technology are noted in the disclosure, it should be understood that in the embodiment, the possible values of the feature may include the noted outer bounds as well as values in between the noted outer bounds.

As used herein, unless otherwise stated, adjectives such as “substantially”, “approximately” and “about” that modify a condition or relationship characteristic of a feature or features of an embodiment of the present technology, are to be understood to mean that the condition or characteristic is defined to within tolerances that are acceptable for operation of the embodiment for an application for which it is intended, or within variations expected from the measurement being performed and/or from the measuring instrument being used. When the term “about” and “approximately” precedes a numerical value, it is intended to indicate +/−15%, or +/−10%, or even only +/−5%, and in some instances the precise value. Furthermore, unless otherwise stated, the terms (e.g., numbers) used in this disclosure, even without such adjectives, should be construed as having tolerances which may depart from the precise meaning of the relevant term but would enable the invention or the relevant portion thereof to operate and function as described, and as understood by a person skilled in the art.

While this disclosure has been described in terms of certain embodiments and generally associated methods, alterations and permutations of the embodiments and methods will be apparent to those skilled in the art. The present disclosure is to be understood as not limited by the specific embodiments described herein.

Certain marks referenced herein may be common law or registered trademarks of third parties. Use of these marks is by way of example and shall not be construed as descriptive or limit the scope of this disclosure to material associated only with such marks.

We claim:

1. A composition comprising nano-elements of at least one water-insoluble collagen-synthesis stimulating compound (CSSC) having a molecular weight of 0.6 kiloDalton (kDa) or more, the nano-elements being dispersed in a polar carrier;
wherein at least 50% of the total volume of the nano-elements have an average diameter (Dv50) of 200 nm or less; and
wherein the CSSC(s) or the nano-elements made therefrom have a viscosity of 10⁷ mPa·s or less, as measured at at least one temperature between 20° C. and 80° C., and at a shear rate of 10 sec⁻¹.

2. The composition as claimed in claim 1, wherein the at least one CSSC is plasticized by a non-volatile liquid.

3. The composition as claimed in claim 2, wherein the non-volatile liquid is selected from a group comprising monofunctional or polyfunctional aliphatic esters, fatty esters, cyclic organic esters, aromatic esters, terpenes, aromatic alcohols, aromatic ethers, aldehydes and combinations thereof; the non-volatile liquid being non-soluble in the polar carrier.

4. The composition as claimed in claim 1, wherein the at least one CSSC is characterized by at least one, at least two, or at least three of the following properties:
i. the CSSC and/or a plasticized CSSC is insoluble in the polar carrier;
ii. the CSSC and/or a plasticized CSSC is biodegradable and/or biocompatible;
iii. the CSSC has at least one of a first melting temperature (Tm), a first softening temperature (Ts) and a first glass transition temperature (Tg) of at most 300° C.;
iv. the CSSC, if plasticized, has at least one of a second Tm, a second Ts and a second Tg of at most 300° C., the second Tm, Ts or Tg being lower than a respective first Tm, Ts or Tg;
v. the CSSC and/or a plasticized CSSC has each respectively at least one of a first and/or second Tm of at least 0° C.;
vi. the CSSC and/or a plasticized CSSC has each respectively at least one of a first and/or a second Tm between 0° C. and 300° C.;
vii. the CSSC and/or a plasticized CSSC has each respectively at least one of a first and/or a second Tg and Ts of −75° C. or more;
viii. the CSSC and/or a plasticized CSSC has each respectively at least one of a first and/or second Tg and Ts between −75° C. and 300° C.;
ix. the CSSC and/or a plasticized CSSC has a viscosity of 5×10⁶ mPa·s or less, as measured at at least one temperature between 20° C. and 80° C., and at a shear rate of 10 sec⁻¹;
x. the CSSC has a molecular weight of 0.7 kDa or more;
xi. the CSSC has a molecular weight of 500 kDa or less; and
xii. the CSSC has a molecular weight between 0.6 kDa and 500 kDa.

5. The composition as claimed in claim 1, wherein the at least one CSSC is selected from:
(I) a polymer selected from a group of polymer families comprising aliphatic polyesters, polyhydroxy-alkanoates, poly(alkene dicarboxylates), polycarbonates, aliphatic-aromatic copolyesters, polysaccharides, lignins, isomers thereof, copolymers thereof and combinations thereof; and
(II) a natural polymerizable CSSC selected from resins, gums and gum-resins.

6. The composition as claimed in claim 1, wherein the polar carrier has a boiling temperature (Tb_{c}) between 45° C. and 350° C.

7. The composition as claimed in claim 1, further comprising at least one of:
i. at least one surfactant being an emulsifier or an hydrotrope disposed in the polar carrier;
ii. a polar-carrier-insoluble active agent being miscible with the at least one CSSC disposed in the nano-elements;
iii. a polar-carrier-soluble active agent disposed in the polar carrier; and
iv. a skin penetration enhancer disposed in the polar carrier.

8. The composition as claimed in claim 7, wherein the polar-carrier-insoluble active agent and/or the polar-carrier-soluble active agent are selected from:
a) a cosmetic agent selected from a group comprising: anti-aging, anti-acne, anti-oxidant, bleaching and tanning agents;
b) a pharmaceutical agent selected from a group comprising: analgesics, anesthetics, anti-addiction agents, anti-bacterials, anti-convulsants, anti-dementia agents, anti-depressants, anti-emetics, anti-fungals, anti-gout agents, anti-inflammatory agents, anti-migraine agents, anti-myasthenic agents, anti-mycobacterials, anti-neoplastics, anti-obesity agents, anti-parasitics, anti-parkinson agents, anti-psychotics, anti-spasticity agents, anti-virals, anxiolytics, bipolar agents, blood glucose regulators, cardiovascular agents, central nervous system agents, contraceptives, dental and oral agents, gastrointestinal agents, genetic/enzyme/protein disorder agents, genitourinary agents, hormonal agents, hormone suppressants, immunological agents, infertility agents, inflammatory bowel disease agents, metabolic bone disease agents, ophthalmic agents, otic agents, respiratory tract agents, sexual disorder agents, skeletal muscle relaxants, sleep disorder agents and dietary supplements; and
c) an agrochemical and/or industrial agent selected from a group comprising: acaricides, algicides, animal repellents, anthelmintics, anti-moth agents, avicides, bactericides, chemo-sterilants, fertilizers, fungicides, herbicides, insect pheromones, insect repellents, insecticides, molluscicides, nematicides, nitrification inhibitors, ovicides, plant growth activators, rodenticides, termicides and virucides.

9. The composition as claimed in claim 1, wherein:
i. each constituent of the nano-elements has a vapor pressure of 40 Pascal (Pa) or less, as measured at a temperature of about 20° C.; and/or
ii. the nano-elements contain less than 0.2 wt. % of a volatile organic compound (VOC) by weight of the nano-elements.

10. The composition as claimed in claim 1, wherein the composition is a topical composition adapted for application to an external surface to be treated therewith.

11. A method for preparing a composition comprising a collagen-synthesis stimulating compound (CSSC), the method comprising the steps of:
a) providing at least one CSSC, wherein the CSSC, or each CSSC:
i. is water-insoluble;
ii. has a molecular weight of at least 0.6 kDa;
iii. has at least one of a first melting temperature (Tm), a first softening temperature (Ts), and a first glass transition temperature (Tg) of 300° C. or less; and
iv. has a first viscosity;
b) mixing the CSSC(s) with a non-volatile liquid miscible therewith, the mixing being at a mixing temperature equal to or higher than at least one of the first Tm, first Ts, and first Tg of the CSSC(s), whereby a homogeneous plasticized CSSC(s) is formed, the plasticized CSSC(s) having:
A—a second Tm, a second Ts, or a second Tg lower than the respective first Tm, first Ts, or first Tg by at least 5° C.; and
B—a second viscosity lower than the first viscosity, at least the second viscosity being of 10⁷ mPa·s or less, as measured at at least one temperature between 20° C. and 80° C., and at a shear rate of 10 sec⁻¹;
c) combining a polar carrier with the plasticized CSSC(s); and
d) nano-sizing the combination of step c) by applying shear at a shearing temperature equal to or higher than at least one of the second Tm, second Ts, and second Tg of the plasticized CSSC(s), so as to obtain a nano-suspension, whereby nano-elements of plasticized CSSC are dispersed in the polar carrier;
wherein at least 50% of the total volume of the nano-elements have an average diameter (Dv50) of 200 nm or less.

12. The method as claimed in claim 11, wherein the at least one CSSC is further characterized by at least one, at least two, or at least three of the following properties:
i. the CSSC is insoluble in the polar carrier;
ii. the CSSC is biodegradable and/or biocompatible;
iii. the CSSC has at least one of a first Tm, Ts, or Tg of at most 250° C.;
iv. the CSSC has a first Tm of at least 0° C.;
v. the CSSC has a first Tm between 0° C. and 300° C.;
vi. the CSSC has at least one of a first Tg and a first Ts of −75° C. or more;
vii. the CSSC has at least one of a first Ts and a first Tg between −75° C. and 300° C.;
viii. the CSSC has a molecular weight of 0.7 kDa or more;
ix. the CSSC has a molecular weight of 500 kDa or less;
x. the CSSC has a molecular weight between 0.6 kDa and 500 kDa; and
xi. the CSSC has a first viscosity of 10⁷ mPa·s or less, as measured at at least one temperature between 20° C. and 80° C., and at a shear rate of 10 sec⁻¹.

13. The method as claimed in claim 11, wherein the homogeneous plasticized CSSC(s) has at least one of:
I) a second Tm within a range of from 0° C. to 290° C.; and
II) at least one of the second Ts and the second Tg within a range of from −75° C. to 290° C.

14. The method as claimed in claim 11, further comprising combining during the mixing of step b) at least one of:
i. a polar-carrier-insoluble non-ionic surfactant; and
ii. a polar-carrier-insoluble active agent.

15. The method as claimed in claim 11, further comprising during or after step c) or step d), dissolving within the polar carrier at least one of:
i. a polar-carrier-soluble surfactant;
ii. an intermediate emulsifier;
iii. a skin penetration enhancer; and
iv. a polar-carrier-soluble active agent.

16. The method as claimed in claim 11, wherein the polar carrier has a boiling temperature Tb_{c} at a pressure of nano-sizing and the non-volatile liquid has a boiling temperature Tbₗ at a pressure of mixing, the temperature of nano-sizing in the polar carrier being lower than Tb_{c} and the temperature of mixing with the non-volatile liquid being lower than Tbₗ.

17. The method as claimed in claim 11, wherein no volatile organic compound (VOC) is added to the CSSC so that the nano-elements contain less than 2 wt. % of a VOC by weight of the nano-elements.

18. A method for treating an animal subject, a plant or an object, the method comprising applying to a target surface on or surrounding the subject, the plant or the object a composition comprising nano-elements of a water-insoluble collagen-synthesis stimulating compound (CSSC) having a molecular weight of 0.6 kiloDalton (kDa) or more, the nano-elements being dispersed in a polar carrier;
wherein at least 50% of the total volume of the nano-elements have an average diameter (Dv50) of 200 nm or less; and
wherein the nano-elements have a viscosity of 10⁷ mPa·s or less, as measured at at least one temperature between 20° C. and 80° C., and at a shear rate of 10 sec⁻¹.

19. The method as claimed in claim 18, wherein the composition applied to the target surface is selected from:
i) a cosmetical composition for achieving a cosmetic effect improving skin appearance, wherein the target surface is a skin of a living subject, said effect including at least one of combating collagen, elastin or glycosaminoglycan breakdown, promoting collagen, elastin or glycosaminoglycan neo-synthesis, treating signs of ageing of the skin, combating wrinkles and fine lines, combating wizened skin, combating flaccid skin, combating thinned skin, combating dull, lifeless skin, combatting uneven skin pigmentation, combating lack of elasticity and/or tonicity of the skin, and skin bleaching or tanning;
ii) an agrochemical and/or an industrial composition for achieving an agrochemical effect, wherein the target surface is the surface of an inert object, a plant or a pest or the soil or the water in which plants are growing or pests are present, said effect being as one or more of an acaricide, an algicide, an anthelmintic, an anti-moth, an avicide, a bactericide, a chemo-sterilant, a fertilizer, a fungicide, a herbicide, an insecticide including an ovicide, an insect repellent, an insect pheromone, a molluscicide, a nematicide, a nitrification inhibitor, a pesticide, a pest repellent, a plant growth promotor, a rodenticide, a soil fertilizer, a termicide, a virucide, and a plant wound protectant; and
iii) a pharmaceutical composition for achieving a pharmaceutic effect, wherein the target surface is a skin of a living subject, said effect being selected from:
A—improving skin health and/or appearance, the effect including at least one of treating skin lesions, restoring skin integrity, promoting wounds healing, alleviating or treating local inflammation, skin irritation, skin infection, skin rashes, skin pain, skin burns, skin swelling and/or skin allergy; and promoting or inhibiting hair growth; and/or
B—delaying, alleviating, preventing or treating a transdermally treatable disorder, the pharmaceutical composition comprising active agents selected from: analgesics, anesthetics, anti-addiction agents, anti-bacterials, anti-convulsants, anti-dementia agents, anti-depressants, anti-emetics, anti-fungals, anti-gout agents, anti-inflammatory agents, anti-migraine agents, anti-myasthenic agents, anti-mycobacterials, anti-neoplastics, anti-obesity agents, anti-parasitics, anti-parkinson agents, anti-psychotics, anti-spasticity agents, anti-virals, anxiolytics, bipolar agents, blood glucose regulators, cardiovascular agents, central nervous system agents, contraceptives, dental and oral agents, gastrointestinal agents, genetic/enzyme/protein disorder agents, genitourinary agents, hormonal agents, hormone suppressants, immunological agents, infertility agents, inflammatory bowel disease agents, metabolic bone disease agents, ophthalmic agents, otic agents, respiratory tract agents, sexual disorder agents, skeletal muscle relaxants, sleep disorder agents and dietary supplements.

20. The method as claimed in claim 18, wherein:
A) the target surface is a skin of a human or non-human animal subject and the composition is a dermatological composition in a form selected from a group comprising creams, foams, gels, lotions and ointments, the application being by depositing the dermatological composition on the skin, or by attaching to the skin a dressing including the dermatological composition; or
B) the target surface is the soil or the water in which plants are growing or pests are present and the surfaces of the plants and the pest, and the composition is an agrochemical composition in a form selected from a group comprising solutions, emulsions, powders, granules, pellets and aerosols, the application being by spreading, dispersing or spraying the agrochemical composition on the target surface; or
C) the target surface is of an inert object and the composition is an industrial composition in a form selected from a group comprising paints, pastes, varnishes, lacquers, sprays and powders, the application being by spreading, spraying or dispersing the industrial composition on the inert surface.
